(19) United States
(12) Patent Application Publication
(10) Pub. No.: US 2007/0027118 A1
(43) Pub. Date: Feb. 1, 2007

(54) Novel compounds of amino sulfonyl derivatives

(75) Inventors: Hengmiao Cheng, San Diego, CA (US); Stephan James Cripps, San Diego, CA (US); Klaus Ruprecht Dress, San Diego, CA (US); Buwen Huang, San Diego, CA (US); Sajiv Krishnan Nair, Vista, CA (US); Yong Wang, San Diego, CA (US)

Correspondence Address: PFIZER INC, 10555 SCIENCE CENTER DRIVE, SAN DIEGO, CA, 92121, US

(73) Assignee: AGOURON PHARMACEUTICALS, INC

(21) Appl. No.: 11/398,810

(22) Filed: Apr. 6, 2006

Related U.S. Application Data
(60) Provisional application No. 60/669,613, filed on Apr. 7, 2005.
(60) Provisional application No. 60/772,495, filed on Feb. 10, 2006.

(51) Int. Cl. A61K 31/655; A61K 31/44; A61K 31/4439; C07D 401/02; C07D 213/72

(52) U.S. Cl. 514/151; 514/352; 514/340; 546/268.1; 546/304

(57) ABSTRACT

The present invention relates to compounds with formula (I) or a pharmaceutically acceptable salt thereof: 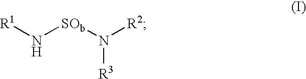 wherein;

T is a (4 to 10)-membered heterocyclyl and wherein R¹, R² and R³ are as defined in the specification. The invention also relates to pharmaceutical compositions comprising the compounds of formula (I) and methods of treating a condition that is mediated by the modulation of the 11-β-hsd-1 enzyme.

This application claims the benefit of U.S. Provisional Application No. 60/669,613 filed Apr. 7, 2005, and U.S. Provisional Application No. 60/772,495 filed Feb. 10, 2006, the contents of which are hereby incorporated by reference in their entireties.

### FIELD OF THE INVENTION

The present invention relates to novel compounds, to pharmaceutical compositions comprising the compounds, as well as to the use of the compounds in medicine and for the preparation of a medicament which acts on the human 11-β-hydroxysteroid dehydrogenase type 1 enzyme (11-β-hsd-1).

### BACKGROUND OF THE INVENTION

It has been known for more than half a century that glucocorticoids have a central role in diabetes. For example, the removal of the pituitary or the adrenal gland from a diabetic animal alleviates the most severe symptoms of diabetes and lowers the concentration of glucose in the blood (Long, C. D. and F. D. W. Leukins (1936) J. Exp. Med. 63: 465-490; Houssay, B. A. (1942) Endocrinology 30: 884-892). Additionally, it is also well established that glucocorticoids enable the effect of glucagon on the liver.

The role of 11-β-hsd-1 as an important regulator of local glucocorticoid effects and thus of hepatic glucose production is well substantiated (see e.g. Jamieson et al. (2000) J. Endocrinol. 165: p. 685-692). The hepatic insulin sensitivity was improved in healthy human volunteers treated with the non-specific 11-β-hsd-1 inhibitor carbenoxolone (Walker, B. R., et al. (1995) J. Clin. Endocrinol. Metab. 80: 3155-3159). Furthermore, the expected mechanism has been established by different experiments with mice and rats. These studies showed that the mRNA levels and activities of two key enzymes in hepatic glucose production were reduced, namely the rate-limiting enzyme in gluconeogenesis, phosphoenolpyruvate carboxykinase (PEPCK), and glucose-6-phosphatase (G6Pase) catalyzing the last common step of gluconeogenesis and glycogenolysis. Finally, the blood glucose level and hepatic glucose production was reduced in mice having the 11-β-hsd-1 gene knocked-out. Data from this model also confirms that inhibition of 11-β-hsd-1 will not cause hypoglycemia, as predicted, since the basal levels of PEPCK and G6Pase are regulated independently of glucocorticoids (Kotelevtsev, Y., et al., (1997) Proc. Natl. Acad. Sci. USA 94:14924-14929).

Abdominal obesity is closely associated with glucose intolerance, hyperinsulinemia, hypertriglyceridemia, and other factors of the so-called Metabolic Syndrome (e.g. raised blood pressure, decreased levels of HDL and increased levels of VLDL) (Montague & O'Rahilly, Diabetes 49: 883-888, 2000). Obesity is an important factor in Metabolic Syndrome as well as in the majority (>80%) of type 2 diabetic, and omental fat appears to be of central importance. Inhibition of the enzyme in pre-adipocytes (stromal cells) has been shown to decrease the rate of differentiation into adipocytes. This is predicted to result in diminished expansion (possibly reduction) of the omental fat depot, i.e. reduced central obesity (Bujalska, I. J., Kumar, S., and Stewart, P. M. (1997) Lancet 349: 1210-1213).

The compounds of the present invention are 11 β-hsd-1 inhibitors, and are therefore believed to be useful in the treatment of diabetes, obesity, glaucoma, osteoporosis, cognitive disorders, immune disorders, depression, hypertension, and metabolic diseases.

### SUMMARY OF THE INVENTION

An embodiment of the invention relates to a compound of formula (I): 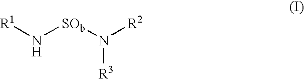
wherein:

R¹ is 2-pyridinyl which is fused or substituted with 1-3 R⁶ groups, with at least one R⁶ group being at the 6′ position of the pyridinyl;

b is 2;

R² and R³ are taken together with the nitrogen atom to which they are attached to form a (4 to 11)-membered heterocyclyl, and the (4 to 11)-membered heterocyclyl may optionally be substituted by 1 to 3 R⁶ groups;

the carbon atoms of R¹, R², and R³ may each be optionally substituted by 1 to 3 R⁶ groups;

each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³ R¹⁴, —NR¹³(C═O)—R¹⁴, —(C═O)—NR¹³R¹⁴, —R¹³—(C═)—NR¹⁴R¹⁵, —NR¹³R¹⁴, —NR¹³OR¹⁴, —S(O)NR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)ᵥ(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl;

k is selected from 1 and 2;

j is selected from the group consisting of 0, 1, and 2;

t, u, p, q, and v are each independently selected from the group consisting of 0, 1, 2, 3, 4, and 5;

any 1 or 2 carbon atoms of any foregoing (4 to 11)-membered heterocyclyl groups may be optionally substituted with an oxo (═O);

any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(CO)—NR²¹R²², —NR²¹R²², —NR²¹OR²², (C₁-C₆)alkyl, (C₂-C₆)alkenyl, (C₂-C₆)alkynyl, —(CR²¹R²²)ᵤ(C₆-C₁₂)aryl, and —(CR²¹R²²)ᵤ(4 to 11)-membered heterocyclyl;

each R⁷, R⁸, R⁹, R¹⁰, R¹¹, R¹², R¹³, R¹⁴, R¹⁵, R¹⁶ , R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)ₚ(4 to 11)-membered heterocyclyl;

any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);

each R²³ and R²⁴ is independently selected from H and (C₁-C₆)alkyl;

and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;

with the proviso that —NR²R³ is not an unsubstituted group selected from 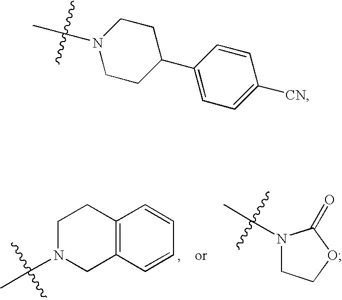
and the further proviso that when —R¹ is 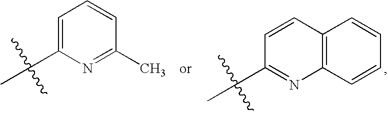
then —NR²R³ is not an unsubstituted or substituted, fused or unfused group selected from

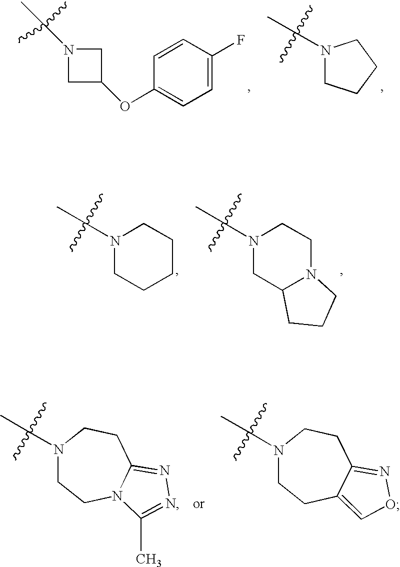

or a pharmaceutically acceptable salt or solvate thereof.

In one embodiment, the invention relates to a compound of formula (I), wherein R¹ is 2-pyridinyl substituted with 1 to 3 R⁶ groups wherein at least one R⁶ group is at the 6′ position of the pyridinyl. In a further embodiment, R¹ is quinolinyl.

In yet another embodiment, the invention relates to a compound of formula (I) wherein R² and R³ are taken together to form a 6-membered heterocyclyl containing at least one nitrogen atom. In a further embodiment, the 6-membered heterocyclyl is piperazinyl. In still another embodiment, the 6-membered heterocycle is piperidinyl.

In still another embodiment, the invention relates to a compound of formula (I) wherein R² and R³ are taken together to form a 10-membered heterocyclyl containing at least one nitrogen atom.

In yet another embodiment, the invention relates to a compound of formula (I), wherein R² and R³ are taken together to form an 11-membered heterocyclyl containing at least one nitrogen atom. In a further embodiment, the 11-membered heterocyclyl is benzazepinyl.

In another embodiment, the invention relates to a compound selected from the group consisting of:

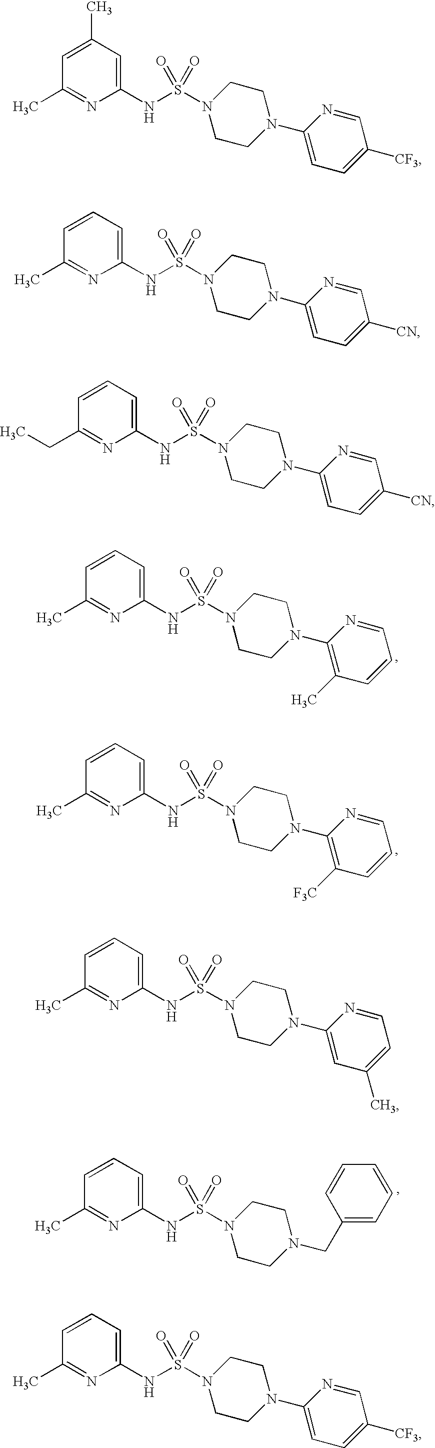

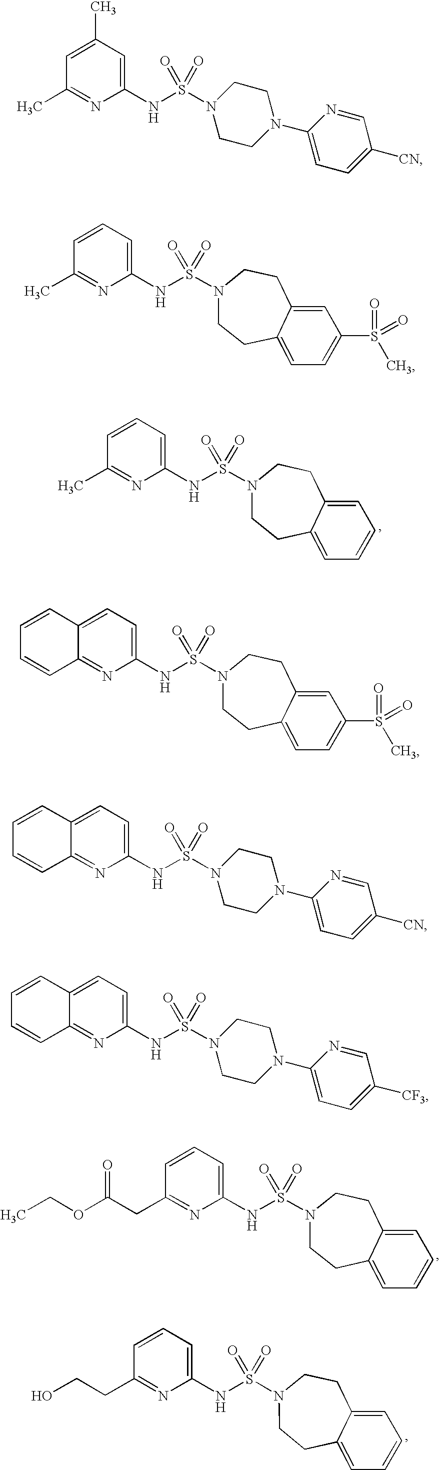

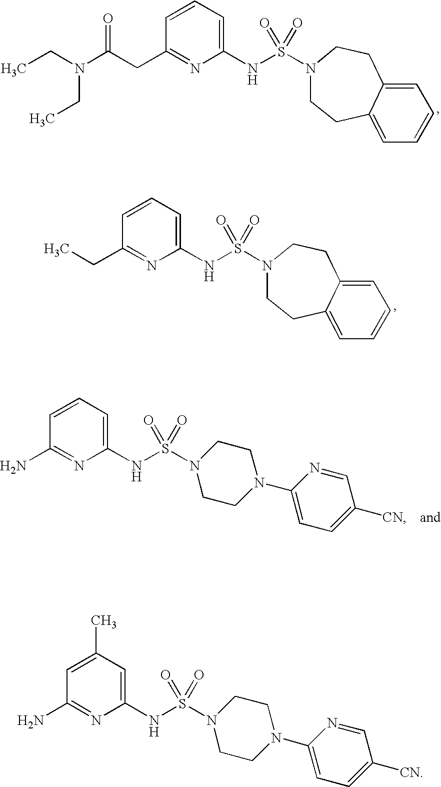

or a pharmaceutically acceptable salt or solvate thereof.

In still another embodiment, the invention relates to a compound selected from the group consisting of

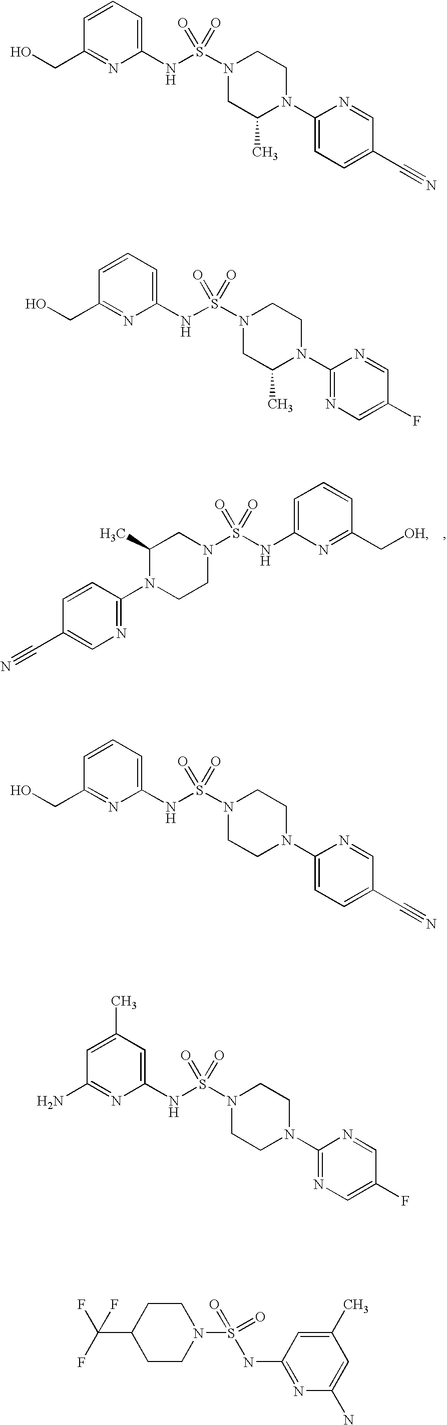

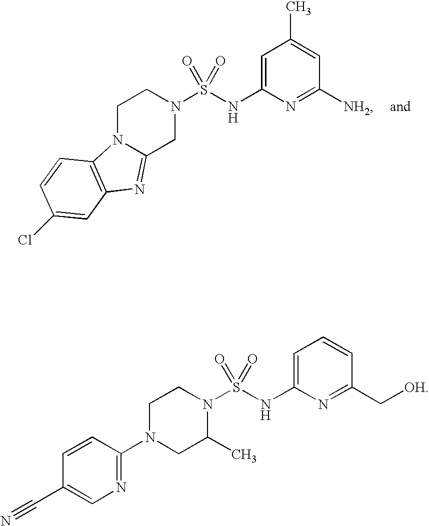

or a pharmaceutically acceptable salt or solvate thereof.

In still another embodiment, the invention relates to a compound of formula (III): 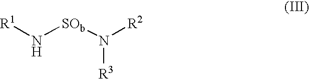
wherein:

R¹ is pyridinyl which is fused or unfused, unsubstituted or substituted with 1-3 R⁶ groups; —(CR⁴R⁵)ₜ(C₆-C₁₂)aryl, and —(CR⁴R⁵)ₜ(4 to 10)-membered heterocyclyl;

b is 2;

R² and R³ are taken together with the nitrogen atom to which they are attached to form a (12-14)-membered heterocyclyl, and the (12-15)-membered heterocyclyl may optionally be substituted by 1 to 3 R⁶ groups;

each R⁴ and R⁵ is independently selected from H and (C₁-C₆)alkyl; the carbon atoms of R¹, R², R³, R⁴, and R⁵ may each be optionally substituted by 1 to 3 R⁶ groups;

each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)(C₆-C₁₂)aryl —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl;

k is selected from 1 and 2;

j is selected from the group consisting of 0, 1, and 2;

t, u, p, q, and v are each independently selected from the group consisting of 0, 1, 2, 3, 4, and 5;

any 1 or 2 carbon atoms of any foregoing (4 to 11)-membered heterocyclyl group may be optionally substituted with an oxo (═O);

any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₆)alkenyl, (C₂-C₆)alkynyl, —(CR²¹R²²)ᵤ(C₆-C₁₂)aryl, and —(CR²¹R²²)ᵤ(4 to 11)-membered heterocyclyl;
each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)ₚ(4 to 11)-membered heterocyclyl;

any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);

each R²³ and R²⁴ is independently selected from H and (C₁-C₆)alkyl;

and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;

or a pharmaceutically acceptable salt or solvate thereof.

In a further embodiment, the invention relates to a compound of formula (III) wherein —NR²R³ is a 13-membered heterocyclic optionally substituted with 1 to 3 R⁶ groups. In a further embodiment of this invention, —NR²R³ is a fused tricyclic group such as 3,4-dihydropyrazino[1,2-a]benzimidazole. In an alternate embodiment of this invention, —NR²R³ is a spirocyclic group such as 3,4-dihydro-1′H-spirochromene.

In another embodiment, the invention relates to a pharmaceutical composition comprising an effective amount of a compound according to formula (I) or (III), or a pharmaceutically acceptable salt or solvate thereof, and a pharmaceutically acceptable carrier.

In yet another embodiment, the invention relates to a method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to formula (I) or (III), or a pharmaceutically acceptable salt or solvate thereof.

An embodiment of the invention relates to a method of preparing a compound of formula (I) 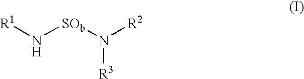
wherein:

R¹ is a —(CR⁴R)ₜ(4 to 10)-membered heterocyclyl;

b and k are each independently selected from 1 and 2;

j is selected from the group consisting of 0, 1, and 2;

t, u, p, q, and v are each independently selected from the group consisting of 0, 1, 2, 3, 4, and 5;

each R² and R³ is independently selected from the group consisting of H, (C₁-C₆) alkyl, (C₂-C₆) alkenyl, (C₂-C₆) alkynyl, —(CR⁴R)ₜ(C₃-C₁₀)cycloalkyl, —(CR⁴R⁵)ₜ(C₆-C₁₀)aryl, and —(CR⁴R⁵)ₜ(4 to 11)-membered heterocyclyl;

or R² and R³ may optionally be taken together with the nitrogen atom to which they are attached to form a (4 to 11)-membered heterocyclyl, and the (4 to 11)-membered heterocyclyl may be optionally substituted by 1 to 3 R⁶ groups;

each R⁴ and R⁵ is independently selected from H and (C₁-C₆)alkyl; the carbon atoms of R¹, R², R³, R⁴, and R⁵ may each be optionally substituted by 1 to 3 R⁶ groups;

each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl;

any 1 or 2 carbon atoms of any foregoing (4 to 11)-membered heterocyclyl group may be optionally substituted with an oxo (═O);

any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₆)alkenyl, (C₂-C₆)alkynyl, —(CR²¹R²² )ᵤ(C₆-C₁₂)aryl, and —(CR²¹R²²)ᵤ(4 to 11)-membered heterocyclyl;

each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)(C₆-C₁₂)aryl, and —(CR²³R²⁴)ₚ(4 to 11)-membered heterocyclyl;

any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);

each R²³ and R²⁴ is independently selected from H and (C₁-C₆)alkyl; comprising the steps of:

(a) treating a compound of formula (II), 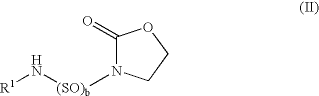
wherein:

R¹ and b are defined as above;

with R²R³NH in the presence of a base in a solvent, wherein each R² and R³ is defined as above.

In an embodiment of the invention, the invention relates to the method according to formula (II), wherein the base in step (a) is triethylamine or diisopropylethylamine.

In another embodiment, the invention relates to the method according to formula (II), wherein the solvent in step (a) is selected from the group consisting of acetonitrile, DMF, and a mixture of acetonitrile and DMF.

In yet another embodiment, the invention relates to the method according formula (II), wherein step (a) proceeds at a temperature range from about 70° C. to about 100° C.

In yet another embodiment, the invention relates to the method according to formula (II), wherein step (a) proceeds overnight.

In yet another embodiment, the invention relates to the method according to formula (II), wherein step (a) proceeds at a temperature range from about 70° C. to about 140° C.

In yet another embodiment, the invention relates to the method according to formula (II), wherein step (a) proceeds at a time from about 10 minutes to about 2 hours in a microwave.

An embodiment of the invention relates to a method of preparing a compound of formula (II),

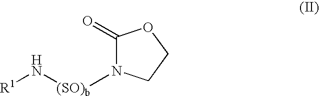

wherein:

k and b are each independently selected from 1 and 2;

j is selected from the group consisting of 0, 1, and 2;

t, u, p, q, and v are each independently selected from the group consisting of 0, 1, 2, 3, 4, and 5;

R¹ is a —(CR⁴R⁵)ₜ(4 to 10)-membered heterocyclyl, and may be optionally substituted by 1 to 3 R⁶ groups;

each R⁴ and R⁵ is independently selected from H and (C₁-C₆)alkyl;

each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)_{q}(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)ᵥ(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl,

any 1 or 2 carbon atoms of any foregoing (4 to 11)-membered heterocyclyl group may be optionally substituted with an oxo (═O);

any (C₁-C₆)alkyl, any (C₆-C₁₂)aryl, and any (4 to 11)-membered heterocyclyl of the foregoing R⁶ groups may be optionally substituted with 1 to 3 substituents independently selected

from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₆)alkenyl, (C₂-C₆)alkynyl, —(CR²¹R²²)_{u(C}₆-C₁₂)aryl, and —(CR²¹R²²)ᵤ(4 to 11)-membered heterocyclyl;

each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)ₚ(4 to 11)-membered heterocyclyl;

any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);

each R²³ and R²⁴ is independently selected from H and (C₁-C₆)alkyl;

comprising the steps of:

(b) reacting chlorosulfonyl isocyanate and 2-chloroethanol; and

(c) adding an amine of formula NR¹H₂, wherein R¹ is a —(CR⁴R⁵)ₜ(4 to 10)-membered heterocyclyl with a base in a solvent.

In another embodiment, the invention relates to the method according to formula (II), wherein step (b) proceeds at a temperature from about 0° C. to about 5° C.

In yet another embodiment, the invention relates to the method according to formula (II), wherein step (b) proceeds at a time for about 0.25 hours to about 2 hours.

In yet another embodiment, the invention relates to the method according to formula (II), wherein the base in step (c) is triethylamine or diisopropylethylamine.

In yet another embodiment, the invention relates to the method according to formula (II), wherein the solvent in step (c) is dichloromethane.

In an embodiment of the invention, the invention relates to the method according to formula (II), wherein step (c) proceeds at a temperature range from about 0° C. to about 5° C.

In another embodiment, the invention relates to the method according to formula (II), wherein step (c) further proceeds at a temperature of about 0° C. for about 2 hours.

In yet another embodiment, the invention relates to the method according to formula (II), wherein step (c) further proceeds at a temperature of about 25° C. at a range of time from about 0 hours to 24 hours.

An embodiment of the invention relates to a compound of formula 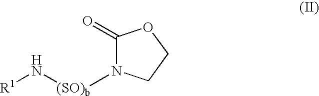
wherein:

k and b are each independently selected from 1 and 2;

j is selected from the group consisting of 0, 1, and 2;

t, u, p, q, and v are each independently selected from the group consisting of 0, 1, 2, 3, 4, and 5;

R¹ is —(CR⁴R)ₜ(4 to 10)-membered heterocyclyl and may be optionally substituted by 1 to 3 R⁶ groups;

each R⁴ and R⁵ is independently selected from H and (C₁-C₆)alkyl;

each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)ᵥ(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl;

any 1 or 2 carbon atoms of any foregoing (4 to 11)-membered heterocyclyl group may be optionally substituted with an oxo (═O);

any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₆)alkenyl, (C₂-C₆)alkynyl, —(CR²¹R²²)ᵤ(C₆-C₁₂)aryl, and —(CR²¹R²²)ᵤ(4 to 11)-membered heterocyclyl;

each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)(4 to 11)-membered heterocyclyl;

any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);

each R²³ and R²⁴ is independently selected from H and (C₁-C₆)alkyl.

### DEFINITIONS

As used herein, the terms “comprising” and “including” are used in their open, non-limiting sense.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least-one carbon-carbon triple bond wherein alkyl is as defined above.

The term “alkoxy”, as used herein, unless otherwise indicated, includes O-alkyl groups wherein alkyl is as defined above.

The term “amino”, as used herein, unless otherwise indicated, is intended to include the —NH₂ radical, and any substitutions of the N atom.

The terms “halogen” and “halo”, as used herein, unless otherwise indicated, represent chlorine, fluorine, bromine or iodine.

The term “trifluoromethyl”, as used herein, unless otherwise indicated, is meant to represent a —CF₃ group.

The term “trifluoromethoxy”, as used herein, unless otherwise indicated, is meant to represent a —OCF₃ group.

The term “cyano”, as used herein, unless otherwise indicated, is meant to represent a —CN group.

The term “CH₂Cl₂”, as used herein, unless otherwise indicated, is meant to represent dichloromethane.

The term, “OMs”, as used herein, unless otherwise indicated, is intended to mean methanesulfonate.

The term “Me”, as used herein, unless otherwise indicated, is intended to mean methyl.

The term “MeOH” as used herein, unless otherwise indicated, is intended to mean methanol.

The term “Et”, as used herein, unless otherwise indicated, is intended to mean ethyl.

The term “Et₂O”, as used herein, unless otherwise indicated, is intended to mean diethylether.

The term “EtOH”, as used herein, unless otherwise indicated, is intended to mean ethanol.

The term “Et₃N”, as used herein, unless otherwise indicated, is intended to mean triethylamine.

The term “EtOAc”, as used herein, unless otherwise indicated, is ethyl acetate.

The term “AlMe₂Cl” as used herein, unless otherwise indicated, is intended to mean dimethyl aluminum chloride.

The term “Ac”, as used herein, unless otherwise indicated, is intended to mean acetyl.

The term “DAST”, as used herein, unless otherwise indicated, is intended to mean diethylamino sulfur trifluoride.

The term “TFA”, as used herein, unless otherwise indicated, is intended to mean trifluoroacetic acid.

The term “TEA”, as used herein, unless otherwise indicated, is intended to mean triethanolamine.

The term “LAH”, as used herein, unless otherwise indicated, is intended to mean lithium aluminum hydride.

The term “HATU”, as used herein, unless otherwise indicated, is intended to mean N,N,N′,N′-tetramethyluronium hexafluorophosphate.

The term “THF”, as used herein, unless otherwise indicated, is intended to mean tetrahydrofuran.

The term “TIOH”, as used herein, unless otherwise indicated, is intended to mean thallium(I) hydroxide.

The term “TIOEt”, as used herein, unless otherwise indicated, is intended to mean thallium(I) ethoxide.

The term “PCy₃”, as used herein, unless otherwise indicated, is intended to mean tricyclohexylphosphine.

The term “Pd₂(dba)₃”, as used herein, unless otherwise indicated, is intended to mean tris(dibenzylideneacetone)dipalladium(0).

The term “Pd(OAc)₂”, as used herein, unless otherwise indicated, is intended to mean palladium(II) acetate.

The term “Pd(PPh₃)₂Cl₂”, as used herein, unless otherwise indicated, is intended to mean dichlorobis(triphenylphosphine)palladium(II).

The term “Pd(PPh₃)₄”, as used herein, unless otherwise indicated, is intended to mean tetrakis(triphenylphophine)palladium(0).

The term “Pd(dppf)Cl₂”as used herein, unless otherwise indicated, is intended to mean (1,1′-bis(diphenylphosphino)ferrocene)dichloropalladium(II), complex with dichloromethane (1:1).

The term “G6P”, as used herein, unless otherwise indicated, is intended to mean glucose-6-phosphate.

The term “NIDDM”, as used herein, unless otherwise indicated, is intended to mean non insulin dependent diabetes mellitus

The term “NADPH”, as used herein, unless otherwise indicated, is intended to mean nicotinamide adenine dinucleotide phosphate, reduced form.

The term “CDCl₃” or CHLORFORM-D”, as used herein, unless otherwise indicated, is intended to mean deuterochloroform.

The term “CD₃OD”, as used herein, unless otherwise indicated, is intended to mean deuteromethanol.

The term “CD₃CN”, as used herein, unless otherwise indicated, is intended to mean deuteroacetonitrile.

The term “DEAD”, as used herein, unless otherwise indicated, is intended to mean diethyl azodicarboxylate.

The term “TsCH₂NC”, as used herein, unless otherwise indicated, is intended to mean tosylmethyl isocyanide.

The term “ClSO₃H”, as used herein, unless otherwise indicated, is intended to mean chlorosulfonic acid.

The term “DMSO-d₆ or DMSO-D₆”, as used herein, unless otherwise indicated, is intended to mean deuterodimethyl sulfoxide.

The term “DME”, as used herein, unless otherwise indicated, is intended to mean 1,2-dimethoxyethane.

The term “DMF”, as used herein, unless otherwise indicated, is intended to mean N,N-dimethylformamide.

The term “DMSO”, as used herein, unless otherwise indicated, is intended to mean, unless otherwise indicated dimethylsulfoxide.

The term “DIEA”, as used herein, unless otherwise indicated, is intended to mean diisopropylethylamine.

The term “prep-TLC”, as used herein, unless otherwise indicated, is intended to mean preparative thin layer chromatography.

The term “DI”, as used herein, unless otherwise indicated, is intended to mean deionized.

The term “KOAc”, as used herein, unless otherwise indicated, is intended to mean potassium acetate.

The term “neat”, as used herein, unless otherwise indicated, is meant to represent an absence of solvent.

The term “mmol”, as used herein, unless otherwise indicated, is intended to mean millimole.

The term “equiv”, as used herein, unless otherwise indicated, is intended to mean equivalent.

The term “mL”, as used herein, unless otherwise indicated, is intended to mean milliliter.

The term “U”, as used herein, unless otherwise indicated, is intended to mean units.

The term “mm” as used herein, unless otherwise indicated, is intended to mean millimeter.

The term “g”, as used herein, unless otherwise indicated, is intended to mean gram.

The term “kg”, as used herein, unless otherwise indicated, is intended to mean kilogram.

The term “h”, as used herein, unless otherwise indicated, is intended to mean hour.

The term “min”, as used herein, unless otherwise indicated, is intended to mean minute.

The term “μL”, as used herein, unless otherwise indicated, is intended to mean microliter.

The term “μM”, as used herein, unless otherwise indicated, is intended to mean micromolar.

The term “μm”, as used herein, unless otherwise indicated, is intended to mean micrometer.

The term “M”, as used herein, unless otherwise indicated, is intended to mean molar.

The term “N”, as used herein, unless otherwise indicated, is intended to mean normal.

The term “nm”, as used herein, unless otherwise indicated, is intended to mean nanometer.

The term “nM”, as used herein, unless otherwise indicated, is intended to mean nanoMolar.

The term “amu”, as used herein, unless otherwise indicated, is intended to mean atomic mass unit.

The term “° C.”, as used herein, unless otherwise indicated, is intended to mean Celsius.

The term “m/z”, as used herein, unless otherwise indicated, is intended to mean, mass/charge ratio.

The term “wt/wt, as used herein, unless otherwise indicated, is intended to mean weight/weight.

The term “v/v”, as used herein, unless otherwise indicated, is intended to mean volume/volume.

The term “mL/min”, as used herein, unless otherwise indicated, is intended to mean milliliter/minute.

The term “UV”, as used herein, unless otherwise indicated, is intended to mean ultraviolet.

The term “APCI-MS”, as used herein, unless otherwise indicated, is intended to mean atmospheric pressure chemical ionization mass spectroscopy.

The term “HPLC”, as used herein, unless otherwise indicated, is intended to mean high performance liquid chromatograph. The chromatography was performed at a temperature of about 20° C., unless otherwise indicated.

The term “LC”, as used herein, unless otherwise indicated, is intended to mean liquid chromatograph.

The term “LCMS”, as used herein, unless otherwise indicated, is intended to mean liquid chromatography mass spectroscopy.

The term “TLC”, as used herein, unless otherwise indicated, is intended to mean thin layer chromatography.

The term “SFC”, as used herein, unless otherwise indicated, is intended to mean supercritical fluid chromatography.

The term “sat” as used herein, unless otherwise indicated, is intended to mean saturated.

The term “aq” as used herein, is intended to mean aqueous.

The term “ELSD” as used herein, unless otherwise indicated, is intended to mean evaporative light scattering detection.

The term “MS”, as used herein, unless otherwise indicated, is intended to mean mass spectroscopy.

The term “HRMS (ESI)”, as used herein, unless otherwise indicated, is intended to mean high-resolution mass spectrometry (electrospray ionization).

The term “Anal.”, as used herein, unless otherwise indicated, is intended to mean analytical.

The term “Calcd”, as used herein, unless otherwise indicated, is intended to mean calculated.

The term “N/A”, as used herein, unless otherwise indicated, is intended to mean not tested.

The term “RT”, as used herein, unless otherwise indicated, is intended to mean room temperature.

The term “Mth.”, as used herein, unless otherwise indicated, is intended to mean Method.

The term “Celite®”, as used herein, unless otherwise indicated, is intended to mean a white solid diatomite filter agent commercially available from World Minerals located in Los Angeles, Calif. USA.

The term “Eg.”, as used herein, unless otherwise indicated, is intended to mean example.

Terms such as —(CR³R⁴)ₜ or —(CR¹⁰R¹¹)ᵥ, for example, are used, R³, R⁴, R¹⁰ and R¹¹ may vary with each iteration of t or v above 1. For instance, where t or v is 2 the terms —(CR³R⁴)ᵥ or —(CR¹⁰R¹¹)ₜ may equal —CH₂CH₂—, or —CH(CH₃)C(CH₂CH₃)(CH₂CH₂CH₃)—, or any number of similar moieties falling within the scope of the definitions of R³, R⁴, R¹⁰ and R¹¹.

The term “Kᵢ”, as used herein, unless otherwise indicated, is intended to mean values of enzyme inhibition constant.

The term “Kᵢ app”, as used herein, unless otherwise indicated, is intended to mean Kᵢ apparent The term “IC₅₀”, as used herein, unless otherwise indicated, is intended to mean concentrations required for at least 50% enzyme inhibition.

The term “substituted” means that the specified group or moiety bears one or more substituents.

The term “unsubstituted” means that the specified group bears no substituents.

The term “optionally substituted” means that the specified group is unsubstituted or substituted by one or more substituents.

In accordance with convention, in some structural formula herein, the carbon atoms and their bound hydrogen atoms are not explicitly depicted e.g., [symbol] represents a methyl group, 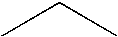
represents an ethyl group, 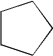
represents a cyclopentyl group, etc.

The term “cycloalkyl”, as used herein, unless otherwise indicated, refers to a non-aromatic, saturated or partially saturated, monocyclic or fused, spiro or unfused bicyclic or tricyclic hydrocarbon referred to herein containing a total of from 3 to 10 carbon atoms, suitably 5-8 ring carbon atoms. Exemplary cycloalkyls include rings having from 3-10 carbon atoms, such as cyclopropyl, cyclobutyl, cyclopentyl, cyclohexyl, cycloheptyl, and adamantyl. Illustrative examples of cycloalkyl are derived from, but not limited to, the following:

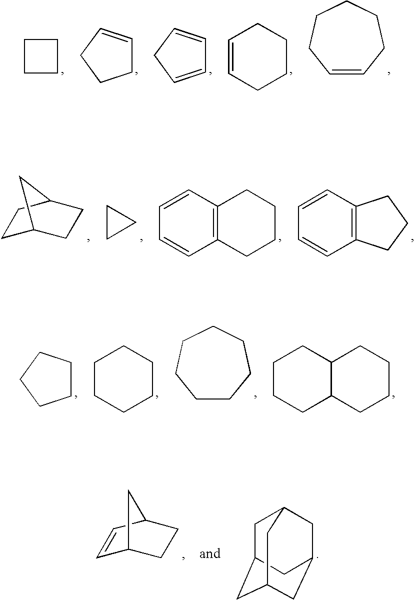

The term “aryl”, as used herein, unless otherwise indicated, includes an organic radical derived from an aromatic hydrocarbon by removal of one hydrogen, such as phenyl or naphthyl.

The term “(3-7)-membered heterocyclyl”, “(6-10)-membered heterocyclyl”, or “(4 to 10)-membered heterocyclyl”, as used herein, unless otherwise indicated, includes aromatic and non-aromatic heterocyclic groups containing one to four heteroatoms each selected from O, S and N, wherein each heterocyclic group has from 3-7, 6-10, or 4 to 10 atoms, respectively, in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3 membered heterocyclic group is aziridine, an example of a 4 membered heterocyclic group is azetidinyl (derived from azetidine). An example of a 5 membered heterocyclic group is thiazolyl, an example of a 7 membered ring is azepinyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4 to 10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4 to 10 membered heterocyclic are derived from, but not limited to, the following: 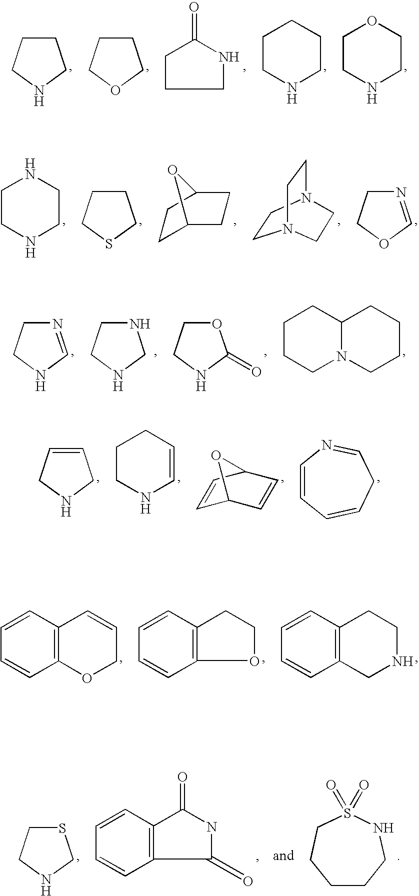
The term “(12-15)-membered heterocyclyl”, as used herein, unless otherwise indicated, includes aromatic and non-aromatic heterocyclic groups that are in a partially fused or spirocyclic configuration and which contain at least one N and optionally additional 1 to 5 heteroatoms each selected from O, S and N, wherein the heterocyclic group has from 12 to 15 atoms, respectively, in its system, and with the proviso that any ring of said group does not contain two adjacent O or S atoms. Non-aromatic rings of the heterocyclic group include groups having only 3 atoms in their ring system, but aromatic heterocyclic groups must have at least 5 atoms in their ring system. The heterocyclic groups include tricyclic fused ring and spirocyclic systems. An example of a 13-membered tricyclic heterocyclic group is 3,4-dihydropyrazino[1,2-a]benzimidazole and an example of a 15-membered spirocyclic heterocyclic group is 3,4-dihydro-1′H-spirochromene.

Unless otherwise indicated, the term “oxo” refers to ═O.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO (dimethylsulfoxide), ethyl acetate, acetic acid, or ethanolamine.

The phrase “pharmaceutically acceptable salt(s)”, as used herein, unless otherwise indicated, includes salts of acidic or basic groups which may be present in the compounds of formula (I) or formula (II). The compounds of formula (I) or formula (II)that are basic in nature are capable of forming a wide variety of salts with various inorganic and organic acids. The acids that may be used to prepare pharmaceutically acceptable acid addition salts of such basic compounds of formula (I) or formula (II) are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The term “diseases in which the liver is a target organ”, as used herein, unless otherwise indicated means diabetes, hepatitis, liver cancer, liver fibrosis, and malaria.

The term “Metabolic syndrome”, as used herein, unless otherwise indicated means psoriasis, diabetes mellitus, wound healing, inflammation, neurodegenerative diseases, galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m², and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels”, as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being preferred. As noted previously, since the activity of different compounds which fall within the definition of formula (I) or formula (II) as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, NIDDM, arterial hypertension, central (visceral) obesity, and dyslipidemia.

Certain compounds of formula (I) or formula (II) may have asymmetric centers and therefore exist in different enantiomeric forms. All optical isomers and stereoisomers of the compounds of formula (I) or formula (II), and mixtures thereof, are considered to be within the scope of the invention. With respect to the compounds of formula (I) or formula (II), the invention includes the use of a racemate, one or more enantiomeric forms, one or more diastereomeric forms, or mixtures thereof. The compounds of formula (I) or formula (II) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formula (I) or formula (II), but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts or solvates of said compounds which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) or formula (II) of this invention thereof can generally be prepared by carrying out the procedures disclosed in the Schemes and/or in the Examples below, by substituting a readily available isotopically labeled reagent for a non-isotopically labeled reagent.

Other aspects, advantages, and features of the invention will become apparent from the detailed description below.

### DETAILED DESCRIPTION AND EMBODIMENTS OF THE INVENTION

The following reaction Schemes illustrate the preparation of the compounds of the present invention. Unless otherwise indicated, R¹—R²⁴ in the reaction schemes and the discussion that follows are as defined above.

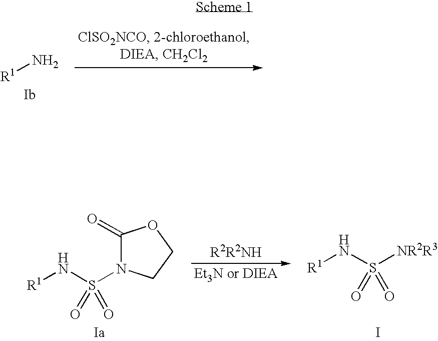

Referring to Scheme 1, the compound of formula Ia can be prepared by first reacting chlorosulfonyl isocyanate and 2-chloroethanol at low temperature such as in the range of about 0° C. to about 5° C. for a time between about 0.25 h to about 2 h, followed by the addition an amine of formula Ib with a suitable base such as triethylamine or diisopropylethylamine in a suitable solvent such as dichloromethane at a temperature of about 0° C. to about 5° C., and stirring is continued at temperature of about 0° C. for a time between about 0.5 h to about 2 h, and then at a temperature of about 25° C. from at a time between about 0 h to about 24 h. The compound of formula I can be prepared by reacting the compound of formula Ia with R²R³NH (1-2 equiv) in the presence of a suitable base such as triethylamine or diisopropylethylamine (2-3 equiv) in a suitable solvent such as acetonitrile, DMF, or a mixture of acetonitrile and DMF. The reaction can be carried out at an elevated temperature between about 70° C. and about 100° C. overnight, or the reaction can be carried out at an elevated temperature between about 70° C. and about 140° C. for about 10 min to about 2 h in a Personal Chemistry Smithsynthesizer® microwave, commercially available from Personal Chemistry of Uppsala, Sweden. R¹ is selected from the group consisting of —(CR³R⁴)ₜ(C₃-C₁₂)cycloalkyl, —(CR⁴R⁵)ₜ(C₆-C₁₂)aryl, and —(CR⁴R⁵)ₜ(4 to 10-membered heterocyclyl. R² and R³ are defined as above.

Any of the above compounds of formula I, Ia, and Ib, can be converted into another analogous compound by standard chemical manipulations. All starting materials, regents, and solvents are commercially available and are known to those of skill in the art unless otherwise stated. These chemical manipulations are known to those skilled in the art and include (a) removal of a protecting group by methods outlined in T. W. Greene and P. G. M. Wuts, Protective Groups in Organic Svnthesis, 2nd Ed., John Wiley and Sons, New York, 1991; (b) displacement of a leaving group (halide, mesylate, tosylate, etc) with a primary or secondary amine, thiol or alcohol to form a secondary or tertiary amine, thioether or ether, respectively; (c) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; (d) reductive amination of a primary or secondary amine using an aldehyde.

The compounds of the present invention may have asymmetric carbon atoms. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formula (I) or formula (II) that are basic in nature are capable of forming a wide variety of different salts with various inorganic and organic acids. Although such salts must be pharmaceutically acceptable for administration to animals, it is often desirable in practice to initially isolate the compound of formula (I) or formula (II) from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula (I) or formula (II) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula (I) or formula (II). Such non-toxic base salts include those derived from such pharmacologically acceptable cations as sodium, potassium, calcium, and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention may be modulators of 11-β-hsd-1. The compounds of the present invention may modulate processes mediated by 11-β-hsd-1, which refer to biological, physiological, endocrinological, and other bodily processes which are mediated by receptor or receptor combinations which are responsive to the 11-β-hsd-1 inhibitors described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g.) brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such

processes can be accomplished in vitro or in vivo. In vivo modulation can be carried out in a wide range of subjects, such as, for example, humans, rodents, sheep, pigs, cows, and the like.

The compounds according to the present invention may be used in several indications which involve modulations of 11-β-hsd-1 enzyme. Thus, the compounds according to the present invention may be used against dementia (See WO97/07789), osteoporosis (See Canalis E 1996, “Mechanisms of Glucocorticoid Action in Bone: Implications to Glucocorticoid-Induced Osteoporosis”, Journal of Clinical Endocrinology and Metabolism, 81, 3441-3447) and may also be used disorders in the immune system (see Franchimont, et. al, “Inhibition of Th1 Immune Response by Glucocorticoids: Dexamethasone Selectively Inhibits IL-12-induced Stat 4 Phosphorylation in T Lymphocytes”, The Journal of Immunology Feb. 15, 2000, vol 164 (4), p. 1768-74) and also in the above listed indications.

Inhibition of 11-β-hsd-1 in isolated murine pancreatic β-cells improves the glucose-stimulated insulin secretion (Davani, B., et al. (2000) J. Biol. Chem. Nov. 10, 2000; 275(45): 34841-4). Glucocorticoids were previously known to reduce pancreatic insulin release in vivo (Billaudel, B. and B. C. J. Sutter (1979) Horm. Metab. Res. 11: 555-560). Thus, inhibition of 11-β-hsd-1 is predicted to yield other beneficial effects for diabetes treatment, besides effects on liver and fat.

Recent data suggests that the levels of the glucocorticoid target receptors and the 11-β-hsd-1 enzymes determine the susceptibility to glaucoma (Stokes, J., et al., (2000) Invest. Ophthalmol. 41:1629-1638). Further, inhibition of 11-β-hsd-1 was recently presented as a novel approach to lower the intraocular pressure (Walker E. A., et al, poster P3-698 at the Endocrine society meeting Jun. 12-15, 1999, San Diego). Ingestion of carbenoxolone, a non-specific inhibitor of 11-β-hsd-1, was shown to reduce the intraocular pressure by 20% in normal subjects. In the eye, expression of 11-β-hsd-1 is confined to basal cells of the corneal epithelium and the non-pigmented epithelialium of the cornea (the site of aqueous production), to ciliary muscle and to the sphincter and dilator muscles of the iris. In contrast, the distant isoenzyme 11 beta-hydroxysteroid dehydrogenase type 2 is highly expressed in the non-pigmented ciliary epithelium and corneal endothelium. None of the enzymes is found at the trabecular meshwork, the site of drainage. Thus, 11-β-hsd-1 is suggested to have a role in aqueous production, rather than drainage, but it is presently unknown if this is by interfering with activation of the glucocorticoid or the mineralocorticoid receptor, or both.

Bile acids inhibit 11-β-hydroxysteroid dehydrogenase type 2. This results in a shift in the overall body balance in favor of cortisol over cortisone, as shown by studying the ratio of the urinary metabolites (Quattropani C, Vogt B, Odermaft A, Dick B, Frey B M, Frey F J. 2001. “Reduced Activity of 11-beta-hydroxysteroid dehydrogenase in Patients with Cholestasis,” J Clin Invest. November; 108(9): 1299-305). Reducing the activity of 11-β-hsd-1 in the liver by a selective inhibitor is predicted to reverse this imbalance, and acutely counter the symptoms such as hypertension, while awaiting surgical treatment removing the biliary obstruction.

The compounds of the present invention may also be useful in the treatment of other metabolic disorders associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and

many other conditions linked to NIDDM, including dyslipidemia glucocorticoid induced insulin resistance, dyslipidemia, polycysitic ovarian syndrome, obesity, hyperglycemia, hyperlipidemia, hypercholesteremia, hypertnglyceridemia, hyperinsulinemia, and hypertension. Brief definitions of these conditions are available in any medical dictionary, for instance, Stedman's Medical Dictionary (10^{th} Ed.).

### Assay

The 11β-hsd-1 assay was performed in a 100 mM Triethanolamine buffer pH 8.0, containing 200 mM NaCl, 0.02% n-dodecyl β-D-maltoside, 5% glycerol, 5 mM β-mercaptoethanol. A typical reaction for the determination of Kᵢₐₚₚ values was carried at R.T. in a Corning® u-bottom 96-well plate and is described as follows: 11β-hsd-1 enzyme (5 nM, final concentration) was pre-incubated in the presence of the inhibitor and NADPH (500 μM, final concentration) for at least 30 minutes in the assay buffer. When pre-incubation was completed, the reaction was initiated by adding the regenerating system (2 mM Glucose-6-Phosphate, 1U/mL Glucose-6-Phosphate dehydrogenase, and 6 mM MgCl₂, all the concentration reported are final in the assay buffer), and 3H-cortisone (200 nM, final concentration). After 60 minutes, 60 μL of the assay mixture was transferred to a second 96-well plate and mixed with an equal volume of dimethylsulfoxide to stop the reaction. A 15 μL aliquot from the reaction mixture was loaded into a C-18 column (Polaris C18-A, 50×4.6 mm, 5 μ, 180 Angstrom from Varian) connected to an automated High-throughput Liquid Chromatography instrument developed by Cohesive Technologies Inc, of Franklin, Mass. USA, with a β-RAM model 3 Radio-HPLC detector from IN/US, of Tampa, Fla. USA. The substrate and product peaks were separated by using an isocratic mixture of 43:57 methanol to water (v/v) at a flow rate of 1.0 mL/min.

The initial reaction velocities were measured by stopping the reaction at 60 min and by measuring the area of product formation in the absence and the presence of various concentrations of inhibitors. The Kᵢₐₚₚ values were determined using the equation for tight-binding inhibitor developed by Morrison, J. F. (Morrison J. F. Biochim BiophysActa. 1969; 185: 269-86).

The radiolabeled [1,2-3H]-cortisone is commercially available from American Radiolabeled Chemicals Inc of St. Louis, Mo. USA. NADPH, Glucose-6-Phosphate, and Glucose-6-Phosphate dehydrogenase were purchased from Sigma®.

The Kᵢₐₚₚ values of the compounds of the present invention for the 11-β-hsd-1 enzyme may lie typically between about 10 nM and about 10 μM. The compounds of the present invention that were tested all have Kᵢₚₚ's in at least one of the above HPLC-based assays of less than 1 μM, preferably less than 100 nM. Certain preferred groups of compounds possess differential selectivity toward the various 11-β-hsd's. One group of preferred compounds possesses selective activity towards 11-β-hsd-1 over 11β-hsd-2. Another preferred group of compounds possesses selective activity towards 11β hsd-2 over 11-β-hsd-1. (Morrison J. F. Biochim Biophys Acta. 1969; 185: 269-86).

Percentage of inhibition was determined in a 100 mM Triethanolamine buffer, pH 8.0, 200 mM NaCl, 0.02% n-dodecyl β-D-maltoside and 5 mM β-ME. A typical reaction was carried on a Corning® u-bottom 96-well plate and is described as follows: 11β-hsd-1 enzyme (5 nM, final concentration) was pre-incubated in the presence of the inhibitor and NADPH (500 μM, final concentration) for at least 30 minutes

in the assay buffer. When pre-incubation was completed, the reaction was initiated by adding the regenerating system (2 mM Glucose-6-Phosphate, 1U/mL Glucose-6-Phosphate dehydrogenase, and 6 mM MgCl₂, all the concentration reported are final in the assay buffer), and 3H-cortisone (200 nM, final concentration). After 60 minutes, 60 μL of the assay mixture was transferred to a second 96-well plate and mixed with an equal volume of dimethylsulfoxide to stop the reaction. A 15 μL aliquot from the reaction mixture was loaded into a C-18 column (Polaris C18-A, 50×4.6 mm, 5 μ, 180 Angstrom from Varian) connected to an automated High-throughput Liquid Chromatography instrument developed by Cohesive Technologies Inc of Franklin, Mass., with a 3-RAM model 3 Radio-HPLC detector from IN/US of Tampa, Fla. The substrate and product peaks were separated by using an isocratic mixture of 43:57 methanol to water (v/v) at a flow rate of 1.0 mL/min.

Percent Inhibition was calculated based on the following equation: (1-(3H-Cortisol peak area in the absence of inhibitor/3H-cortisol peak area in the presence of inhibitor)×100). Certain groups of compounds possess differential selectivity toward the various 11-β-hsd enzymes. One group of compounds possesses selective activity towards 11-β-hsd-1enzyme over 11β-hsd-2 enzyme. While another group of compounds possesses selective activity towards 11βhsd-2 enzymes over 11-β-hsd-1 enzymes.

### Pharmaceutical Compositions/Formulations, Dosaging and Modes of Administration

Methods of preparing various pharmaceutical compositions with a specific amount of active compound are known, or will be apparent, to those skilled in this art. In addition, those of ordinary skill in the art are familiar with formulation and administration techniques. Such topics would be discussed, e.g. in Goodman and Gilman's The Pharmaceutical Basis of Therapeutics, current ed., Pergamon Press; and Remington's Pharmaceutical Sciences, current ed., Mack Publishing, Co., Easton, Pa. These techniques can be employed in appropriate aspects and embodiments of the methods and compositions described herein. The following examples are provided for illustrative purposes only and are not meant to serve as limitations of the present invention.

The compounds of formula (I), (II) and (III) may be provided in suitable topical, oral and parenteral pharmaceutical formulations for use in the treatment of 11-β-hsd-1 mediated diseases. The compounds of the present invention may be administered orally as tablets or capsules, as oily or aqueous suspensions, lozenges, troches, powders, granules, emulsions, syrups or elixirs. The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occurring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to know methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the acceptable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The compounds of formula (I), (II) and (III) may also be administered in the form of suppositories for rectal administration of the drug. These compositions can be prepared by mixing the drug with a suitable non-irritating excipient that is solid at about 25 Celsius but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The compounds of formula (I), (II) and (III) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. A preferred dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered

concomitantly with other orally active antidiabetic compounds such as the sulfonylureas, for example, tolbutamide and the like.

For administration to the eye, a compound of the present invention is delivered in a pharmaceutically acceptable ophthalmic vehicle such that the compound is maintained in contact with the ocular surface for a sufficient time period to allow the compound to penetrate the cornea and/or sclera and internal regions of the eye, including, for example, the anterior chamber, posterior chamber, vitreous body, aqueous humor, vitreous humor, cornea, iris/ciliary's, lens, choroid/retina and sclera. The pharmaceutically acceptable ophthalmic vehicle may be an ointment, vegetable oil, or an encapsulating material. A compound of the invention may also be injected directly into the vitreous humor or aqueous humor.

Further, a compound may be also be administered by well known, acceptable methods, such as subtenon and/or subconjunctival injections. As is well known in the ophthalmic art, the macula is comprised primarily of retinal cones and is the region of maximum visual acuity in the retina. A Tenon's capsule or Tenon's membrane is disposed on the sclera. A conjunctiva covers a short area of the globe of the eye posterior to the limbus (the bulbar conjunctiva) and folds up (the upper cul-de-sac) or down (the lower cul-de-sac) to cover the inner areas of the upper eyelid and lower eyelid, respectively. The conjunctiva is disposed on top of Tenon's capsule. The sclera and Tenon's capsule define the exterior surface of the globe of the eye. For treatment of age related macular degeneration (ARMD), choroid neovascularization, retinopathies (such as diabetic retinopathy, retinopathy of prematurity), retinitis, uveitis, cystoid macular edema (CME), glaucoma, and other diseases or conditions of the posterior segment of the eye, it is preferable to dispose a depot of a specific quantity of an ophthalmically acceptable pharmaceutically active agent directly on the outer surface of the sciera and below Tenon's capsule. In addition, in cases of ARMD and CME it is most preferable to dispose the depot directly on the outer surface of the sclera, below Tenon's capsule, and generally above the macula.

The compounds may be formulated as a depot preparation. Such long-acting formulations may be administered by implantation (for example, subcutaneously or intramuscularly) intramuscular injection or by the above mentioned subtenon or intravitreal injection. Alternatively, the active ingredient may be in powder form for constitution with a suitable vehicle, e.g., sterile pyrogen-free water, before use.

Within particularly preferred embodiments of the invention, the compounds may be prepared for topical administration in saline (combined with any of the preservatives and antimicrobial agents commonly used in ocular preparations), and administered in eyedrop form. The solution or suspension may be prepared in its pure form and administered several times daily. Alternatively, the present compositions, prepared as described above, may also be administered directly to the cornea.

Within preferred embodiments, the composition is prepared with a muco-adhesive polymer which binds to cornea. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other-protonic solvents than are the corresponding free-base forms.

The preparation of preferred compounds of the present invention is described in detail in the following examples, but the artisan will recognize that the chemical reactions described may be readily adapted to prepare a number of other compounds of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having applicability for preparing other compounds of the invention.

### EXAMPLES

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

The structures of the compounds are confirmed by either elemental analysis or NMR, where peaks assigned to the characteristic protons in the title compound are presented where appropriate. ¹H NMR shift (δ_{H}) are given in parts per million (ppm) down field from an internal reference standard.

The invention will now be described in reference to the following examples. These examples are not to be regarded as limiting the scope of the present invention, but shall only serve in an illustrative manner.

Method A

The following examples illustrate Method A by which compounds of formula (I) and (III) of the invention can be prepared. Additional examples prepared by Method A are shown in Table 1 below.

### Example 1

### N-(4,6-dimethylpyridin-2-yl)-4-[5-(trifluoromethyl)pyridin-2-yl]piperazine-1-sufonamide

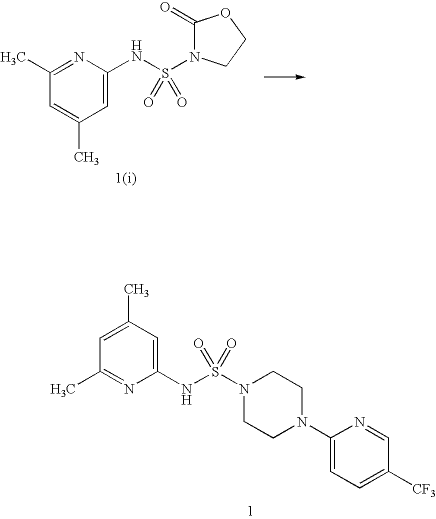

### Intermediate 1(i): N-(4,6-dimethylpyridin-2-yl)-2-oxo-1,3-oxazolidine-3-sulfonamide

Chlorosulfonyl isocyanate (1.24 mL, 14.3 mmol) was added to an ice-cooled solution of dichloromethane (25 mL). 2-Chloroethanol (0.96 mL, 14.3 mmol) was then added via syringe over one minute to maintain an internal temperature of less than 2° C. After the reaction mixture was stirred for 0.5 h, N,N-diisopropylethylamine (7 mL, 43 mmol) was added. A solution of 2-amino-4,6-dimethylpyridine (1.75 g, 14.3 mmol) in dichloromethane (25 mL) was added dropwise over 5 minutes, then stirred for an additional 0.5 h at 0° C. The reaction was quenched by adding 0.2 N HCl (50 mL) and dichloromethane. The organic layer was separated and concentrated via in vacuo distillation. The residue was triturated with water (30 mL) and then concentrated. The residue was then diluted with 20 mL of dichloromethane to form a white solid, the solid was filtered and rinsed with an additional 5 mL of dichloromethane to afford product 1(i) as a white solid after drying under high vacuum (3.46 g, 95%). ¹H NMR (400 MHz, CDCl₃),δ: 6.94 (s, 1 H), 6.47 (s, 1 H), 4.32 (t, J=5.3, 2 H), 3.64 (t, J=5.6 Hz, 2 H), 2.48 (s, 3 H), 2.34 (s, 3 H); LCMS (ESI): 272.0.

### Example 1

### N-(4,6-dimethylpyridin-2-yl)-4-[1-(trifluoromethyl)pyridin-2-yl]piperazine-1-sulfonamide

Intermediate 1(i) (N-(4,6-dimethylpyridin-2-yl)-2-oxo-1,3-oxazolidine-3-sulfonamide (0.23 g, 0.84 mmol), 1-[5-(trifluoromethyl)pyridin-2-yl]piperazine) (0.34 g, 1.017 mmol) and DIEA (1.38 mL, 8.48 mmol) were dissolved in acetonitrile (2 mL). The reaction mixture was warmed to 130° C. using a Smithsynthesizer® microwave heating for 0.5 h then cooled to 25° C. The reaction mixture was diluted with 50 mL of ethyl acetate and washed twice with 30 mL of aqueous saturated ammonium chloride and washed twice with 30 mL of aqueous saturated sodium bicarbonate. The organic layer was separated and concentrated in vacuo to give a clear oil. The residue was purified using silica gel flash chromatography eluting with hexane/ethyl acetate (1:1) and the product containing fractions were collected and concentrated. The residue was triturated twice with 2 mL of ethyl acetate, filtered and dried on high vacuum to afford the product 1 as a white solid (0.25 g, 70%). See Table 1 below for ¹H NMR.

### Example 12

### 7-(Methylsulfonyl)-N-quinolin-2-yl-1,2,4,5-tetrahydro-3H-3-benzazepine-3-sulfonamide

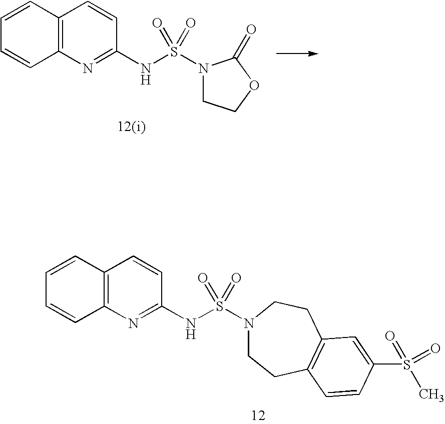

### Intermediate 12(i): 2-oxo-N-quinolin-2-yl-1,3-oxazolidine-3-sulfonamide

Chlorosulfonyl isocyanate (0.37 mL, 4.1 mmol) was dissolved in CH₂Cl₂ (40 mL) and cooled to 0° C. Chloroethanol (0.27 mL, 4.1 mmol) was then slowly added and the reaction solution was stirred at 0° C. for 1.5 h. A solution of aminoquinoline (4.1 mmol) and triethylamine (12.4 mmol) in CH₂Cl₂ (50 mL) was slowly added to the reaction solution that the reaction temperature did not exceed 5° C. The reaction solution was slowly warmed to room temperature and stirred overnight. The reaction solution was first concentrated in vacuo. The residue was dissolved in CH₂Cl₂ (200 mL), washed with 1N HCl (15 mL), brine (15 mL), dried over MgSO₄ and concentrated in vacuo. The product 12(i) was recrystallized from CH₂Cl₂ to afford the desired product. ¹H NMR (400 MHz, CD₃CN), 5 ppm 4.02-4.09 (m, 2 H) 4.33-4.41 (m, 2 H) 7.20 (d, J=9.35 Hz, 1 H) 7.50 (t, J=7.58 Hz, 1 H) 7.62 (d, J=8.34 Hz, 1 H) 7.72-7.79 (m, 1 H) 7.86 (d, J=7.83 Hz, 1 H) 8.22 (d, J=9.35 Hz, 1 H) 11.74 (b, 1H); APCIMS: m/z 294.1 (M+1).

### Example 12

### 7-(Methylsulfonyl)-N-quinolin-2-yl-1,2,4,5tetrahydro-3H-3-benzazepine-3-sulfonamide

The title compound was made using a method analogous to that described for Example 1 above. Intermediate 12(i) (2-oxo-N-quinolin-2-yl-1,3-oxazolidine-3-sulfonamide) (0.5 mmol) and 7-(methylsulfonyl)-2,3,4,5-tetrahydro-1H-3-benzazepine (0.6 mmol) [Macdonald, G. J. et al. J. Med.Chem. 2003, 46, 4952] were placed in a microwave tube and acetonitrile (2 mL) and triethylamine (0.21 mL) were then added. The reaction was carried out at 140° C. for 10 min. After the solution was cooled to room temperature, the reaction mixture was diluted with EtOAc (100 mL), washed with saturated sodium bicarbonate (10 mL), brine (twice using 10 mL), and dried over MgSO₄. The organic layer was concentrated in vacuo to give the crude product 12 which was purified by prep-TLC eluting with 5% MeOH in CH₂Cl₂ to afford the desired product (108 mg, 50%). See Table 1 below for ¹H NMR.

### Example 15

### Ethyl {6-[(1,2,4,5-tetrahydro-3H-3-benzazepin-3-ylsulfonyl)amino]pyridin-2-yl}acetate

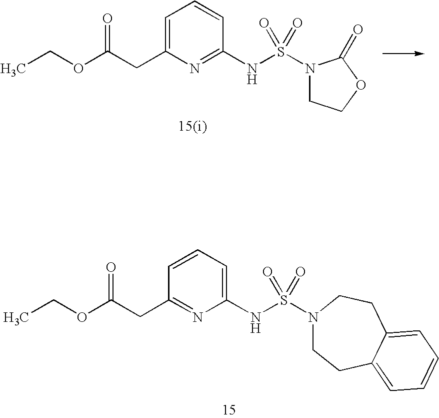

### Intermediate 15(i): Ethyl (6-{[(2-oxo-1,3-oxazolidin-3-yl)sulfonyl]amino}pyridin-2-yl)acetate

To a flask containing anhydrous CH₂Cl₂ (15 mL) under nitrogen at approximately 0° C. was added chlorosulfonyl isocyanate (0.8 mL) followed by dropwise addition of 2-chloroethanol (0.62 mL). After stirring at 5° C. for 100 min, a solution of ethyl (6-aminopyridine-2-yl)acetate.HCl (2.0 g) and TEA (5.1 mL) in anhydrous CH₂Cl₂ (75 mL) were slowly added to keep the internal temperature at about 5° C. At the end of the addition, the reaction mixture was allowed to warm to room temperature slowly. After stirring at room temperature for 15 h, the reaction mixture was concentrated under reduced pressure. The residue was dissolved in CH₂Cl₂ (1200 mL), washed with dilute HCl solution (18 mL of 1N HCl in 102 mL of water), brine, dried with K₂CO₃, filtered and concentrated under reduced pressure to give the desired product 15(i) as a foam weighing 2.3 g (76%). ¹H NMR (400 MHz, CDCl₃) δ ppm 1.21 (t, J=7.20 Hz, 3 H) 3.25-3.30 (m, 2 H) 3.65 (s, 2 H) 4.08-4.18 (m, 2 H) 4.25-4.35 (m, 2 H) 6.69 (d, J=7.33 Hz, 1 H) 6.94 (d, J=8.34 Hz, 1 H) 7.49 (t, J=7.96 Hz, 1 H)

### Example 15

### Ethyl {6-[(1,2,4,5-tetrahydro-3H-3-benzazepin-3-ylsulfonyl)amino]pyridin-2-yl}acetate

The title compound 15 was made using a method analogous to that described for Example 1 above, using instead intermediate 15(i) (ethyl (6-{[(2-oxo-1,3-oxazolidin-3-yl)sulfonyl]amino}pyridin-2-yl)acetate) (1.229 g), 2,3,4,5-tetrahydro-1H-3-benzazepine.HCl (752 mg), DIEA (1.01 mL), anhydrous DMF (4 mL). After heating at 100° C. for 50 min, the reaction mixture was concentrated to a solid under reduced pressure. CH₂Cl₂ (400 mL) was added to the reaction residue, and the resulting solution was washed with diluted HCl solution (twice using 3 mL of 1N HCl with 17 mL of water), brine, dried with K₂CO₃, filtered and concentrated under reduced pressure. The crude mixture was purified by silica gel chromatography eluting with MeOH:CH₂Cl₂ to give desired product (330 mg) and a mixture containing desired product (720 mg). After purification this mixture further purified under reversed phase column, the title compound 15 was obtained (513 mg, 35%). APCIMS: m/z 348.2 (M+1). See Table 1 below for ¹H NMR.

### Example 19

### N-(6-aminopyridin-2-yl)-4-(5-cyanopyridin-2-yl)piperazine-1-sulfonamide

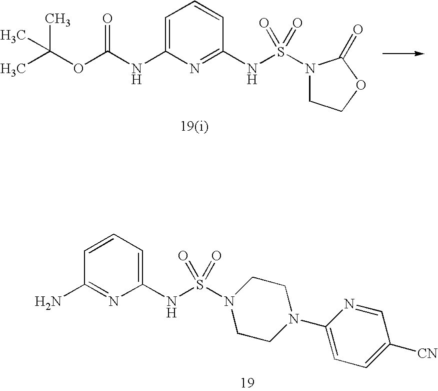

### Intermediate 19(i): Tert-butyl (6-{[2-oxo-1,3-oxazolidin-3-yl)suffonyl]amino}pyridin-2-yl)carbamate

The title compound 19(i) was made using a method analogous to that described for intermediate 1(i) above, using instead tert-butyl (6-aminopyridin-2-yl)carbamate (Berl, et al. Chem Eur J 2001, 7, 2798). ¹H NMR (400 MHz, CD₂Cl₂), δ: 1.50 (s, 9 H), 4.05-4.11 (m, 2H) 4.24-4.30 (m, 2H) 6.64 (d, J=7.83 Hz, 1 H) 7.32 (d, J=8.08 Hz, 1 H) 7.50 (t, J=8.08 Hz, 1H).

### Example 19

### N-(6-aminopyridin-2-yl)-4-(5-cyanopyridin-2-yl)piperazine-1-sulfonamide

The title compound 19 was synthesized using a method analogous to Example 1 above, using instead intermediate 19(i) (tert-butyl (6-{[(2-oxo-1,3-oxazolidin-3-yl)sulfonyl]amino}pyridin-2-yl)carbamate) and 6-piperazin-1-ylnicotinonitrile. LC-MS (APCI+) for C₁₅H₁₇N₇O₂S m/z 360.1 (M+H)⁺; t_{R}=2.393 min. See Table 1 below for ¹H NMR.

### Example 20

### N-(6-amino-4-methylpyridin-2-yl)-4-(5-cyanopyridin-2-yl)piperazine-1-sulfonamide

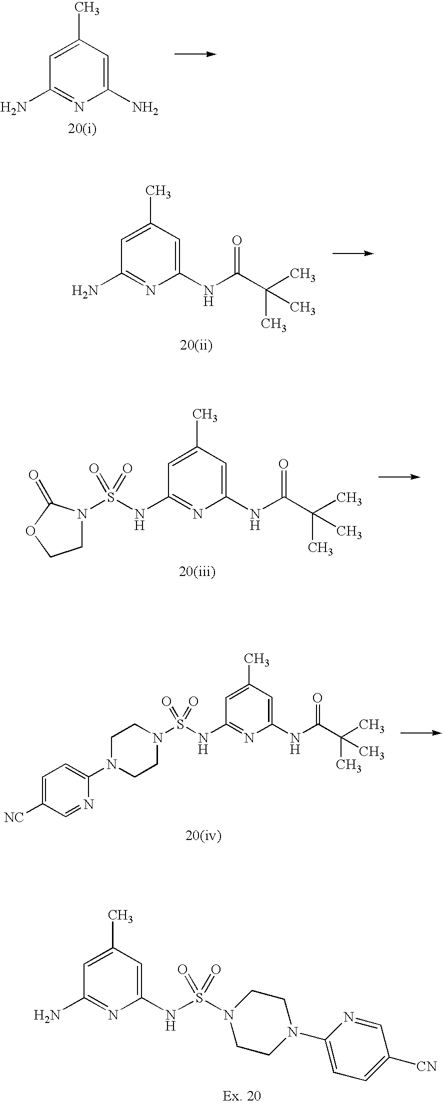

### Intermediate 20(i): 4-methylpyrddine-2,6-diamine

A solution of 4-methylpyridine (9.31 g, 0.10 mol) in tetralin (50 mL) was added to a solution of sodium amide (9.33 g, 0.24 mol) in tetralin (100 mL) at 130° C. After the addition, the mixture was heated to 195° C. and stirred for 10 h. The mixture was cooled to room temperature and filtered. The filter cake was added to ethanol (150 mL) in portions and the mixture was stirred for 30 min. The mixture was evaporated under reduced pressure to give crude which was pre-purified via column chromatography (silica gel, ethyl acetate) and then re-crystallized from CH₂Cl₂ to yield title compound 20(i) (2 g, 16.3%) as a white solid. ¹H NMR (400 MHz, DMSO-d₆) δ ppm 1.92 (s, 3 H) 5.17(s, 1 H) 5.43 (s, 1 H).

### Intermediate 20(ii): N-(6-amino-4-methylpyridin-2-yl)-2,2-dimethylpropanamide

To a stirred mixture of compound 20(i) (4-methylpyridine-2,6-diamine) (2 g, 0.016 mol), Et₃N (1.78 g, 0.176 mol) and anhydrous THF (20 mL) was added pivaloyl chloride dropwise (1.96 g, 0.016 mol) at 0° C. After the addition, the mixture was stirred at room temperature for 12 h. The reaction was quenched by addition of water (30 mL). The mixture was concentrated in vacuo to remove THF, and the residue was extracted with CH₂Cl₂ (2×40 mL). The combined organic layers were washed with saturated aqueous sodium bicarbonate (20 mL), dried over sodium sulfate and concentrated in vacuo. The residue was washed with hexane to give title compound 20(i) (1.8 g, 54%) as a white solid. ¹H NMR (400 MHz, CDCl₃) δ ppm 1.30 (s, 9H), 2.21 (s, 3 H) 4.23 (s, 2H) 6.08 (s, 1 H) 7.46 (s, 1 H).

### Intermediate 20(iii): 2,2-dimethyl-N-(4-methyl-6-{[(2-oxo-1,3-oxazolidin-3-yl)sulfonyl]amino}pyridin-2-yl)propanamide

To a mixture of 1,3-oxazolidin-2-one (43.5 g, 0.5 mol), Et₃N (60.5 g 0.5 mol), 4-(N,N-dimethylamino)pyridine (6.1 g, 0.05 mol) and anhydrous CH₂Cl₂ (200 mL) was added sulfuryl chloride dropwise (67.5 g, 0.5 mol) at 0° C. After the addition, the resulting mixture was stirred at 0° C. for 0.5 h. A solution o 20(ii) (N-(6-amino-4-methylpyridin-2-yl)-2,2-dimethylpropanamide) (52 g, 0.25 mol) in anhydrous CH₂Cl₂ (100 mL) was added dropwise at 0° C. The resulting mixture was stirred at 0° C. for 1 h and the reaction was quenched by addition of water (100 mL) and CH₂Cl₂ (200 mL). The organic layer was separated and washed with brine (100 mL×2), dried over sodium sulfate and concentrated in vacuo to give crude which was pre-purified via column chromatography (silica gel, CH₂Cl₂/Petroleum ether 1:1), and the product was washed with THF and dried to yield title product 20(iii) (8 g, 8.4%) as a white solid. ¹H NMR (400 MHz, CDCl₃) δ ppm 1.30 (s, 9H) 2.30(s, 3 H) 4.11 (m, 2H) 4.38 (m, 2H) 6.82 (s, 1 H) 7.30 (s, 1 H) 8.63 (s,1 H).

### Intermediate 20(iv): N-[6-({[4-(5cyanopyridin-2-yl)piperazin-1-yl]sulfonyl}amino)-4-methylpyridin-2-yl]-2,2-dimethyl-propanamide

Intermediate 20(iv) was made using a method analogous to that described for Example 1 above, except using instead intermediate 20(iii) (2,2-dimethyl-N-(4-methyl-6-{[(2-oxo-1,3-oxazolidin-3-yl)sulfonyl]amino}pyridin-2-yl)propanamide) and 6-piperazin-1-ylnicotinonitrile. ¹H NMR (400 MHz, CDCl₃) δ ppm 1.31 (s, 9 H) 2.35 (s, 3 H) 3.35-3.47 (m, 4 H) 3.68-3.79 (m, 4 H) 6.61 (d, J=9.09 Hz, 1 H) 6.83 (s, 1 H) 6.97 (s, 1 H) 7.65 (dd, J=9.09, 2.27 Hz, 1 H) 7.71 (s, 1 H) 7.79 (s, 1 H) 8.41 (d, J=2.02 Hz, 1 H).

### Example 20

### N-(6-amino-4-methylpyridin-2-yl)-4-(5-cyanopyridin-2-yl)piperazine-1-sulfonamide

Intermediate 20(iv) (N-[6-({[4-(5-cyanopyridin-2-yl)piperazin-1-yl]sulfonyl}amino)-4-methylpyridin-2-yl]-2,2-dimethyl-propanamide) (200 mg, 0.437 mmol) was dissolved in 6 N HCl (2 mL) solution and was heated to 80° C. After 10 h, the reaction mixture was cooled to RT and neutralized to pH 6 using 20% NaOH and saturated sodium bicarbonate and extracted with CH₂Cl₂ (4×50 mL). The layers were separated and the combined organic layer was dried over sodium sulfate. The crude product was concentrated in vacuo and triturated twice with MeOH/EtOAc/Hex to give the title compound 20 (52 mg, 32% yield). HRMS m/z calcd for (M+H)⁺374.1394, found 374.1396; Calcd for C₁₆H₂₀N₇O₂S: C, 51.36; H, 5.13; N, 26.26. Found: C, 51.26; H, 5.12; N, 26.05. See Table 1 below for ¹H NMR.

### Example 23

### (3R)-4-(5-cyanopyridin-2-yl)-3-methyl-N-(6-methylpyridin-2-yl)piperazine-1-sulfonamide

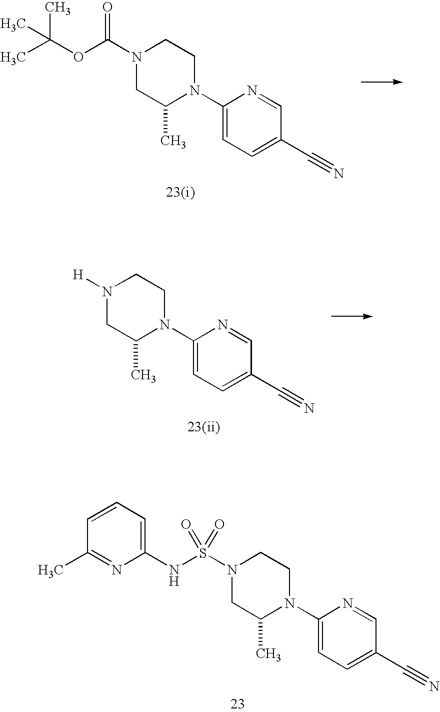

### Intermediate 23(i): tert-Butyl (3R)-4-(5-cyanopyridin-2-yl)-3-methylpiperazine-1-carboxylate

The compound tert-butyl (3R)-3-methylpiperazine-1-carboxylate (25 g, 124.8 mmol) was dissolved in DMSO (50 mL). To reaction mixture was added 6-chloronicotinonitrile (19 g, 137.3 mmol), K₂CO₃ (32.7 g, 235.9 mmol) and Cu(MeCN)₄PF₆ (0.42 g, 1.12 mmol) then warmed to 140° C. for 4 h. The solution was cooled to 25° C. and diluted with EtOAc (200 mL) then partitioned between aqueous HCl (3×50 mL 0.1 N) and saturated aqueous sodium bicarbonate (2×50 mL). The organic layer was dried over sodium sulfate, filtered through silica (50 mL) and concentrated. The residue was purified by crystallizing from 20 mL of warm EtOAc after standing for 2 h at 25° C. The mother liquor was decanted, the solid rinsed with EtOAc (2×10 mL) and placed under high vacuum for 2 h that afforded the title compound 23(i) as a white crystalline solid (32.6 g, 96%). HPLC Rt: 3.444 (95.4%). ¹H NMR (400 MHz, CDCl₃) δ: 8.42 (s, 1H), 7.63 (dd, J=9.1, 2.3 Hz, 1H), 6.56 (d, J=9.1 Hz, 1H), 5.30 (s, 1H), 4.53 (bs, 1H), 4.13 (bs, 1H), 3.94 (bs, 1H), 3.26-3.21 (m, 2H), 2.99 (bs, 1H), 1.49 (s, 9H), 1.20 (d, J=6.8 Hz, 3H). LCMS (ESI): m/z: 303.3.

### Intermediate 23(ii): 6-[(2R)-2-methylpiperazin-1-yl]nicotinonitrile hydrochloride

To intermediate 23(i) (tert-Butyl (3R)-4-(5-cyanopyridin-2-yl)-3-methylpiperazine-1-carboxylate) (32.0 g, 105.5 mmol) was added to 50 mL of 6N HCl and the mixture stirred at 90° C. for 1 h. The solution was cooled to 25° C., triturated with EtOAc (2×40 mL) and then concentrated. To the resultant gummy solid was added acetone (100 mL), slurred then filtered and dried under high vacuum to give afford title compound 23(ii) as a white crystalline solid (25.1 g, 100%). HPLC Rt: 1.547 (100%). ¹H NMR (400 MHz, D₂O) δ: 8.34 (s, 1H), 7.81 (dd, J=9.4, 2.3 Hz, 1H), 6.92 (d, J=8.9 Hz, 1H), 4.69 (bs, 1H), 4.27-4.20 (m, 1H), 3.45-3.37 (m, 2H), 3.33-3.23 (m, 2H), 3.09 (td, J=13.3, 4.3 Hz, 1H), 1.23 (d, J=7.3 Hz, 1H); LCMS (ESI): m/z: 203.1.

### Example 23

### (3R)-4-(5-cyanopyridin-2-yl)-3-methyl-N-(6-methylpyridin-2-yl)piperazine-1-sulfonamide

The title compound 23 was made using a method analogous to that described for Example 1 above, using instead intermediate 2(i) (N-(6-methylpyridin-2-yl)-2-oxo-1,3-oxazolidine-3-sulfonamide) and 23(i) (6-[(2R)-2-methylpiperazin-1-yl]nicotino-nitrile hydrochloride). See Table 1 below for ¹H NMR.

### Examples 25 and 26

### 4-(4-cyanophenyl)-4-fluoro-N-(6-methylpyridin-2-yl)piper-idine-1-sulfonamide (25) and 4-(4-cyanophenyl)-N-(6-methylpyridin-2-yl)-3,6-dihydro-pyridine-1(2H-sulfonamide (26)

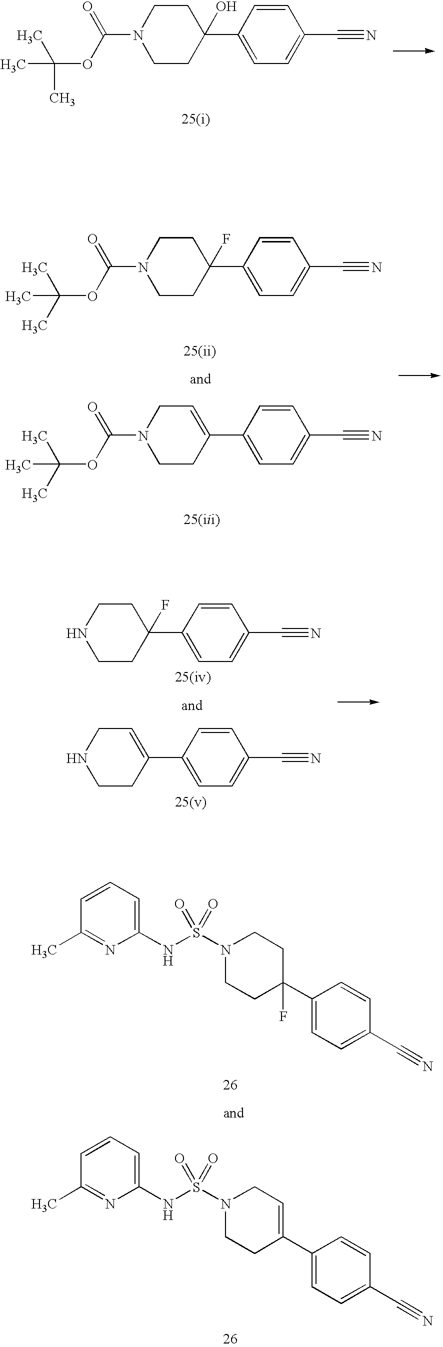

### Intermediate 25(i): Tert-butyl 4-(4-cyanophenyl)-4-hydroxypiperidine-1-carboxylate

A solution of 4-iodobenzonitrile (7.0 g, 30.5 mmol) in anhydrous THF (40 mL) was cooled to −40° C. using an acetonitrile/CO₂ bath. Isopropyl magnesium chloride (20 mL, 40 mmol, 2.0 M in THF) was then added over 20 min. and then stirred for 2 h. The compound tert-butyl 4-oxopiperidine-1-carboxylate (6.7 g, 33.6 mmol) was dissolved in THF (10 mL), then added dropwise to the reaction mixture and stirred for 1 h. Diluted mixture with ethyl acetate (50 mL), washed with aqueous HCl (0.1 N) and saturated aqueous sodium bicarbonate (2×30 mL), then dried organic layer over sodium sulfate and concentrated. The residue was purified using silica gel chromatography eluting with Hexane/EtOAc (2:1), combined purified fractions and concentrated to afford title compound 25(i) as a clear oil (7.0 g, 76%). HPLC Rₜ: 3.048 min. (80.2 %); ¹H NMR (400 MHz, CDCl₃) δ ppm: 1.47 (s, 9 H), 1.70 (d, J=13.64 Hz, 2 H), 1.75 (s, 1 H), 1.96 (s, 2 H), 3.22 (bs., 2 H), 4.06 (s, 2 H), 7.59-7.70 (m, 4 H); LRMS (ESI): m/z (M+H, -BOC): 203.2.

### Intermediate 25(ii): Tert-butyl 4-(4-cyanophenyl)-4-fluoropiperidine-1-carboxylate and tert-butyl 4-(4-cyanophenyl)-3,6-dihydropyridine-1 (2H)-carboxylate

The intermediate 25(i) (tert-butyl 4-(4-cyanophenyl)-4-hydroxypiperidine-1-carboxylate) (2.2 g, 7.3 mmol) was dissolved in dichloromethane (15 mL) and cooled to −40° C. To the solution was added DAST (1.4 g, 8.7 mmol) dropwise over 10 min. then warmed to 0° C. and stirred for 1 h. The mixture was quenched with saturated aqueous ammonium chloride (5 mL) and diluted with ethyl acetate (30 mL). The organic layer was partitioned between saturated aqueous sodium bicarbonate (2×30 mL), filtered through silica gel (15 mL) and the organic layer was concentrated to give clear oil. The residue was purified using silica gel chromatography eluting with hexane/ethyl acetate (2:1) and the purified fractions were combined to afford mixture of 25(ii) (tert-butyl 4-(4-cyanophenyl)-4-fluoropiperidine-1-carboxylate) and 25(iii) (tert-butyl 4-(4-cyanophenyl)-3,6-dihydropyridine-1(2H)-carboxylate) as a clear oil (0.48 g, approx. 63%). HPLC Rₜ: 3.819 min. (85%, a mixture of products); LRMS (ESI): m/z (M+H): 305.2, 286.1.

### Intermediates 25(iv) and 25(v): 4-(4-fluoropiperidin-4-yl)benzonitrile & 4-(1,2,3,6-tetrahydropyridin-4-yl)benzonitrile

The title compoundw were made by dissolving a mixture of 25(ii) (tert-butyl 4-(4-cyanophenyl)-4-fluoropiperidine-1-carboxylate) and 25(iii) (tert-butyl 4-(4-cyanophenyl)-3,6-dihydropyridine-1 (2H)-carboxylate) (0.85 g, 7.3 mmol) from previous step in dichloromethane (5 mL) and adding TFA (5 mL). The mixture was stirred for 1 h and azeotroped with toluene (2×10 mL) and then concentrated under high vacuum for 24 h and used without further purification to afford title products 25(iv) and 25(v) which were used without further purification (87%, a mixture of products). HPLC Rₜ: 1.545 min. LRMS (ESI): m/z (M+H): 185.2, 205.2.

### Examples 25 and 26

### 4-(4-cyanophenyl)-4-fluoro-N-(6-methylpyridin-2-yl)piper-idine-1-sulfonamide (25) and 4-(4-cyanophenyl)-N-(6-methylpyridin-2-yl)-3,6-dihydro-pyridine-1(2H)-sulfonamide (26)

The title compounds were made using a method analogous to that described for Example 1 above, using instead intermediate 2(i) (N-(6-methylpyridin-2-yl)-2-oxo-1,3-oxazolidine-3-sulfonamide) (0.17 g, 0.67 mmol), as prepared under Method B below, and a mixture of 25(iv) (4-(4-fluoropiperidin-4-yl)benzonitrile) and 25(v) (4-(1,2,3,6-tetrahydropyridin-4-yl)benzonitrile) (0.15 g, 0.74 mmol). The work up was done by diluting the reaction mixture with ethyl acetate (50 mL) and washing with saturated aqueous ammonium chloride (2×30 mL) and saturated sodium bicarbonate (2×50 mL). The organic layer was dried over sodium sulfate and concentrated. The resultant mixture was separated by flash column chromatography through silica gel eluting with hexanes: ethyl acetate (2:1) to afford title products as a white solid 25 (0.041, 16%) and foam 26 (0.021, 8.9%). See Table 1 below for ¹H NMR.

### Example 29

### 4-(5-cyanopyridin-2-yl)-N-[6-(hydroxymethyl)pyridin-2-yl]piperazine-1-sulfonamide

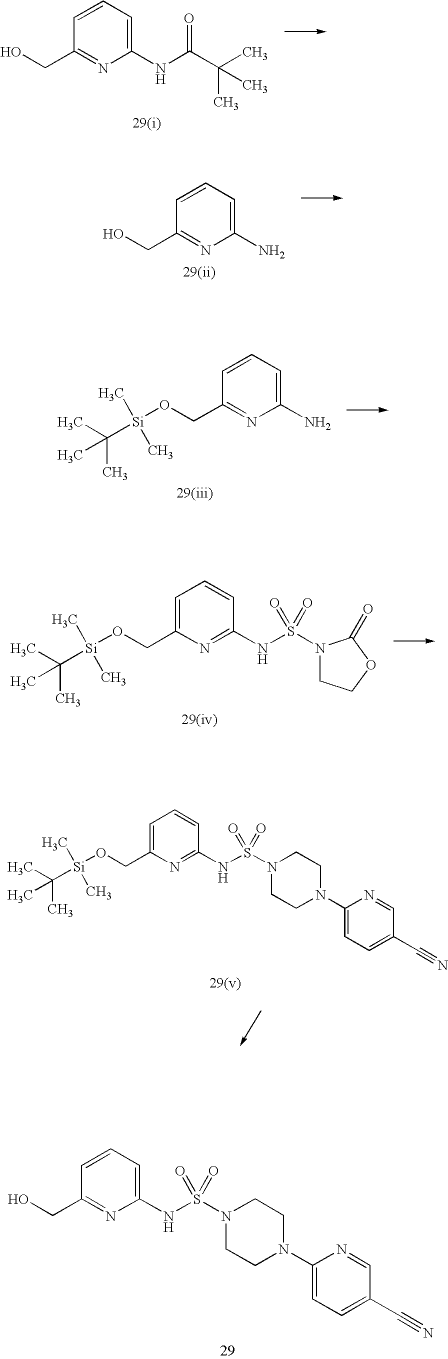

### Intermediate 29(i): N-[6-(1-Hydroxyethyl)pyridin-2-y-2,2-dimethylpropanamide

To solution of N-(6-formylpyridin-2-yl)-2,2-dimethylpropanamide (3.0 g, 14.9 mmol) in methanol (10 mL) was added sodium borohydride (1.37 g, 37.1 mmol) and stirred for 3 hours. The mixture diluted with ethyl acetate (50 mL). The mixture was washed with aqueous hydrochloric acid (2×30 mL, 0.1N) and saturated sodium bicarbonate (2×50 mL). The organic layer was dried over anhydrous Na₂SO₄, filtered, and concentrated to afford the title product 29(i) as a white solid (2.49 g, 80%). ¹H NMR (400 MHz, CDCl₃), δ: 8.16 (d, J=8.3 Hz, 1 H), 8.00 (br s, 1 H), 7.71 (t, J=7.8 Hz, 1 H), 7.12 (d, J=7.5 Hz, 1 H), 4.05-3.96 (m, 2 H), 1.35 (s, 9H); LRMS (ESI): m/z: 209.2.

### Intermediate 29(ii): 6-aminopyridin-2-yl-methanol

To a solution of dioxane (15 mL) was added intermediate 29(i) (N-[6-(1-hydroxyethyl)pyridin-2-yl]-2,2-dimethylpropanamide) (1.5 g, 7.2 mmol) and aqueous hydrochloric acid (6N, 15 mL) and the mixture was stirred at 90° C. for 14 h. The solution was cooled 0° C., triturated with diethyl ether (2×30 mL) and the aqueous layer was neutralized using sodium hydroxide to approximately pH 8. The mixture was diluted with chloroform/IPA (10:1, 50 mL). The mixture was washed with saturated brine solution (2×50 mL). The organic layer was dried over anhydrous Na₂SO₄, filtered, and concentrated to afford the title product 29(ii) as a white solid (0.86 g, 96%). HPLC: Rₜ 0.628 min. (99.5% area). ¹H NMR (400 MHz, CDCl₃), δ: 7.48 (t, J=7.4 Hz, 1 H), 6.66 (d, J=7.3 Hz, 1 H), 6.50 (d, J=8.4 Hz, 1 H), 4.58 (s, 2 H), 4.23 (bs, 2 H). LCMS (ESI): m/z: 125.2.

### Intermediate 29(iii): 6-({[tert-Butyl(dimethyl)silyl]oxy}methyl)pyridin-2-amine

To a solution of dichloromethane (15 mL) was added 29(ii) (6-aminopyridin-2-yl)methanol (0.72 g, 5.8 mmol), tert-butyl(chloro)dimethylsilane (1.05 g, 6.95 mmol) and triethylamine (1.05 mL, 7.53 mmol). The mixture was stirred for 24 h and washed with saturated sodium bicarbonate (2×30 mL) and aqueous hydrochloric acid (2×30 mL, 0.1N). The organic layer was dried over anhydrous Na₂SO₄, filtered, and concentrated in vacuo. Purification was done using silica gel chromatography eluting with hexane:ethyl acetate (1:1) and fractions were combined and concentrated to afford the title product 29(iii) as a white solid (1.06 g, 70%). HPLC: Rₜ 2.58 min. (96.5% area). ¹H NMR (400 MHz, CDCl₃), δ: 7.34 (t, J=7.6 Hz, 1 H), 6.75 (d, J=7.6 Hz, 1 H), 6.25 (d, J=8.1 Hz, 1 H), 4.54 (s, 2 H), 4.27 (bs, 2 H), 0.84 (s, 9 H), 0.11 (s, 6 H); LRMS (ESI): m/z: 239.2.

### Intermediate 29(iv): N-[6-({[tert-Butyl(dimethyl)silyl]oxy}methyl)pyridin-2-yl]-2-oxo-1,3-oxazolidine-3-sulfonamide

To dichloromethane (5 mL) cooled to 0° C. was added the reagent 2-bromoethanol (0.27 mL, 3.3 mmol) and DIEA (1.23 mL, 7.5 mmol). Next, chlorosulfonyl isocyantae (0.23 g, 2.7 mmol) was added to the mixture over 0.25 h followed by stirring for 1 h. Intermediate 29(iii) (6-({[tert-butyl(dimethyl)silyl]oxy}-methyl)pyridin-2-amine) (0.58 g, 2.4 mmol) was dissolved in dichloromethane (10 mL) and then added to reaction mixture and stirred for 1 h. The work up was done by diluting the final reaction mixture with dichloromethane (30 mL) and washing with aqueous HCl (0.1 N, 2×20 mL) and saturated sodium bicarbonate (2×50 mL). The organic layer was dried over sodium sulfate and concentrated. The residue was placed under high vacuum for 24 h to afford the title compound 29(iv) as an oil (0.041 g, 62%). ¹H NMR (400 MHz, CDCl₃) δ ppm: 0.19 (s, 6 H), 0.97 (s, 9 H), 4.13-4.17 (m, 2 H), 4.39 (t, J=7.83 Hz, 2 H), 4.80 (s, 2 H), 6.60 (d, J=7.07 Hz, 1 H), 6.84 (d, J=9.09 Hz, 1 H), 7.66 (dd, J=8.84, 7.33 Hz, 1 H); LRMS (ESI): m/z (M+H): 388.2.

### Intermediate 29(v): N-[6-({[tert-Butyl(dimethyl)silyl]oxy}methyl)pyridin-2-yl]-4-(5-cyanopyridin-2-yl)piperazine-1-sulfonamide

The intermediate 29(iv) (N-[6-({(tert-butyl(dimethyl)silyl]oxy}methyl)pyridin-2-yl]-2-oxo-1,3-oxazolidine-3-sulfonamide) (0.17 g, 0.43 mmol), DIEA (0.18 mL, 1.1 mmol) and 6-piperazin-1-ylnicotinonitrile (0.093 g 0.50 mmol) were dissolved in acetonitrile (2 mL) in 5 mL microwave vessel and heated to 120° C. with microwave heat for 0.5 h, cooled to 25° C. Diluted reaction mixture with ethyl acetate (40 mL) and partitioned between aqueous HCl (0.5 N, 2×30 mL) and saturated sodium bicarbonate (2×30 mL). Dried organic layer over sodium sulfate and concentrated. The residue was purified by flash column chromatography through silica gel eluting with hexanes:ethyl acetate (3:1). The purified fractions were concentrated afford title product 29(v) as white foam (0.096 g, 46%). HPLC Rₜ: 3.881 min. (91.3%). ¹H NMR (400 MHz, CDCl₃) δ ppm: 0.13-0.20 (m, 6 H), 0.97 (s, 9 H), 3.32 (t, J=5.01 Hz, 4 H), 3.78 (t, J=4.99 Hz, 4 H), 4.70 (s, 2 H), 6.61 (d, J=8.84 Hz, 2 H), 6.87 (s, 1 H), 7.55 (dd, J=8.59, 7.07 Hz, 1 H), 7.64 (dd, J=9.09, 2.27 Hz, 1 H), 8.41 (d, J=2.02 Hz, 1 H); LRMS (ESI): m/z (M+H): 489.0.

### Example 29

### 4-(5-cyanopyridin-2-yl)-N-[6-(hydroxymethyl)pyridin-2-yl]piperazine-1-sulfonamide

The intermediate 29(v) (N-[6-({[tert-butyl(dimethyl)silyl]oxy}methyl)pyridin-2-yl]-4-(5-cyanopyridin-2-yl)piperazine-1-sulfonamide) was dissolved in ethanol (3 mL) and aqueous HCl (1 N, 3 mL) stirred for 2 h. Neutralized reaction mixture with triethyl amine (3 mL) and concentrated. The residue was by flash column chromatography through silica gel eluting with hexanes:ethyl acetate (3:1). The purified fractions were concentrated afford title product 29 as white solid (0.025, 35%). HPLC Rₜ: 2.148 (98.6%); LRMS (ESI): m/z (M+H): 375.1. See Table 1 below for ¹H NMR.

### Example 31

### N-(6-amino-4-methylpyridin-2-yl)-4-(5-fluoropyrimidin-2-yl)piperazine-1-sulfonamide

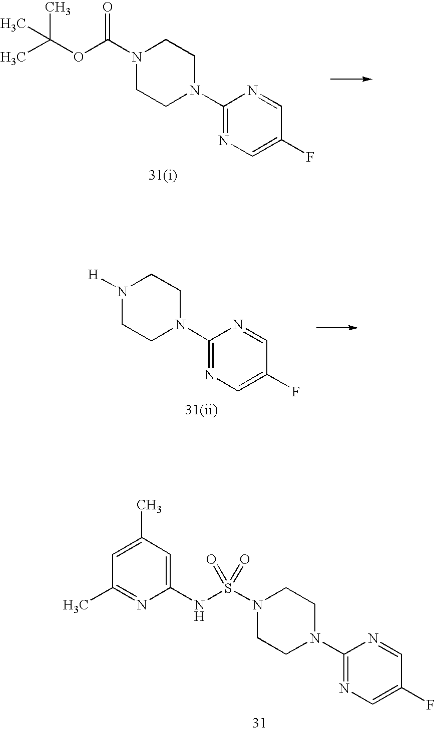

### Intermediate 31(i): Tert-butyl 4-(5-fluoropyrimidin-2-yl)piperazine-1-carboxylate

The title compound 31(i) was made by dissolving tert-butyl piperazine-1-carboxylate (0.53 g, 2.9 mmol) and 2-chloro-5-fluoropyrimidine (0.42 g, 3.2 mmol) in propan-2-ol (3 mL) and DIEA (1.1 mL, 6.4 mmol). The mixture was sealed under nitrogen in a microwave vessel, heated to 120° C. for 0.5 h with microwave energy and then cooled to 25° C. The solution was diluted with ethyl acetate (50 mL) and washed with 0.1 N HCl and saturated aqueous sodium bicarbonate (2×50 mL). The organic layer was dried over Na₂SO₄, then filtered through silica gel plug (5 mL) and concentrated to afford title product as a white solid (0.85 g, 80%). HPLC Rₜ: 3.485 min. (98.5%); ¹H NMR (400 MHz, CDCl₃), δ ppm: 1.49 (s, 9 H), 3.32-3.61 (m, 4 H), 3.64-3.95 (m, 4 H), 8.21 (s, 2 H); LRMS (ESI): m/z (M+H−t-Butyl): 227.1.

### Intermediate 351(ii): 5-fluoro-2-piperazin-1-ylpyrimidine hydrochloride

Intermediate 31(i) (tert-butyl 4-(5-fluoropyrimidin-2-yl)piperazine-1-carboxylate) (0.64 g, 2.3 mmol) was dissolved in 2N HCl, warmed to 50° C. and stirred for 3 h. The mixture was concentrated via in vacuo and placed under high vacuum for 24 h then triturated with diethyl ether (2×5 mL) to afford title compound 31(ii) as a white solid (0.48 g, 96%). HPLC Rₜ: 1.238 min. (99.5%); ¹H NMR (400 MHz, D₂O) δ ppm: 3.27 (t, J=5.3 Hz, 4 H), 3.91 (t, J=5.3 Hz, 4 H), 8.32 (s, 2 H); LRMS (ESI): m/z (M+H): 183.2.

### Example 31

### N-(6-amino-4-methylpyridin-2-yl)-4-(5-fluoropyrimidin-2-yl)piperazine-1-sulfonamide

The title compound was made using a method analogous to that described for Example 20, using instead intermediates 20(iii) (2,2-dimethyl-N-(4-methyl-6-{[(2-oxo-1,3-oxazolidin-3-yl)sulfonyl]amino}pyridin-2-yl)propanamide) and 31(ii) (5-fluoro-2-piperazin-1-ylpyrimidine hydrochloride). See Table 1 below for ¹H NMR.

### Example 32

### 4-(5-cyanopyridin-2-yl)-N-(6-isopropylpyridin-2-yl)piper-azine-1-sulfonamide

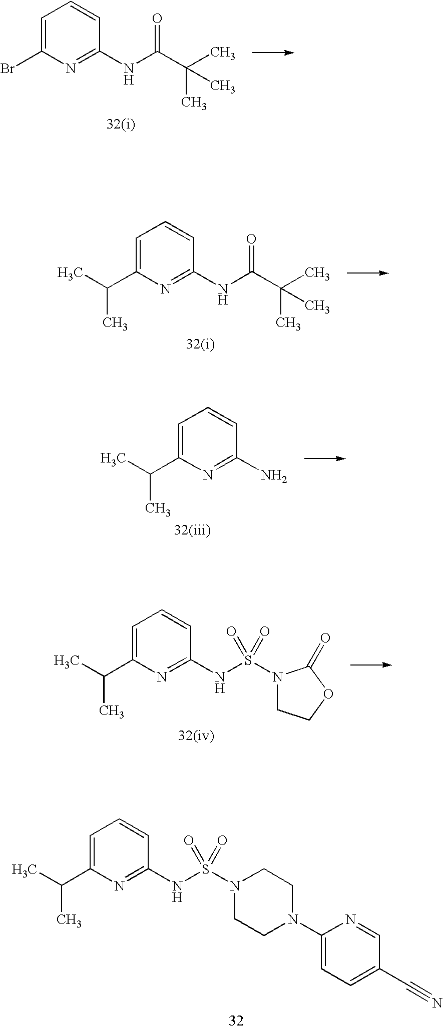

### Intermediate 32(i): N-(6-bromopyridin-2-yl)-2,2-dimethylpropanamide

The starting material 6-bromopyridin-2-amine (7.0 g, 40.5 mmol) was dissolved in 60 mL of CH₂Cl₂ and cooled to 0° C. using an ice bath. To the mixture was added 2,2-dimethylpropanoyl chloride (5.23 mL, 42.48 mL) dropwise followed by DIEA (13.6 mL, 82.9 mmol) addition. The solution was stirred for 1 h then diluted with 50 mL of diethyl ether. The mixture was partitioned between saturated aqueous sodium bicarbonate (2×50 mL) and the organic layer was dried over Na₂SO₄ and concentrated in vacuo. The residue was dissolved in ethyl acetate (10 mL) and hexane (20 mL) and allowed to stand for 3 hours to crystallize product. The product was filtered and rinsed with hexane/ethyl acetate (1:1) and dried under high vacuum to afford the title product 32(i) as white solid (9.56 g, 93%). HPLC Rt: 3.750 (97.8%). ¹H NMR (400 MHz, CD₃CN), δ: 8.22 (d, J=8.4 Hz, 1 H), 7.99 (bs, 1 H), 7.55 (t, J=8.1 Hz, 1 H), 7.22 (d, J=7.3 Hz, 1 H), 1.31 (s, 9 H); LCMS (ESI): m/z: 258.0.

### Intermediate 32(ii): N-(6-isopropylpyridin-2-yl)-2,2-dimethylpropanamide

The intermediate 32(i) (N-(6-bromopyridin-2-yl)-2,2-dimethylpropanamide) (5.0 g, 19.4 mmol) was dissolved in 100 mL of THF cooled to −78° C. using a dry ice bath. To the mixture was added CuI (7.40 g, 38.8 mmol) and the solution was stirred for 0.5 hours. Isopropyl magnesium chloride in THF (48.5 mL, 98 mmol, 2M) was then added dropwise at −78° C. and stirred at 25° C. for 2 hours. The reaction was quenched with saturated aqueous ammonium chloride, then 100 mL of ethyl acetate was added and the solids were removed by filtration. The solution was washed with saturated aqueous ammonium chloride (2×50 mL) and saturated aqueous sodium bicarbonate (2×50 mL). The organic layer was dried over Na₂SO₄ and concentrated to give amber oil. Purification was done using silica gel chromatography eluting with hexane/ethyl acetate (2:1). The purified fractions were collected and concentrated to afford the title product 32(ii) as amber oil (2.60 g, 60%). HPLC Rₜ: 3.050 (90.2%). ¹H NMR (400 MHz, CD₃CN), δ: 8.04 (d, J=7.8 Hz, 1 H), 7.97 (bs, 1 H), 7.63 (t, J=7.8 Hz, 1 H), 6.90 (d, J=7.5 Hz, 1 H), 2.95-2.88 (m, 1 H), 1.34 (s, 9 H), 1.28 (d, J=7.1 Hz, 6 H); LCMS (ESI): m/z: 221.2.

### Intermediate 32(iii): 6-isopropylpyridin-2-amine

To a solution of dioxane (5 mL) was added 32(ii) (N-(6-isopropylpyridin-2-yl)-2,2-dimethylpropanlamide) (2.0 g, 9.08 mmol) and HCl (9N, 10 mL). The mixture was stirred for 18 hours at 80° C., and then cooled to 25° C. The pH of the reaction mixture was adjusted to pH 9 using sodium hydroxide. The solution was diluted with 120 mL of ethyl acetate and washed with saturated aqueous sodium bicarbonate (2×30 mL). Next, the solution was azeotroped with toluene (10 mL) to afford 32(iii) (6-isopropylpyridin-2-amine) as clear oil (0.68 g, 55%). HPLC Rt: 2.017 (95.6%). ¹H NMR (400 MHz, CD₃CN), δ: 7.36 (t, J=7.8 Hz, 1 H), 6.64 (d, J=8.7, 1 H), 6.32 (d, J=8.1 Hz, 1 H), 2.92 (m, 1 H), 1.25 (d, J=4.5 Hz, 9 H); LCMS (ES): m/z: 137.2.

### Intermediate 32(iv): N-(6-isopropylpyridin-2-yl)-2-oxo-1,3-oxazolidine-3-sulfonamide

The title compound was made using a method analogous to that described for intermediate 1(i) above, using instead 6-isopropylpyridin-2-amine. After workup and concentration, the residue was triturated with diethyl ether, filtered and concentrated under high vacuum for 24 h to afford the title product 32(iv) as a white foam (0.84 g, 72%). ¹H NMR (400 MHz, CDCl₃) δ ppm: 1.32-1.41 (m, 6 H), 3.60-3.66 (m, 2 H), 3.70-3.80 (m, 1 H), 4.27-4.37 (m, 2 H), 6.61 (d, J=7.3 Hz, 1 H), 6.89 (d, J=8.6 Hz, 1 H), 7.67 (dd, J=8.6, 7.3 Hz, 1 H).

### Example 32

### 4-(5-cyanopyridin-2-yl)-N-(6-isopropylpyridin-2-yl)piper-azine-1-sulfonamide

The title compound was made using a method analogous to that described for Example 1 above, except using instead N-(6-isopropylpyridin-2-yl)-2-oxo-1,3-oxazolidine-3-sulfonamide and 6-piperazin-1-ylnicotinonitrile. After extraction and concentration, the residue was purified with flash chromatography using silica gel eluting with hexane/ethyl acetate (2:1), the purified fractions were collected, concentrated and dried on high vacuum for 2 hours to afford title product 32 as a white solid (0.86 g, 42%). HPLC Rt: 3.506 (97.2%). HRMS (ESI): Calcd for C₁₈H₂₃N₆O₂S m/z: 387.1603; Found: 387.1605; Anal. Calcd for C₁₈H₂₃N₆O₂S*(0.7 H₂O): C, 54.17; H, 5.91; N, 21.06; Found: C, 54.20; H, 5.81; N, 21.01. See Table 1 below for ¹H NMR.

### Example 33

### 4-(5-cyanopyridin-2-yl)-N-(6-methylpyridin-2-yl)piperidine-1-sulfonamide

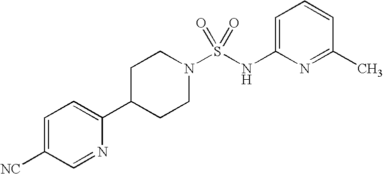

### Intermediate 33(i): tert-Butyl 4-(5-cyanopyridin-2-yl)piperidine-1-carboxylate

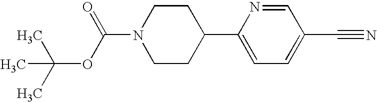

The starting reagent tert-butyl 4-iodopiperidine-1-carboxylate (2.0 g, 6.3 mmol), as made according literature procedure (Billotte, S; Syn. Lett., 4, 1998; 379-380), was dissolved in anhydrous THF (3 mL) under nitrogen atmosphere. Rieke zinc (1.0 N, 8.4 mL, 6.4 mmol) was added to the reaction mixture and it was then warmed to 65° C. and stirred for 0.5 h. 6-Bromonicotinonitrile (1.0 g, 5.7 mmol), DIEA (1.0 mL, 6.3 mmol), dppf (0.14 g, 0.16 mmol) and copper iodide (0.064 g, 0.32) were added to the reaction mixture and stirred for 18 h, After cooling to 25° C., the mixture was filtered through Celite®, diluted with ethyl acetate (50 mL) and washed with saturated sodium bicarbonate (2×30 mL). The organic layer was dried over sodium sulfate and then purified by flash column chromatography through silica gel eluting with hexanes: ethyl acetate (1:1). The purified fractions were concentrated to afford title product as white solid (1.6 g, 96%). HPLC Rₜ: 3.914 (86.4%); ¹H NMR (400 MHz, CDCl₃) δ ppm: 1.46-1.48 (m, 9 H), 1.69-1.75 (m, 2 H), 1.90 (d, J=12.88 Hz, 2 H), 2.84 (s, 1 H), 2.86-2.96 (m, 2 H), 4.27 (br. s., 2 H), 7.29 (d, J=8.08 Hz, 1 H), 7.90 (dd, J=8.21, 2.15 Hz, 1 H), 8.81 (d, J=1.77 Hz, 1 H).

### Intermediate 33(ii): 6-piperidin-4-ylnicotinonitrile

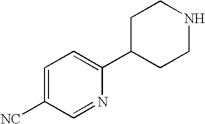

The intermediate 33(i) (tert-butyl 4-(5-cyanopyridin-2-yl)piperidine-1-carboxylate) (1.2 g, 4.4 mmol) was dissolved in dioxane followed by addition of HCl (aqueous 4N, 5.5 mL, 22 mmol) and stirred for 3 h, and then concentrated. The residue was triturated with diethyl ether (2×5 mL) to afford title product 33(ii) as white solid (0.82 g, 82%). HPLC Rₜ: 1.873 (97.4%); ¹H NMR (400 MHz, CD₃CN) δ ppm: 2.01-2.11 (m, 4 H), 3.02-3.14 (m, 3 H), 3.48 (d, J=12.88 Hz, 2 H), 7.43 (d, J=7.83 Hz, 1 H), 8.05 (dd, J=8.34, 2.27 Hz, 1 H), 8.48-8.59 (m, 1 H), 8.84 (d, J=1.52 Hz, 1 H); LRMS (ESI): m/z (M+H): 188.2.

### Example 33

### 4-(5-cyanopyridin-2-yl)-N-(6-methylpyridin-2-yl)piperidine-1-sulfonamide

The title compound was made using a method analogous to that described in Example 1 above, except using instead N-(6-methylpyridin-2-yl)-2-oxo-1,3-oxazolidine-3-sulfonamide and 6-piperidin-4-ylnicotinonitrile. After extraction and concentration, the residue was purified with flash chromatography using silica gel eluting with hexane/ethyl acetate (1:2) and the purified fraction were collected and concentrated. The residue was dissolved in methylene chloride (1 mL) and diethyl ether (5 mL) and HCl (1N in Et₂O) were added dropwise to form a precipitate. After filtration, the precipitate was placed under high vacuum for 2 hours to afford title product 33 as a white solid (0.026 g, 16%). HPLC Rt: 3.003 (96.1%). HRMS (ESI): Calcd for C₁₇H₂₀N₅O₂S m/z: 358.1338; Found: 358.1323; Anal. Calcd for C₁₇H₁₉N₅O₂S *(1.6 HCl): C, 49.11; H, 5.00; N, 16.84; Found: C, 49.09; H, 5.12; N, 16.60. See Table 1 below for ¹H NMR.

### Example 35

### N-1,5-naphthyridin-2-yl-4-[5-(trifluoromethyl)pyridin-2-yl]piperazine-1-sulfonamide

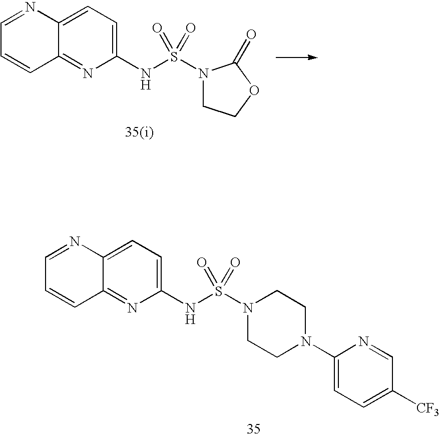

### Intermediate 35(i): N-1,5-naphthyridin-2-yl-2-oxo-1,3-xazoidine-3-sulfonamide

The title compound was made using a method analogous to that described for intermediate 1(i) above, except using 1,5-naphthyridin-2-amine. After workup and concentration, the residue was purified by flash column chromatography through silica gel eluting with hexanes:ethyl acetate (2:1). The purified fractions were concentrated afford title product as white solid (0.36 g, 29%). HPLC Rₜ: 2.158 (98.2%); LRMS (ESI) m/z (M+H): 295.1

### Example 35

### N-1,5-naphthyridin-2-yl-4-[5-(trifluoromethyl)pyridin-2-yl]piperazine-1-sulfonamide

The title compound was made using a method analogous to that described for Example 1 above, except using instead 35(i) (N-1,5-naphthyridin-2-yl-2-oxo-1,3-oxazolidine-3-sulfonamide) and 1-[5-(trifluoromethyl)pyridin-2-yl]piper-azine. See Table 1 below for ¹H NMR.

### Example 36

### N-1,5-naphthyridin-2-yl-4-[5-(trifluoromethyl)pyridin-2-yl]piperidine-1-sulfonamide

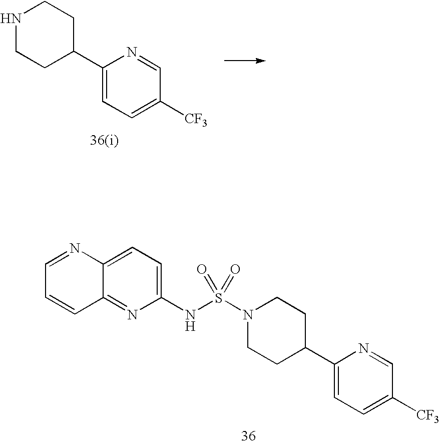

### Intermediate 36(i): 2-piperidin-4-yl-5-(trifluoromethyl)pyridine

The title compound was made using a method analogous to that described for intermediate 33(ii) above, except using tert-butyl 4-[5-(trifluoromethyl)pyridin-2-yl]piperidine-1-carboxylate (1.6 g, 4.8 mmol) as starting material. The residue was azeotroped with toluene and placed under high vacuum for 2 h to afford title compound as an amber solid (0.85 g, 77%). HPLC Rₜ: 2.417 (98.6%); ¹H NMR (400 MHz, CDCl₃) δ ppm: 2.20 (d, J=3.28 Hz, 4 H), 3.03-3.15 (m, 3 H), 3.58 (d, J=12.63 Hz, 2 H), 7.35 (d, J=8.34 Hz, 1 H), 7.92 (t, 1 H), 8.82 (s, 1 H); LRMS (ESI) m/z (M+H): 231.2.

### Example 36

### N-1,5-naphthyridin-2-yl-4-[5-(trifluoromethyl)pyridin-2-yl]piperidine-1-sulfonamide

The title compound was made using a method analogous to that described for Example 1 above, except using instead 35(i) (N-1,5-naphthyridin-2-yl-2-oxo-1,3-oxazolidine-3-sulfonamide) and 36(i) (2-piperidin-4-yl-5-(trifluoromethyl)-pyridine). See Table 1 below for ¹H NMR.

### Example 37

### 4-(5-chloro-3-methylpyridin-2-yl)-N-1,5-naphthyridin-2-ylpiperazine-1-sulfonamide

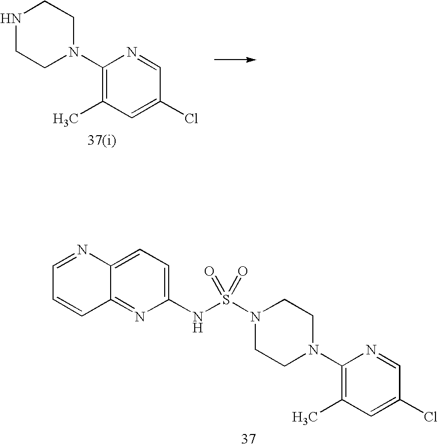

### Intermediate 37(i): 1-(5-chloro-3-methylpyridin-2-yl)piperazine

The title compound was made using a method analogous to that described for intermediate 23(i) above, except using starting reagent N-BOC-piperazine and 2-bromo-5-chloro-3-methylpyridine. After workup and concentration, title product 37(i) was obtained as an amber solid (0.10 g, 50%). HPLC Rₜ: 2.420 (98.4%); 1H NMR (400 MHz, CDCl₃) δ ppm: 2.27 (s, 3 H), 3.05-3.09 (m, 4 H), 3.12 (s, 1 H), 3.12 (d, J=3.54 Hz, 4 H), 7.40 (s, 1 H), 8.10 (s, 1 H); LRMS (ESI) m/z (M+H): 212.1.

### Example 37

### 4-(5-chloro-3-methylpyridin-2-yl)-N-1,5-naphthyridin-2-ylpiperazine-1-sulfonamide

The title compound was made using a method analogous to that described for Example 1 above, except using instead 35(i) (N-1,5-naphthyridin-2-yl-2-oxo-1,3-oxazolidine-3-sulfonamide and 37(i) (1-(5-chloro-3-methylpyridin-2-yl)piperazine). See Table 1 below for ¹H NMR.

### Example 40

### (3R)-4-(5-fluoropyrimidin-2-yl)-N-[6-(hydroxymethyl)pyridin-2-yl]-3-methylpiperazine-1-sulfonamide

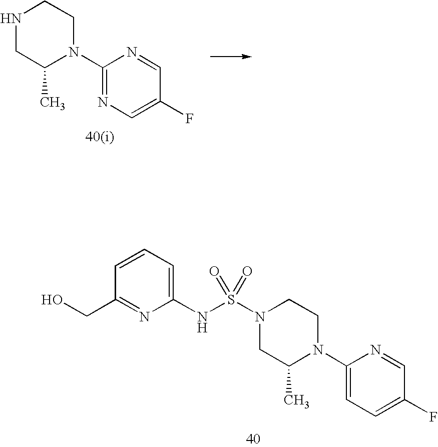

### Intermediate 40(i): 5-fluoro-2-[(2R)-2-methylpiperazin-1-yl]pyrimidine

The title compound was made using a method analogous to that described for intermediate 31(ii) above, except using instead tert-butyl (3R)-4-(5-fluoropyrimidin-2-yl)-3-methylpiperazine-1-carboxylate as starting material. LRMS (ESI): m/z (M+H): 197.2

### Example 40

### (3R)-4-(5-fluoropyrimidin-2-yl)-N-[6-(hydroxymethyl)pyridin-2-yl]-3-methylpiperazine-1-sulfonamide

The title compound was made using a method analogous to that described for Example 29 above, except using instead 40(i) (5-fluoro-2-[(2R)-2-methylpiperazin-1-yl]pyrimidine). See Table 1 below for ¹H NMR.

### Example 57

### N-[6-(2,5-dimethyl-1H-pyrrol-1-yl)pyridin-2-yl]-4-(4-fluorophenyl)piperidine-1-sulfonamide

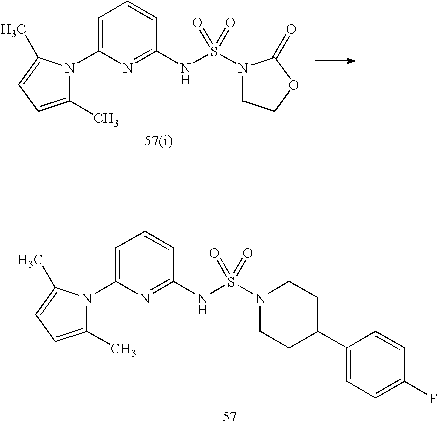

### Intermediate 57(i): N-[6-(2,5-dimethyl-1H-pyrrol-1-yl)pyridin-2-yl]-2-oxo-1,3-oxazolidine-3-sulfonamide

The title compound was made using a method analogous to that described for intermediate 1(i) above, except using instead, 6-(2,5-dimethyl-1H-pyrrol-1-yl)pyridin-2-amine (Org. Process Res. Dev. 2004, 8, 587).

### Example 57

### N-[6-(2,5-dimethyl-1H-pyrrol-1-yl)pyridin-2-yl]-4-(4-fluorophenyl)piperidine-1-sulfonamide

The title compound was made using a method analogous to that described for Example 1 above, except using instead 57(i) (N-[6-(2,5-dimethyl-1H-pyrrol-1-yl)pyridin-2-yl]-2-oxo-1,3-oxazolidine-3-sulfonamide) and 4-(4-fluorophenyl)piperidine. See Table 1 below for ¹H NMR.

### Example 58

### (3S)-4-(5-cyanopyridin-2-yl)-N-[6-(hydroxymethyl)pyridin-2-yl]-3-methylpiperazine-1-sulfonamide

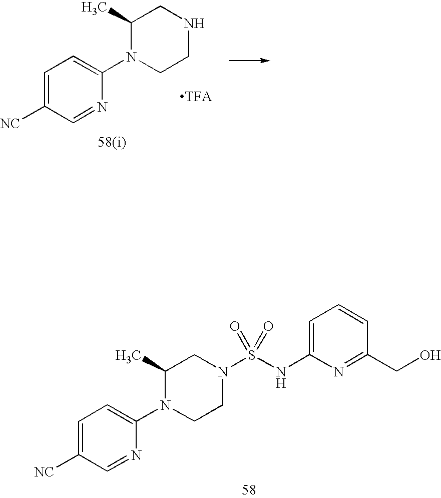

### Intermediate 58(i): 6-[(2S)-2-methylpiperazin-1-yl]nicotinonitrile trifluoroacetate

The title compound was made using a method analogous to that described for intermediate 23(i) above, except using instead tert-butyl (3S)-3-methylpiperazine-1-carboxylate as starting material. ¹H NMR (400 MHz, DMSO-d₆) δ ppm 9.29 (br. s., 1 H) 8.88 (br. s., 1 H) 8.56 (d, J=2.01 Hz, 1 H) 7.95 (dd, J=9.06, 2.27 Hz, 1 H) 6.98 (d, J=9.32 Hz, 1 H) 4.80-4.90 (m, 1 H) 4.39-4.48 (m, 1 H) 3.15-3.40 (m, 4 H) 2.96-3.08 (m, 1 H) 1.24 (d, J=6.80 Hz, 3 H).

### Example 58

### (3S)-4-(5-cyanopyridin-2-yl)-N-[6-(hydroxymethyl)pyridin-2-yl]-3-methylpiperazine-1-sulfonamide

The title compound was made using a method analogous to that described for Example 29 above, using instead 6-[(2S)-2-methylpiperazin-1-yl]nicotinonitrile trifluoroacetate as starting material. See Table 1 below for ¹H NMR.

### Example 59

### 4-(5-cyanopyridin-2-yl)-N-[6-(hydroxymethyl)pyridin-2-yl]-2-methyipiperazine-1-sulfonamide

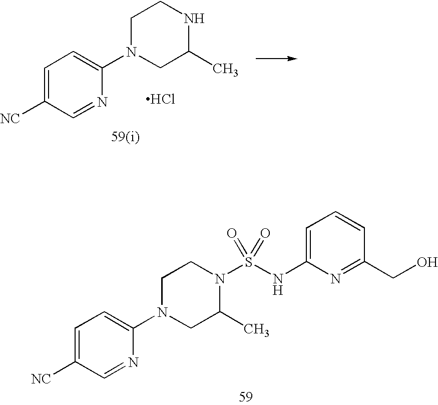

### Intermediate 59(i): 6-(3-methylpiperazin-1-yl)nicotinonitrile hydrochloride

The title compound was made using a method analogous to that described for intermediate 23(i) above, except using instead tert-butyl 2-methylpiperazine-1-carboxylate as starting material. LRMS (ESI): m/z (M+H): 203.2

### Example 59

### 4-(5-cyanopyridin-2-yl)-N-[6-(hydroxymethyl)pyridin-2-yl]-2-methylpiperazine-1-sulfonamide

The title compound was made using a method analogous to that described for Example 29 above, using instead 59(i) (6-(3-methylpiperazin-1-yl)nicotinonitrile hydro-chloride) as starting material. See Table 1 below for ¹H NMR.

Method B

The following examples illustrate Method B by which compounds of formula (I) and (III) of the invention can be prepared. Additional examples prepared by Method B are shown in Table 1 below.

### Example 2

### 4-(5-cyanopyridin-2-yl)-N-(6-methylpyridin-2-yl)piperazine-1-sulfonamide

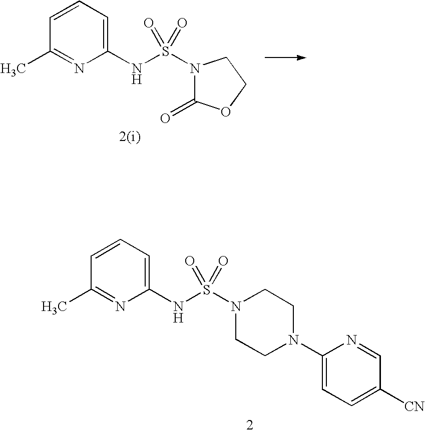

### Intermediate 2(i): N-(6-methylpyridin-2-yl)-2-oxo-1,3-oxazolidine-3-sulfonamide

Intermediate 2(i) was made using a method analogous to that described for intermediate 1(i) above, using instead 2-amino-6-picoline (9.0 g, 83 mmol), chlorosulfonyl isocyanate (23.0 mL, 83 mmol), 2-chloroethanol (5.5 mL, 4.1 mmol), triethylamine (35.0 mL, 249 mmol), and dichloromethane (270 mL). The product 2(i) was obtained as a white solid after trituration with hexanes. ¹H NMR (400 MHz, CDCl₃), δ: 12.34 (s, 1 H) 7.62 (dd, J=8.8, 7.3 Hz, 1 H) 6.77 (d, J=8.8 Hz, 1 H) 6.57 (d, J=7.1 Hz, 1 H) 4.39 (t, J=8.0 Hz, 2 H) 4.15 (t, J=7.8 Hz, 2 H) 2.50 (s, 3 H).

### Example 2

### 4-(5cyanopyridin-2-yl)-N-(6-methylpyridin-2-yl)piperazine-1-sulfonamide

6-Piperazinonicotinonitrile (0.34 g, 1.8 mmol, 2.0 equiv), intermediate 2(i) (N-(6-methylpyridin-2-yl)-2-oxo-1,3-oxazolidine-3-sulfonamide) (0.23 g, 0.9 mmol, 1 equiv), and triethylamine (1 mL) in acetonitrile (20 mL) were refluxed for 16 h. Cooled to room temperature and concentrated in vacuo. The residual solid was then purified by flash column chromatography on silica gel eluting with a gradient dichloromethane:acetonitrile:methanol (94:4:2) to dichloro-methane:methanol (90:10) to give a residual solid. Recrystallization from dichloromethane:hexanes provided a light cream colored solid 2 (0.054 g, 17%). See Table 1 below for ¹HNMR.

### Example 3

### 4-(5-cyanopyridin-2-yl)-N-(6-ethylpyridin-2-yl)piperazine-1-sulfonamide

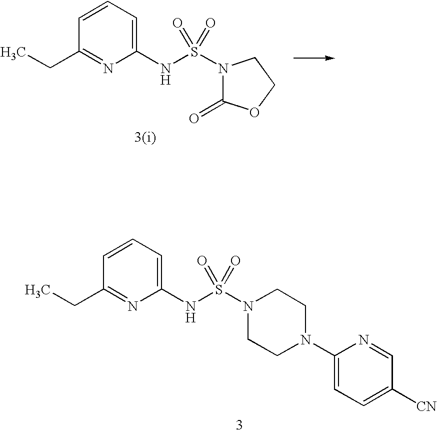

### Intermediate 3(i): N-(6-ethylpyridin-2-yl)-2-oxo-1,3-oxazolidine-3-sulfonamide

Intermediate 3(i) was made using a method analogous to that described for intermediate 1(i) above, using instead 2-amino-6-ethylpyridine (0.5 g, 4.1 mmol), chlorosulfonyl isocyanate (0.37 mL, 4.1 mmol), 2-chloroethanol (0.27 mL, 4.1 mmol), triethylamine (1.7 mL, 6.2 mmol), and dichloromethane (60 mL). The product 3(i) was obtained as a white solid after workup and drying (0.53 g, 48%). ¹H NMR (400 MHz, CDCl₃), δ: 12.36 (s, 1 H) 7.64 (dd, J=8.8, 7.3 Hz, 1 H) 6.76 (d, J=8.6 Hz, 1 H) 6.58 (d, J=7.3 Hz, 1 H) 4.39 (t, J=8.0 Hz, 2 H) 4.15 (t, J=7.8 Hz, 2 H) 2.78 (q, J=7.6 Hz, 2 H) 1.35 (t, J=7.6 Hz, 3 H).

### Example 3

### 4-(5cyanopyridin-2-yl)-N-(6-ethylpyridin-2-yl)piperazine-1-sulfonamide

The title compound was made using a method analogous to that described for Example 2 above, using instead intermediate 3(i) (N-(6-ethylpyridin-2-yl)-2-oxo-1,3-oxazolidine-3-sulfonamide) (0.2 g, 0.74 mmol, 1 equiv.) and 6-piperazinonicotinonitrile (0.4 g, 2.2 mmol, 3.0 equiv). See Table 1 below for ¹HNMR.

### Example 60

### N-(6-ethylpyridin-2-yl)-4-pyrimidin-2-ylpiperidine-1-sulfonamide

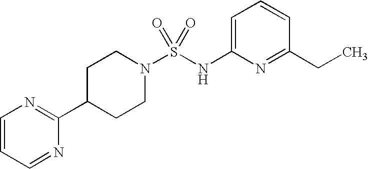

A mixture of 2-piperidin-4-ylpyrimidine (163 mg, 1.0 mmol, 2.0 eq.), intermediate 3(i) (N-(6-ethylpyridin-2-yl)-2-oxo-1,3-oxazolidine-3-sulfonamide) (135 mg, 0.5 mmol, 1.0 eq), and DIEA (0.26 mL, 1.5 mmol, 3 eq.) in 4 mL anhydrous DMF was heated at 100° C. for 16 h. After the reaction mixture was cooled to ambient temperature, the solvent and volatiles were removed in vacuo. The residues were subject to preparatory HPLC via a gradient elution of decreasing polarity (85%:15% water:acetonitrile→10%:90% water:acetonitrile in 0.1% TFA) to afford the desired product 60 (119.4 mg, 68%). See Table 1 below for ¹HNMR.

### Examples 61-69

Examples 61 to 69 were prepared using methods analogous to Example 60 above, except that the 2-piperidin-4-ylpyrimidine was replaced with the corresponding amine group. In particular, the 3,4-dihydrospirochromene-2,4′-piperidine intermediates of Examples 63 to 66 were prepared using methods described in WO 2000059510; WO 9528397; U.S. Pat. No. 5,760,054 and by Fletcher et al., J. Med. Chem. 2002, 45, 492-503.

Method C

The following examples illustrate Method C by which compounds of formula I of the invention can be prepared.

### Example 16

### N-[6-(2-hydroxyethyl)pyridin-2-yl]-1,2,4,5-tetrahydro-3H-3-benzazepine-3-sulfonamide

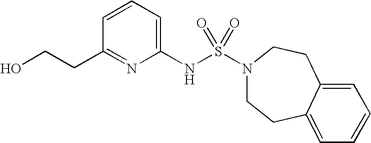

Lithium aluminum hydride (0.75 mL, 1M in THF) was added slowly to a flask containing a solution of ethyl {6-[(1,2,4,5-tetrahydro-3H-3-benzazepin-3-ylsulfonyl)amino]pyridin-2-yl}acetate (218 mg) in anhydrous THF (3 mL) at a temperature of about 0° C. At the end of the addition, the reaction mixture was allowed to warm to room temperature. After stirring ovemight, the reaction mixture was diluted with anhydrous CH₂Cl₂ (6 mL) and cooled to 0° C., then quenched with 0.05 mL of water, 0.05 mL of 1N NaOH, and 0.15 mL of water. After stirring for 20 min, the reaction mixture was filtered through a Celite® pad. The pad was washed with CH₂Cl₂ (2×50 mL), Et₂O (2×50 mL), 5% MeOH:EtOAc (50 mL), 10% MeOH:EtOAc (100 mL). All the organic washes were combined and concentrated under reduced pressure to give a residue. The crude mixture was purified by using silica gel chromatography eluting with MeOH:CH₂Cl₂ to give the title compound (85 mg, 44%). See Table 1 below for ¹HNMR.

Method D

The following examples illustrate Method D by which compounds of formula I of the invention can be prepared.

### Example 17

### N,N-diethyl-2-{6-[(1,2,4,5-tetrahydro-3H-3-benzazepin-3-ylsulfonyl)amino]pyridin-2-yl}acetamide

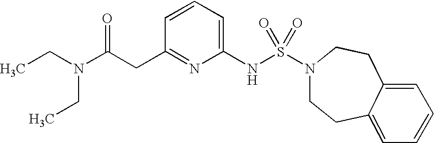

Dimethylaluminium chloride in 1M of hexane (1.2 mL) was added to a solution of diethylamine (0.12 mL) in anhydrous CH₂Cl₂ (5 mL) under nitrogen at about 0° C. After stirring at about 0° C. for 20 min, and at room temperature for 40 min, a solution of ethyl {6-[(1,2,4,5-tetrahydro-3H-3-benzazepin-3-ylsulfonyl)amino]pyridin-2-yl}acetate (120 mg) in anhydrous CH₂Cl₂ (2 mL) was added to the reaction mixture. After stirring at room temperature for 21 h, the reaction mixture was diluted with CH₂Cl₂ (50 mL) and quenched with water (10 mL) and 20% aqueous Rochelle's salt (10 mL) at about 0° C. The reaction mixture was diluted with CH₂Cl₂ (20 mL). The layers were separated and the organic layer was washed with brine, dried over K₂CO₃, filtered and concentrated under reduced pressure. The crude residue was purified by silica gel chromatography eluting with MeOH:CH₂Cl₂ to give the title compound (74 mg, 59%). See Table 1 below for ¹HNMR.

The following Table 1 depicts further Ki, % inhibition, structure, nomenclature, and NMR data of further embodiments of the Invention. Unless otherwise mentioned, compounds in Table 1 were synthesized starting from commercially available materials.

TABLE 1

| | | % | | | |
|---|---|---|---|---|---|
| | | Inh | | | |
| | Ki | @ | | | |
| | app | 0.1 | | | |
| Ex. # | (nM) | μM | Structure | Method | ¹H NMR |
| 1 | 4.1 | 95.9 | 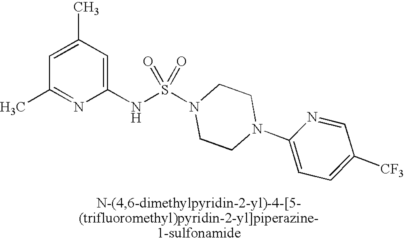 | A | ¹H NMR (400 MHz, CD₃CN), δ: 9.84 (bs, 1 H), 8.43 (s, 1 H), 7.74 (dd, J=11.4, 1.8 Hz, 1 H), 6.83-6.78 (m, 2 H), 6.46 (s, 1 H), 3.74 (t, J=4.6 Hz, 4 H), (3.23 (t, J=4.6 Hz, 4 H), 2.32 (s, 3 H), 2.26 (s, 1 H). |
| 2 | 17.4 | 100 | 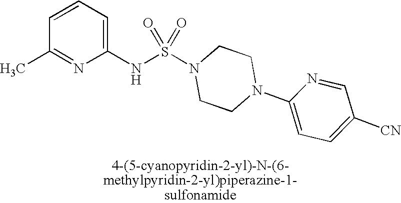 | B | ¹H NMR (400 MHz, CDCl₃), δ: 10.12 (s, 1 H) 8.39 (d, J=1.8 Hz, 1 H) 7.62 (dd, J=9.1, 2.3 Hz, 1 H) 7.48 (dd, J=8.7, 7.2 Hz, 1 H) 6.82 (d, J=8.6 Hz, 1 H) 6.60 (d, J=8.8 Hz, 1 H) 6.51 (d, J=7.1 Hz, 1 H) 3.76 (m, 4 H) 3.32 (m, 4 H) 2.40 (s, 3 H). |
| 3 | 9 | 88.5 | 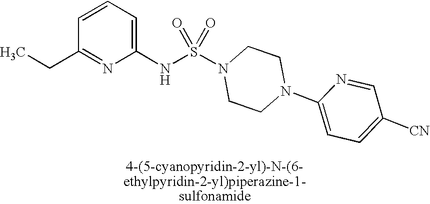 | B | ¹H NMR (400 MHz, CDCl₃), δ: 10.32 (s, 1 H) 8.39 (d, J =2.3 Hz, 1 H) 7.62 (dd, J =9.1, 2.3 Hz, 1 H) 7.50 (dd, J=8.8, 7.3 Hz, 1 H) 6.79 (d, J=8.6 Hz, 1 H) 6.60 (d, J=9.1 Hz, 1 H) 6.50 (d, J=7.3 Hz, 1 H) 3.74-3.80 (m, 4 H) 3.27-3.35 (m, 4 H) 2.67 (q, J=7.5 Hz, 2 H) 1.28 (t, J=7.6 Hz, 3 H). |
| 4 | NA | 26.6 | 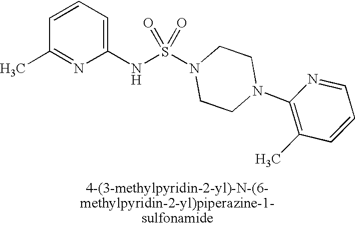 | A | ¹H NMR (400 MHz, CD₃OD), δ: 8.05 (d, J=7.3 Hz, 1 H), 8.00 (d, J=6.1 Hz, 1 H), 7.76 (t, J=7.9 Hz, 1 H), 7.25 (t, J=6.3 Hz, 2 H), 6.81 (d, J=7.6 Hz, 1 H), 3.47 (q, J=4.6 Hz, 2 H), 3.40 (q, J=5.5 Hz, 2 H), 2.40 (s, 3 H), 2.35 (s, 3 H). |
| 5 | NA | 44.6 | 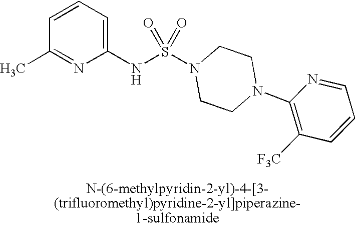 | A | ¹H NMR (400 MHz, CD₃CN), δ: 8.40 (d, J=3.8 Hz, 1 H), 8.06 (t, J=7.8 Hz, 1 H), 7.53 (d, J=8.8 Hz, 1 H), 7.15-7.12 (m, 2H), 3.42 (q, J=5.6 Hz, 4 H), 3.26 (q, J=5.1 Hz, 4 H), 2.55 (s, 3 H). |
| 6 | NA | 56.2 | 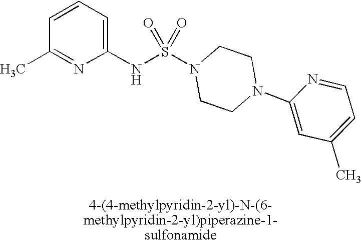 | A | ¹H NMR (400 MHz, CD₃CN), δ: 8.03 (t, J=7.8 Hz, 1 H), 7.94 (d, J=6.3 Hz, 1 H), 7.50 (d, J=8.9 Hz, 1 H), 7.10 (d, J=7.3 Hz, 1 H), 7.05 (s, 1 H), 6.88 (d, J=6.3 Hz, 1 H), 4.01 (q, J=5.1 Hz, 4 H), 3.54 (q, J=5.0 Hz, 4 H), 2.61 (s, 3 H), 2.47 (s, 4 H). |
| 7 | NA | 17.7 | 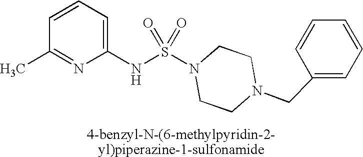 | A | ¹H NMR (400 MHz, CD₃CN), δ: 12.98 (bs, 1 H), 7.71 (t, J=8.6 Hz, 1 H), 7.63-7.61 (m, 2 H), 7.38-7.36 (m, 3 H), 7.08 (d, J=8.6 Hz, 1 H), 6.73 (d, J=7.3 Hz, 1 H), 4.14 (s, 2 H), 3.65 (bs, 2 H), 3.51 (bs, 2H), 3.20 (bs, 2 H), 2.98 (bs, 2 H), 2.40 (s, 3 H). |
| 8 | 2.1 | 96.4 | 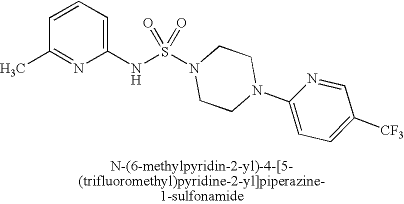 | A | ¹H NMR (400 MHz, CD₃CN), δ: 8.31 (s, 1 H), 8.17 (t, J=8.6 Hz, 1 H), 8.15 (d, J=9.1 Hz, 2 H), 7.65 (d, J=8.6 Hz, 1 H), 7.23 (dd, J=8.6 7.6, 3.4 Hz, 2 H), 4.04 (d, J=4.8 Hz, 4 H), 3.57 (d, J=4.8 Hz, 4 H), 2.64 (s, 3 H). |
| 9 | 14 | 86.5 | 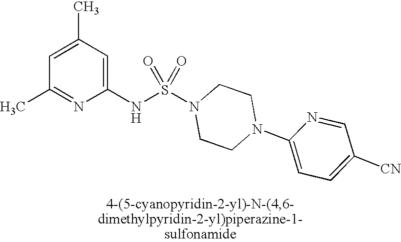 | A | ¹H NMR (400 MHz, CD₃CN), δ: 9.84 (bs, 1 H), 8.43 (s, 1 H), 7.74 (dd, J=11.4, 1.8 Hz, 1 H), 6.83-6.78 (m, 2 H), 6.46 (s, 1 H), 3.74 (t, J=4.6 Hz, 4 H), (3.23 (t, J=4.6 Hz, 4 H), 2.32 (s, 3 H), 2.26 (s, 1 H). |
| 10 | 25.5 | 79.9 | 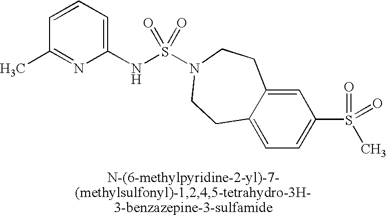 | B | ¹H NMR (400 MHz, CD₃OD) δ ppm 2.37 (s, 3 H) 3.03-3.12 (m, 7 H) 3.47 (s, 2 H) 3.49 (d, J=4.55 Hz, 2 H) 6.70 (d, J=7.33 Hz, 1 H) 6.99 (d, J=8.59 Hz, 1 H) 7.38 (d, J=7.83 Hz, 1 H) 7.57 (dd, J=8.59, 7.33 Hz, 1 H) 7.66-7.72 (m, 2 H). |
| 11 | 2.8 | 95.5 | 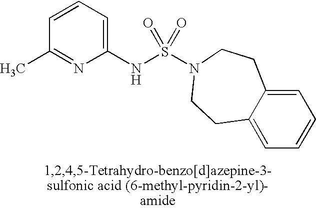 | B | ¹H NMR (400 MHz, CDCl₃) δ ppm 2.42 (3 H, s) 2.94-2.99 (4 H, m) 3.44-3.52 (4 H, m) 6.67 (1 H, d, J=7.58 Hz) 6.93(1 H, d, J=8.34 Hz) 7.06-7.16 (4 H, m) 7.46-7.54 (1 H, m). |
| 12 | 8.6 | 100 | 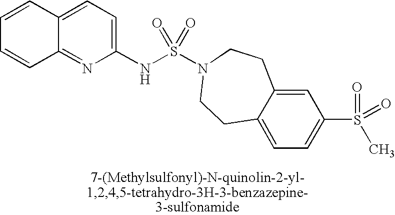 | A | ¹H NMR (400 MHz, CDCl₃), δ ppm 3.14 (s, 3 H) 3.22-3.31 (m, 4 H) 3.62 (s, 2 H) 3.63 (d, J=1.52 Hz, 2 H) 6.89 (d, J=9.09 Hz, 1 H) 7.40-7.47 (m, 2 H) 7.48 (s, 1 H) 7.71 (t, J=7.45 Hz, 2 H) 7.79 (s, 1 H) 7.82 (dd, J=7.71, 1.89 Hz, 1 H) 7.93 (d, J=9.35 Hz, 1 H). |
| 13 | 7.3 | 94.9 | 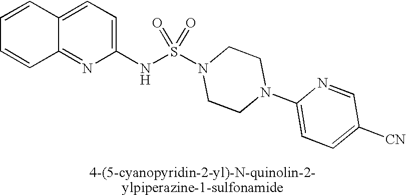 | A | ¹H NMR (400 MHz, DMSO-d₆), δ ppm 3.14 (m, 4 H) 3.78 (m, 4 H) 6.95 (d, J=9.09 Hz, 1 H) 7.35 (t, J=7.45 Hz, 1 H) 7.50 (d, J=7.83 Hz, 1H) 7.55 (d, J=7.83 Hz, 1 H) 7.60-7.67 (m, 1 H) 7.80 (d, J=7.83 Hz, 1 H) 7.86 (dd, J=9.09, 2.27 Hz, 1 H) 8.19 (d, J=9.60 Hz, 1 H) 8.48 (d, J=2.02 Hz, 1 H) 12.78 (s, 1 H) 12.78 (br, 1H). |
| 14 | 4.8 | 100 | 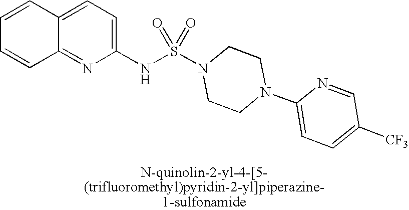 | B | ¹H NMR (400 MHz, DMSO-d₆), δ ppm 3.14 (d, J=5.05 Hz, 4 H) 3.65-3.74 (m, 4 H) 6.93 (d, J=9.09 Hz, 1 H) 7.30 (t, J=7.58 Hz, 1 H) 7.44 (br, 1 H) 7.50 (d, J=8.08 Hz, 1 H) 7.56-7.62 (m, 1 H) 7.73-7.78 (m, 2 H) 8.14 (d, J=9.60 Hz, 1 H) 8.36 (s, 1 H) 12.72 (s, 1 H). |
| 15 | 2 | 100 | 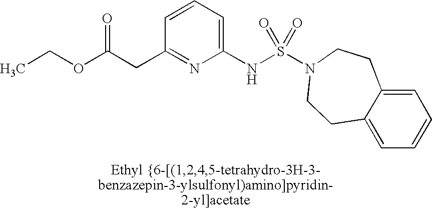 | A | ¹H NMR (400 MHz, CD₃OD) δ ppm 1.44-1.54 (m, 3 H) 3.27-3.38 (m, 4 H) 3.80-3.84 (m, 4 H) 4.02-4.12 (m, 2 H) 4.30-4.42 (m, 2 H) 7.26-7.37 (m, 2 H) 7.42-7.52 (m, 4 H) 7.99 (td, J=7.89, 2.40 Hz, 1 H) |
| 16 | 2.4 | 100 | 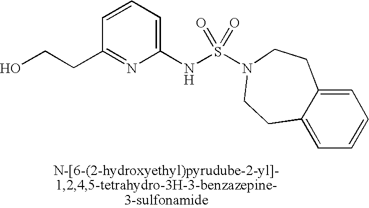 | C | ¹H NMR (400 MHz, CD₃OD), δ ppm 2.76 (2 H, t, J=6.32) 2.85 (4 H, m) 3.37 (4 H, m) 3.75 (2 H, t, J=6.32) 6.72 (1 H, d) 6.85 (1 H, d) 7 (4 H, s) 7.5 (1 H, m). |
| 17 | 5.7 | 100 | 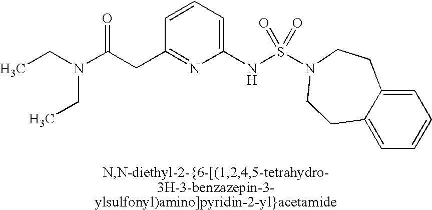 | D | ¹H NMR (400 MHz, CD₃OD), δ ppm 1.1 (6 H, t) 2.95 (4 H, m) 3.5 (4 H, m) 3.45 (4 H, m) 3.75 (1 H, s) 5.45 (1 H, s) 6.85 (1 H, d) 6.95 (1 H, d) 7.15 (4H, s) 7.65 (1 H, m). |
| 18 | 13 | 78.18 | 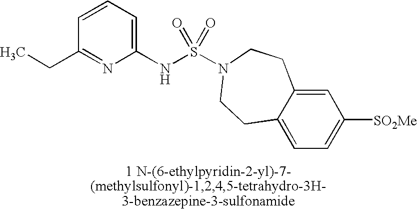 | A | ¹H NMR (400 MHz, CDCl₃) δ ppm 1.27 (t, J=7.58 Hz, 3 H) 2.68 (q, J=7.58 Hz, 2 H) 3.03 (s, 3 H) 3.07-3.12 (m, 4 H) 3.48-3.55 (m, 4 H) 6.62 (d, J=7.33 Hz, 1 H) 6.86 (d, J=8.59 Hz, 1 H) 7.29 (d, J=7.83 Hz, 1 H) 7.53 (dd, J=8.46, 7.45 Hz, 1 H) 7.66-7.68 (m, J=1.77 Hz, 1 H) 7.70 (dd, J=7.83, 1.77 Hz, 1H) |
| 19 | 2.2 | 96.9 | 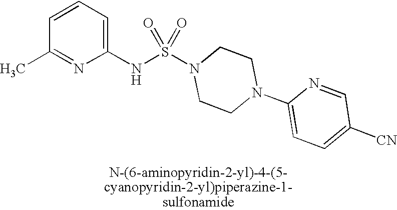 | A | ¹H NMR (400 MHz, CD₃OD) δ ppm 3.32-3.36 (m, 4 H) 3.76-3.81 (m, 4 H) 6.35 (d, J=8.34 Hz, 1 H) 6.44 (d, J=7.83 Hz, 1 H) 6.88 (d, J=9.09 Hz, 1 H 7.59 (t, J=8.21 Hz, 1 H) 7.75 (dd, J=9.09, 2.53 Hz, 1 H) 8.41 (d, J=2.27 Hz, 1 H) |
| 20 | 2.5 | 100 | 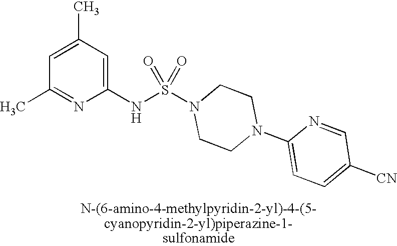 | A | ¹H NMR (400 MHz, DMSO-d₆) δ ppm 2.07 (s, 3 H) 3.04-3.21 (m, 4 H) 3.65-3.77 (m, 4 H) 5.75 (s, 1 H) 6.06 (s, 1 H) 6.93 (d, J=9.09 Hz, 1 H) 7.86 (dd, J=9.09, 2.27 Hz, 1 H) 8.48 (d, J=2.02 Hz, 1 H) |
| 21 | NA | 10.6 | 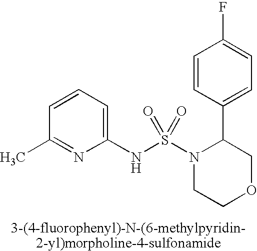 | A | ¹H NMR (400 MHz, CDCl₃), δ ppm: 10.02 (bs, 1 H), 7.49 (t, J=7.4 Hz, 1 H), 7.35-7.33 (m, 2 H), 7.03 (t, J=8.90 Hz, 2 H), 6.84 (d, J=8.60 Hz, 1 H), 6.52 (d, J=8.60 Hz, 1 H), 4.54 (dd, J=10.6, 2.6 Hz, 1 H), 4.08 (dd, J=11.6, 2.0 Hz, 1 H), 3.82 (td, J=11.7, 2.6 Hz, 1 H), |
| # 3.69 (dd, J=9.6, 2.0 Hz, 1 H), 3.60 (d, J=11.7 Hz, 1 H), 3.01 (td, J=11.9, 3.3 Hz, 1 H), 2.75 (t, J=11.1 Hz, 1 H), 2.40 (s, 3 H). | | | | | |
| 22 | 22 | 90 | 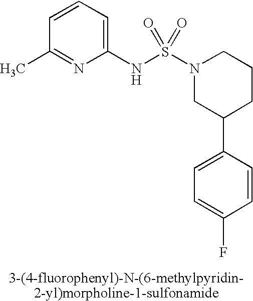 | A | ¹H NMR (400 MHz, CDCl₃), δ ppm: 9.02 (bs, 1 H), 7.49 (t, J=8.4 Hz, 1 H), 7.17-7.14 (m, 2 H), 6.98 (t, J=8.6 Hz, 2 H), 6.91 (d, J=8.60 Hz, 1 H), 6.61 (d, J=7.4 Hz, 1 H), 3.84 (d, J=11.4 Hz, 1 H), 2.83-2.70 (m, 3 H), 2.41 (s, 3 H), 1.97-1.93 (m, 1 H), 1.87-1.82 (m, 1 H), 1.74-1.69 (m, 1 H), 1.52-1.48 (m, 1 H). |
| 23 | 19.8 | 100 | 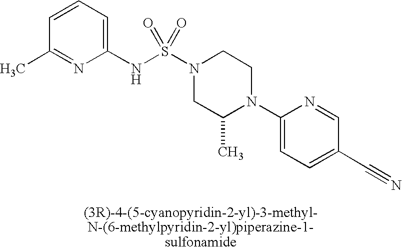 | A | ¹H NMR (400 MHz, CDCl₃) δ ppm: 1.26 (d, J=6.9 Hz, 3 H), 2.63 (s, 3 H), 3.20 (m, 1 H), 3.35 (m, 1 H), 3.45-3.49 (m, 2 H), 3.77 (m, 1 H), 4.26-4.33 (m, 2 H), 6.99 (d, J=7.6 Hz, 1 H), 7.26 (t, J=7.6 Hz, 1 H), 7.44 (d, J=7.3 Hz, 1 H), 7.83 (d, J=7.3 Hz, 1 H), 8.15 (m, 1 H), 8.41 (s, 1 H). |
| 24 | NA | 0 | 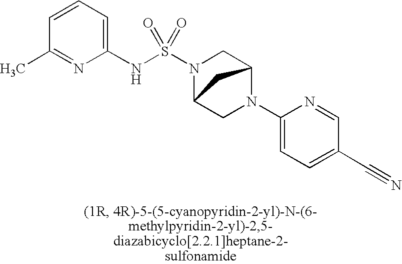 | A | ¹H NMR (400 MHz, CDCl₃) δ ppm: 1.26 (s, 1 H), 1.95 (d, J=9.6 Hz, 1 H), 2.12 (d, J=9.9 Hz, 1 H), 2.40 (s, 3 H), 3.39 (m, 1 H), 3.47 (t, J=8.0 Hz, 2 H), 3.74 (d, J=8.3 Hz, 1 H), 4.63 (s, 1 H), 6.31 (d, J=8.1 Hz, 1 H), 6.55 (d, J=7.1 Hz, 1 H), 6.79 (d, J=8.6 Hz, 1 H), 7.47 (t, J=8.0 Hz, 1 H), 7.59 (d, J=8.8 Hz, 1 H), 8.34 (s, 1 H), 10.40 (brs, 1 H). |
| 25 | 63.5 | 50 | 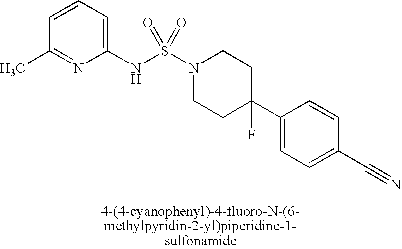 | A | ¹H NMR (400 MHz, CDCl₃) δ ppm: 1.94-2.04 (m, 2 H), 2.11 (m, 1 H), 2.21 (m, 1 H,) 2.46 (S, 3 H), 3.21 (t, J=11.6 Hz, 2 H), 3.84 (dd, J=8.1, 4.3, 2 H), 6.62 (d, J=7.1 Hz, 1 H), 7.00 (d, J=8.6 Hz, 1 H), 7.45 (d, J=8.2 Hz, 2 H), 7.53 (m, 1 H), 7.67 (d, J=8.1 Hz, 2 H). |
| 26 | 1.6 | 100 | 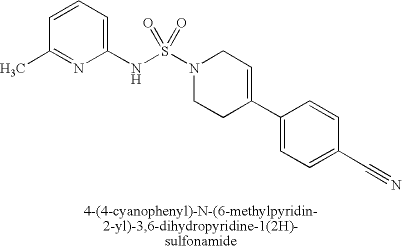 | A | ¹H NMR (400 MHz, CDCl₃) δ ppm: 2.44 (s, 3 H), 2.60 (s, 2 H), 3.53 (t, J=5.4 Hz, 2 H), 3.98-4.04 (m, 2 H), 6.17 (m, 1 H), 6.61 (d, J=7.1 Hz, 1 H), 6.98 (d, J=8.1 Hz, 1 H), 7.42-7.44 (m, 2 H), 7.52 (t, J=8.1 Hz, 1 H), 7.58-7.62 (m, 2 H). |
| 27 | 2.0 | 100 | 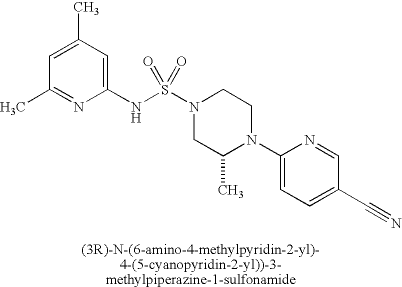 | A | ¹H NMR (400 MHz, CDCl₃), δ ppm: 8.41 (s, 1 H), 7.63 (dd, J=8.8, 2.0 Hz, 1 H), 6.69 (s, 1 H), 6.57 (d, J=9.1 Hz, 1 H), 5.86 (s, 1 H), 5.66 (bs, 2 H), 4.64 (bs, 1 H), 4.25 (d, J=12.4 Hz, 1 H), 3.79 (d, J=10.9 Hz, 1 H), 3.64 (d, J=11.6 Hz, 1 H), 3.20 (td, J=12.6, 3.0 Hz, 1 H), 3.01 (dd, J=11.7, 3.5 Hz, 1 H), |
| # 2.86 (td, J=11.6, 3.3 Hz, 1 H), 2.22 (s, 3 H), 1.20 (d, J=6.6, 3 H). | | | | | |
| 28 | 0.66 | 100 | 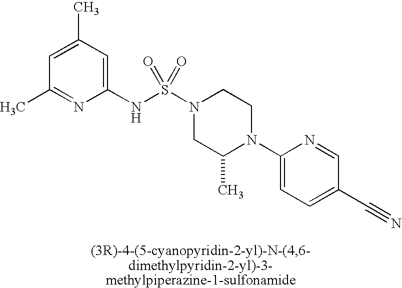 | B | ¹H NMR (400 MHz, CDCl₃) δ ppm: 1.29 (d, J=6.8 Hz, 3 H), 2.26 (s, 3 H), 2.38 (s, 3 H), 2.77-2.89 (m, 1 H), 2.97 (dd, J=11.6, 3.6 Hz, 1 H), 3.27 (td, J=12.9, 3.6 Hz, 1 H), 3.61 (dd, J=11.6, 1.8 Hz, 1 H) 3.74-3.85 (m, 1 H), 4.25-4.36 (m, 1 H). 4.63-4.74 (m, 1 H), 6.32 (s, 1 H), 6.58 (d, J=8.8 Hz, 1 H), |
| # 6.66 (s, 1 H), 7.63 (dd, J=8.8, 2.40 Hz, 1 H), 8.41 (d, J=2.3 Hz, 1 H). | | | | | |
| 29 | 13 | 95 | 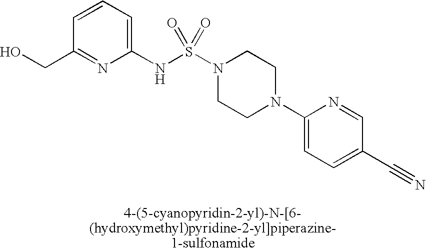 | A | ¹HNMR (400 MHz, CDCl₃), δ ppm: 8.40 (s, 1 H), 7.67-7.61 (m, 2 H), 7.06 (d, J=8.3 Hz, 1 H), 6.79 (d, J=7.3 Hz, 1 H), 6.61 (d, J=9.1 Hz, 1 H), 4.70 (s, 2 H), 3.81 (br s, 1 H), 3.75 (t, J=5.1 Hz, 1 H), 3.37 (t, J=5.2 Hz, 1 H). |
| 30 | >1 | 100 | 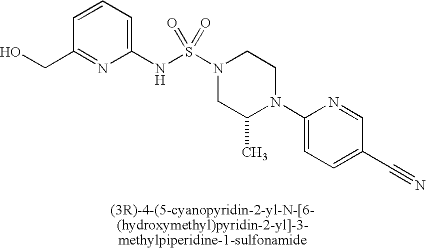 | A | ¹H NMR (400 MHz, CDCl₃-d) δ ppm: 1.21 (d, J=6.8 Hz, 3 H), 2.93 (m, 1 H), 3.09 (dd, J=12.0, 3.5 Hz 1 H), 3.24 (m, 1 H), 3.65 (d, J=11.9 Hz, 1 H), 3.84 (m, 2 H), 4.28 (d, J=13.1 Hz, 1 H), 4.66 (br s, 1 H), 4.74 (s, 2 H), 6.57 (d, J=9.1 Hz, 1 H), 6.86 (d, J=7.3 Hz, 1 H), 7.17 (d, J=8.3 Hz, 1 H), 7.62-7.71 (m, 2 H), 8.40 (s, 1 H). |
| 31 | 11 | 100 | 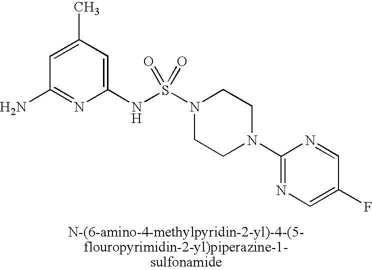 | A | ¹H NMR (400 MHz, CD₃OD) δ ppm: 2.29 (s, 3 H), 3.31-3.35 (m, 4 H), 3.80-3.90 (m, 4 H), 6.34 (s, 1 H), 6.41 (s, 1 H), 8.28 (s, 2 H). |
| 32 | 2.4 | 97 | 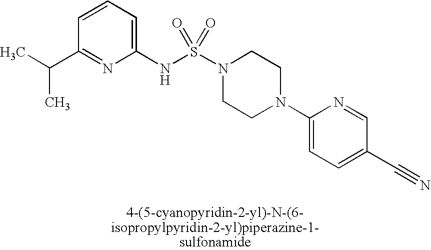 | A | ¹H NMR (400 MHz, CD₃CN) δ ppm: 1.19 (d, J=6.82 Hz, 6 H), 2.89 (m, 1 H), 3.21-3.29 (m, 4 H), 3.67-3.75 (m, 4 H), 6.72-6.80 (m, 2 H), 6.94 (d, J=8.08 Hz, 1 H), 7.55-7.63 (m, 1 H), 7.71 (dd, J=9.09, 2.27 Hz, 1 H), 8.39 (d, J=2.27 Hz, 1 H). |
| 33 | 50 | 100 | 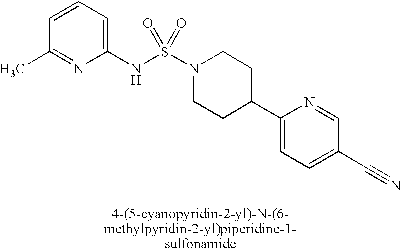 | A | ¹H NMR (400 MHz, CDCl₃) δ ppm 1.85-189 (m, 2 H), 1.94-2.05 (m ,2 H), 2.45 (s, 3 H), 2.82-2.94 (m, 3 H), 3.94 (d, J=12.13 Hz, 2 H), 6.65 (d, J=7.4 Hz, 1 H), 6.99 (d, J=8.6 Hz, 1 H), 7.28 (m, 1 H) 7.53 (dd, J=8.6, 7.4 Hz, 1 H), 7.90 (m, 1 H), 8.80 (s, 1 H). |
| 34 | 26 | 100 | 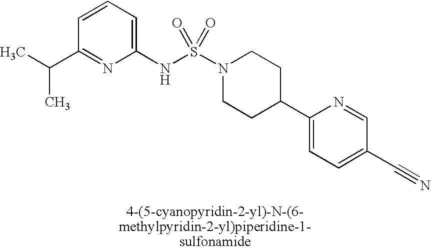 | A | ¹H NMR (400 MHz, CD₃CN) δ ppm: 1.42 (d, J=7.1 Hz, 6 H), 2.079 (d, 2 H), 3.09-3.14 (m, 2 H), 3.15-3.25 (m, 4 H), 3.97 (d, J=12.4 Hz, 2 H), 7.24 (d, J=7.8 Hz, 1 H), 7.65 (t, J=8.2 Hz, 2 H), 8.17 (t, 1 H), 8.29 (dd, J=8.3, 2.0 Hz, 1 H), 8.80 (s, 1 H). |
| 35 | 10 | 100 | 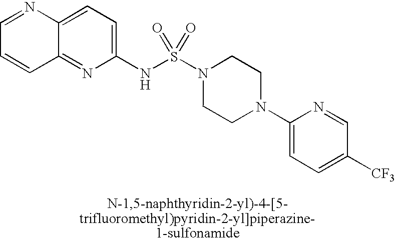 | A | ¹H NMR (400 MHz, CD₃CN) δ ppm: 3.32 (br. s, 4 H), 3.72-3.77 (m, 4 H), 6.81 (d, J=9.1 Hz, 1 H), 7.34 (d, J=9.6 Hz, 1 H), 7.57 (dd, J=8.5, 4.4 Hz, 1 H), 7.73 (dd, J=9.1, 2.5 Hz, 1 H), 7.94 (d, J=7.8 Hz, 1 H), 8.12 (d, J=9.6 Hz, 1 H), 8.39 (s, 1 H), 8.68 (d, J=3.5 Hz, 1 H). |
| 36 | NA | 89 | 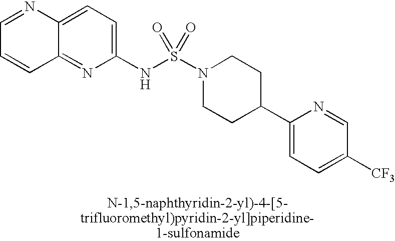 | A | ¹H NMR (400 MHz, CDCl₃) δ ppm: 1.94-2.06 (m, 4 H), 2.83-2.93 (t, J=11.6 Hz, 3 H), 3.95 (d, J=11.6 Hz, 2 H), 7.11 (d, J=7.8 Hz, 1 H), 7.28 (d, J=8.3 Hz, 1 H), 7.50 (dd, J=8.2, 4.4 Hz, 1 H), 7.71 (d, J=7.8 Hz, 1 H), 7.858 (d, J=7.8 Hz, 1 H), 8.07 (d, J=9.6 Hz, 1 H), 8.68 (d, J=3.8 Hz, 1 H), 8.78 (s, 1 H). |
| 37 | 100 | 80 | 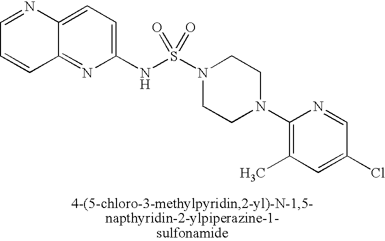 | A | ¹H NMR (400 MHz, CDCl₃) δ ppm: 2.24 (s, 3 H), 3.21 (s, 1 H), 3.23 (t, J=5.1 Hz, 3 H), 3.39 (t, 5 H), 7.10 (d, J=9.3 Hz, 1 H), 7.38 (d, J=2.3 Hz, 1 H), 7.50 (dd, J=8.3, 4.5 Hz, 1 H), 7.70 (d, J=8.1 Hz, 1 H), 8.06 (dd, J=5.9, 3.7 Hz, 2 H), 8.68 (d, J=3.3 Hz, 1 H). |
| 38 | 31 | 100 | 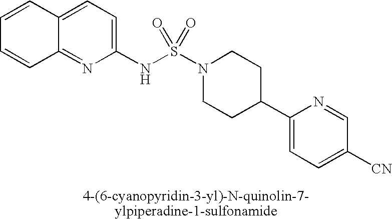 | B | ¹H NMR (400 MHz, CDCl₃) δ ppm 1.95-2.06 (m, 4 H) 2.82-2.92 (m, 3 H) 3.95 (d, J=11.87 Hz, 2 H) 6.86 (d, J=9.35 Hz, 1 H) 7.29-7.37 (m, 3 H) 7.58-7.65 (m, 2 H) 7.85 (d, J=9.35 Hz, 1 H) 7.91 (dd, J=8.21, 2.15 Hz, 1 H) 8.81 (d, J=1.52 Hz, 1 H) |
| 39 | 16 | 91 | 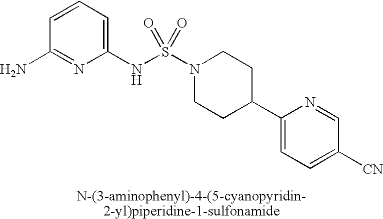 | B | ¹H NMR (400 MHz, CD₃OD) δ ppm 1.80 (qd, J=12.46, 4.04 Hz, 2 H) 1.90-1.97 (m, 2 H) 2.84-2.96 (m, 3 H) 3.89 (d, J=12.38 Hz, 2 H) 6.12 (d, J=8.08 Hz, 1 H) 6.39 (d, J=7.58 Hz, 1 H) 7.38 (t, J=8.08 Hz, 1 H) 7.45 (d, J=7.58 Hz, 1 H) 8.07 (dd, J=8.08, 2.27 Hz, 1 H) 8.81 (d, J=1.26 Hz, 1 H) |
| 40 | NA | 100 | 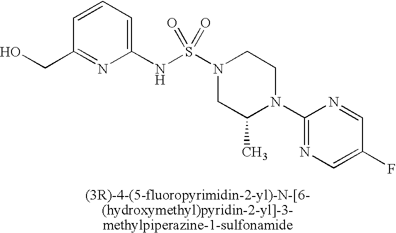 | A | ¹H NMR (400 MHz, MeOH-d₄) δ ppm: 1.12 (d, J=6.57 Hz, 3 H), 2.83-2.93 (m, 1 H), 3.03 (dd, J=11.87, 3.28 Hz, 1 H), 3.17 (td, J=12.95, 3.16 Hz, 1 H), 3.35 (br. s., 1 H). 3.61 (d, J=11.87 Hz, 1 H), 3.80 (d, J=11.62 Hz, 1 H), 4.52 (d, J=12.88 Hz, 1 H), 4.65 (s, 2 H), 7.09 (d, J=7.33 Hz, 1 H), 7.14 (d, J=8.34 |
| # 8.34 Hz, 1 H), 7.83 (t, =7.96 Hz, 1 H), 8.29 (s, 2 H) | | | | | |
| 41 | NA | 55 | 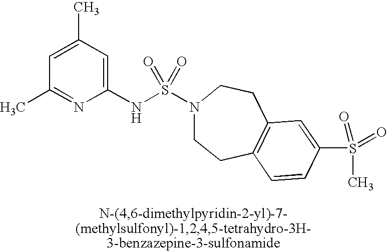 | A | ¹H NMR (400 MHz, CDCl₃) δ ppm 2.25 (s, 3 H) 2.37 (s, 3 H) 3.03 (s, 3 H) 3.08-3.13 (m, 4 H) 3.50 (dd, J=9.35, 4.04 Hz, 4 H) 6.39 (s, 1 H) 6.67 (s, 1 H) 7.29 (d, J=7.83 Hz, 1 H) 7.67 (d, J=1.77 Hz, 1 H) 7.70 (dd, J=7.83, 2.02 Hz, 1 H) |
| 42 | 17 | 89 | 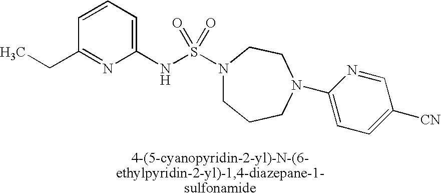 | A | ¹H NMR (400 MHz, CD₃OD) δ ppm 1.24 (t, J=7.58 Hz, 3 H) 1.88-1.94 (m, J=5.94, 5.94, 5.94, 5.94 Hz, 2 H) 2.67 (q, J=7.58 Hz, 2 H) 3.40 (t, J=5.61 Hz, 2 H) 3.56 (t, J=5.56 Hz, 2 H) 3.76 (t, J=5.68 Hz, 2 H) 3.84-3.90 (m, 2 H) 6.71 (dd, J=11.62, 8.34 Hz, 2 H) 6.83 (d, J=8.59 Hz, 1 H) 7.47 (dd, J=8.34, |
| # 7.33 Hz, 1 H) 7.64 (dd, J=9.09, 2.27 Hz, 1 H) 8.30 (d, J=1.52 Hz, 1 H) | | | | | |
| 43 | 12 | 93 | 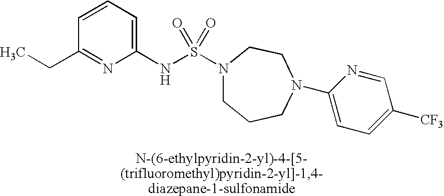 | A | ¹H NMR (400 MHz, CDCl₃) δ ppm 1.25 (td, J=7.58, 1.01 Hz, 3 H) 1.98-2.06 (m, J=5.98, 5.98, 5.98, 5.81 Hz, 2 H) 2.67 (q, J=7.75 Hz, 2 H) 3.35 (t, J=5.81 Hz, 2 H) 3.56 (t, J=4.67 Hz, 2 H) 3.74 (t, J=6.06 Hz, 2 H) 3.85-3.92 (m, 2 H) 6.47 (d, J=9.10 Hz, 1 H) 6.64 (d, J=7.33 Hz, 1 H) 6.79 (d, J=7.58 Hz, |
| # 1 H) 7.46 (t, J=7.83 Hz, 1 H) 7.57 (d, J=8.84 Hz, 1 H) 8.31 (d, J=0.76 Hz, 1 H) | | | | | |
| 44 | 34 | 72 | 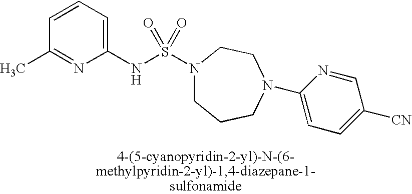 | A | ¹H NMR (400 MHz, CDCl₃) δ ppm 1.97-2.05 (m, 2 H) 2.42 (s, 3 H) 3.38 (t, J=6.06 Hz, 2 H) 3.53-3.59 (m, 2 H) 3.76 (t, J=6.19 Hz, 2 H) 3.87-3.94 (m, 2 H) 6.47 (d, J=9.09 Hz, 1 H) 6.67 (d, J=7.33 Hz, 1 H) 6.81 (d, J=8.34 Hz, 1 H) 7.45-7.51 (m, 1 H) 7.57 (dd, J=8.84, 2.27 Hz, 1 H) 8.34 (d, J=1.77 Hz, 1 H) |
| 45 | 53 | 73 | 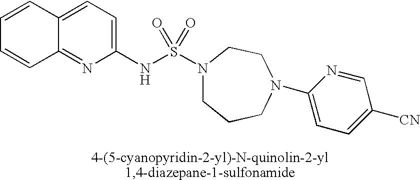 | A | ¹H NMR (400 MHz, CDCl₃) δ ppm 2.01-2.08 (m, 2 H) 3.37-3.42 (m, 2 H) 3.55-3.60 (m, 2 H) 3.80 (t, J=5.81 Hz, 2 H) 3.89-3.95 (m, 2 H) 6.47 (d, J=8.84 Hz, 1 H) 6.62 (d, J=8.84 Hz, 1 H) 7.29-7.37 (m, 2 H) 7.53 (dd, J=8.97, 2.40 Hz, 1 H) 7.56-7.63 (m, 2 H) 7.78 (d, J=9.35 Hz, 1 H) 8.28 (d, J=2.02 Hz, 1 H) |
| 46 | <1 | 62 | 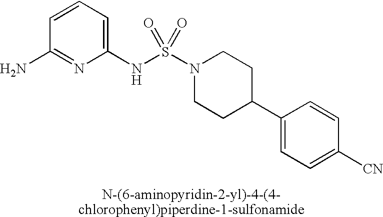 | B | ¹H NMR (400 MHz, CD₃OD) δ ppm 1.58-1.69 (m, 2 H) 1.80 (dd, J=12.51, 1.39 Hz, 2 H) 2.54-2.63 (m, 1 H) 2.89 (td, J=12.38, 2.27 Hz, 2 H) 3.84-3.91 (m, 2 H) 6.13 (d, J=8.08 Hz, 1 H) 6.39 (dd, J=7.96, 0.63 Hz, 1 H) 7.14-7.18 (m, 2 H) 7.23-7.27 (m, 2 H) 7.38 (t, J=8.08 Hz, 1 H) |
| 47 | NA | 100 | 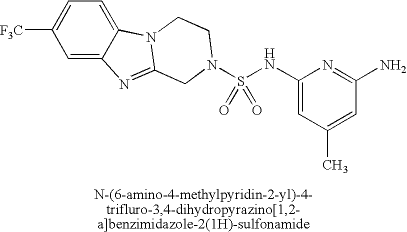 | A | ¹H NMR (400 MHz, CDCl₃) δ ppm 2.13-2.26 (m, 3 H) 3.80-3.93 (m, 2 H) 4.20-4.29 (m, 2 H) 4.72-4.83 (m, 2 H) 5.72 (s, 2 H) 5.77 (s, 1 H) 6.67 (s, 1 H) 7.29 (s, 1 H) 7.43 (dd, J=8.46, 1.14 Hz, 1 H) 7.89 (s, 1 H) |
| 48 | NA | 100 | 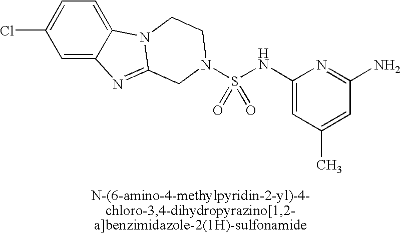 | A | ¹H NMR (400 MHz, CDCl₃) δ ppm 2.14-2.24 (m, 3 H) 3.80-3.93 (m, 2 H) 4.20-4.34 (m, 2 H) 4.72-4.82 (m, 2 H) 5.72 (s, 2 H) 5.77 (s, 1 H) 6.67 (s, 1 H) 7.29 (s, 1 H) 7.43 (dd, J=8.46, 1.14 Hz, 1 H) 7.89 (s, 1 H) |
| 49 | NA | 100 | 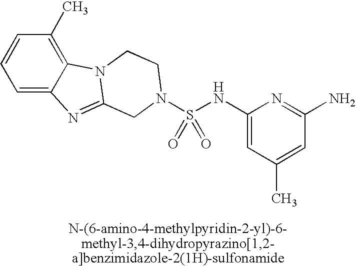 | A | ¹H NMR (400 MHz, DMSO-d₆) δ ppm 7.44 (d, J=8.08 Hz, 1 H) 7.11 (t, J=7.58 Hz, 1 H) 6.99 (d, J=7.33 Hz, 1 H) 6.19 (s, 1 H) 5.81 (s, 1 H) 4.61 (t, J=5.31 Hz, 2 H) 4.51 (t, 1 H) 3.71 (s, 2 H) 2.69-2.78 (m, 3 H) 2.15 (s, 3 H) |
| 50 | NA | 100 | 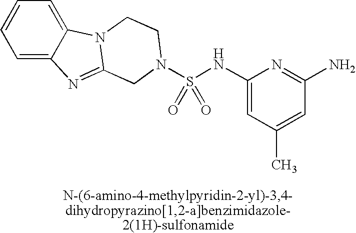 | A | ¹H NMR (400 MHz, CDCl₃) δ ppm 2.18 (s, 3 H) 3.88 (t, J=5.43 Hz, 2 H) 4.22 (t, J=5.43 Hz, 2 H) 4.78 (s, 2 H) 5.77 (s, 1 H) 6.59 (s, 1 H) 7.21-7.34 (m, 3 H) 7.68 (s, 1 H) |
| 51 | NA | 38 | 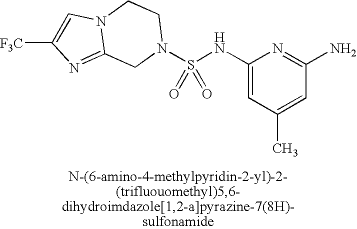 | A | ¹H NMR (400 MHz, , CDCl₃) δ ppm 2.23 (s, 3 H) 3.70 (t, J=5.43 Hz, 2 H) 4.16 (t, J=5.43 Hz, 1 H) 4.53 (s, 2 H) 5.77 (s, 2 H) 5.84 (s, 1 H) 6.70 (s, 1 H) 7.15 (d, J=1.26 HZ, 1 H) |
| 52 | NA | 100 | 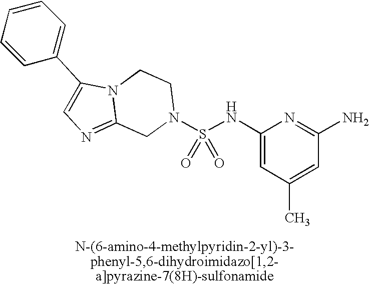 | A | ¹H NMR (400 MHz, CDCl₃) δ ppm 2.22 (s, 3 H) 3.59-3.74 (m, 2 H) 4.11-4.25 (m, 2 H) 5.87 (s, 1 H) 6.60 (s, 1 H) 6.94 (s, 1 H) 7.33-7.50 (m, 3 H) 7.55-7.66 (m, 2 H) |
| 53 | NA | 100 | 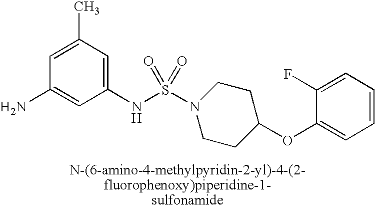 | A | ¹H NMR (400 MHz, CDCl₃) δ ppm 6.83-7.03 (m, 4 H) 6.45 (s, 1 H) 5.91 (s, 1 H) 4.27-4.34 (m, 1 H) 3.45-3.55 (m, 2 H) 3.17-3.27 (m, 2 H) 2.15 (s, 3 H) 1.77-1.95 (m, 4 H) |
| 54 | NA | 100 | 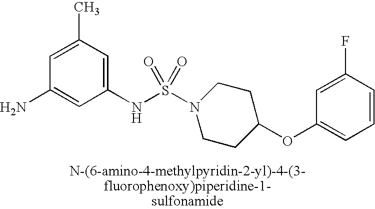 | A | ¹H NMR (400 MHz, CDCl₃) δ ppm 7.09-7.16 (m, 1 H) 6.55-6.61 (m, 2 H) 6.48-6.54 (m, 1 H) 6.45 (s, 1 H) 5.93 (s, 1 H) 4.34 (d, J=3.28 Hz, 1 H) 3.39-3.48 (m, 2 H) 3.26 (s, 2 H) 2.16 (s, 3 H) 1.72-1.95 (m, 4 H) |
| 55 | NA | 92 | 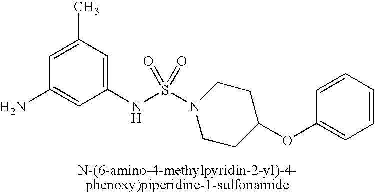 | A | ¹H NMR (400 MHz, CDCl₃) δ ppm 2.28 (s, 3 H) 3.38 (s, 3 H) 3.54 (d, J=23.49 Hz, 3 H) 4.47 (s, 1 H) 6.05 (s, 1 H) 6.59 (s, 1 H) 6.84-7.07 (m, 5 H) |
| 56 | NA | 100 | 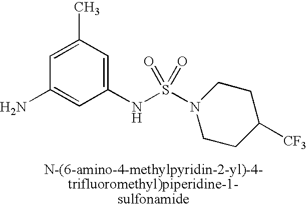 | A | ¹H NMR (400 MHz, CDCl₃) δ ppm 1.49-1.68 (m, 2 H) 1.88 (s, 2 H) 1.99-2.15 (m, 1 H) 2.66-2.82 (m, 2 H) 3.89 (d, J=12.13 Hz, 2 H) 5.43 (s, 2 H) 5.88 (s, 1 H) 6.58 (s, 1 H) |
| 57 | 56 | 85 | 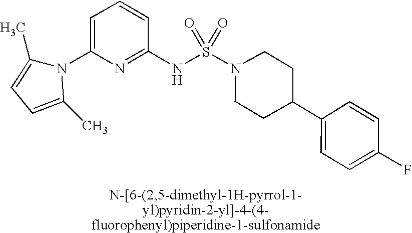 | A | ¹H NMR (400 MHz, CDCl₃) δ ppm 1.56 (s, 6 H) 1.68 (qd, J=12.76, 4.17 Hz, 2 H) 1.83-1.91 (m, 2 H) 2.50-2.59 (m, 1 H) 2.92 (td, J=12.44, 2.40 Hz, 2 H) 3.94-4.00 (m, 2 H) 5.92 (s, 2 H) 6.95-7.01 (m, 3 H) 7.06-7.13 (m, 2 H) 7.33 (d, J=8.08 Hz, 1 H) 7.83 (t, J=7.96 Hz, 1 H) |
| 58 | NA | 100 | 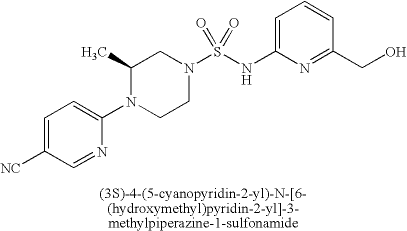 | B | ¹H NMR (400 MHz, DMSO-d₆) δ ppm 8.50 (d, J=1.76 Hz, 1 H), 7.87 (dd, J=9.19, 2.39 Hz, 1 H), 7.69-7.75 (m, 1 H), 6.99 (d, J=8.06 Hz, 2 H), 6.88 (d, J=9.06 Hz, 1 H), 5.64 (s, 3 H), 4.67-4.78 (m, 1 H), 4.45 (s, 2 H), 4.26-4.35 (m, 1 H), 3.65-3.74 (m, 1 H), 3.43-3.52 (m, 1 H), 3.05-3.17 (m, 1 H), |
| # 2.92-3.00 (m, 1 H), 2.78-2.89 (m, 1 H), 1.05 (d, J=6.55 Hz, 3 H) | | | | | |
| 59 | NA | 94.2 | 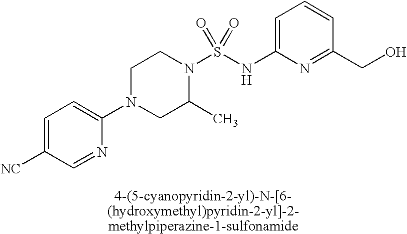 | B | ¹H NMR (400 MHz, DMSO-d₆) δ ppm 8.45 (d, J=2.27 Hz, 1 H), 7.84 (dd, J=9.06, 2.52 Hz, 1 H), 7.70 (t, J=7.93 Hz, 1 H), 7.03 (d, J=7.30 Hz, 1 H), 6.88 (d, J=8.81 Hz, 2 H), 4.44 (s, 2 H), 4.10-4.30 (m, 3 H), 3.57-3.68 (m, 1 H), 3.24-3.35 (m, 1 H), 3.13-3.22 (m, 1 H), 2.89-3.00 (m, 1 H), 1.07 (d, |
| # J=6.55 Hz, 3 H) | | | | | |
| 60 | ND | 7 | 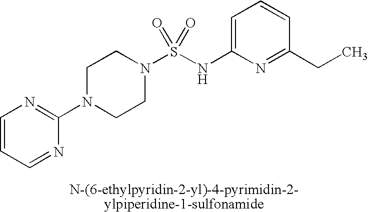 | B | ¹H NMR (400 MHz, CDCl₃, ppm), δ: 8.98 (br. s., 1 H), 8.74 (d, J=5.04 Hz, 2 H), 8.00 (t, J=8.18 Hz, 1 H), 7.65 (d, J=8.81 Hz, 1 H), 7.23 (t, J=4.91 Hz, 1 H), 7.02 (d, J=7.55 Hz, 1 H), 3.92-4.02 4.02 (m, 2 H), 3.17-3.29 (m, 2 H), 3.05-3.16 (m, 1 H), 2.97 (q, J=7.55 Hz, 2 H), 2.12-2.22 (m, 2 H), 1.97-2.11 |
| # (m, 2 H), 1.37 (t, J≦7.55 Hz, 3 H). | | | | | |
| 61 | ND | 0 | 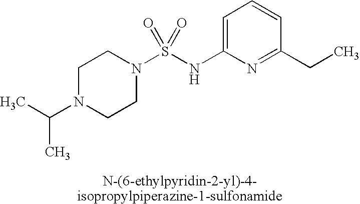 | B | ¹H NMR (400 MHz, CDCl₃, ppm), δ: 7.71-7.78 (m, 1 H), 7.23-7.28 (m, 1 H), 6.71 (d, J=7.30 Hz, 1 H), 5.37 (s, 1 H), 3.77-4.01 (m, 2 H), 3.33-3.67 (m, 5 H), 3.05-3.30 (m, 2 H), 2.82 (q, J=7.55 Hz, 2 H), 1.38 (d, J=6.80 Hz, 6 H), 1.33 (t, J=7.68 Hz, 3 H). |
| 62 | ND | 0 | 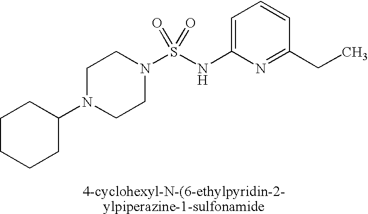 | B | ¹H NMR (400 MHz, CDCl₃, ppm), δ: 7.79-7.87 (m, 1 H), 7.62 (br. s., 1 H), 7.36 (d, J=8.81 Hz, 1 H), 6.82 (d, J=7.30 Hz, 1 H), 3.82-3.99 (m, 2 H), 3.41-3.67 (m, 4 H), 3.06-3.31 (m, 3 H), 2.86 (q, J=7.55 Hz, 2 H), 2.06-2.17 (m, 2 H), 1.88-2.00 (m, 2 H), 1.65-1.78 (m, 1 H), 1.39-1.54 (m, 2 H), 1.24-1.39 (m, 5 H), 1.07-1.23 (m, 1 H). |
| 63 | ND | 40 | 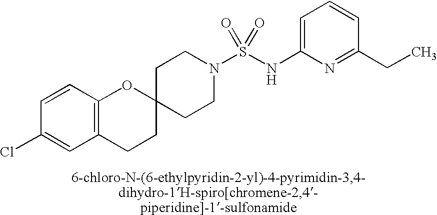 | B | ¹H NMR (400 MHz, CDCl₃, ppm), δ: 9.72 (br. s., 1 H), 7.54 (t, J=7.93 Hz, 1 H), 6.95-7.06 (m, 3 H), 6.71 (d, J=9.57 Hz, 1 H), 6.65 (d, J=7.30 Hz, 1 H), 3.54-3.66 (m, 2 H), 3.12-3.25 (m, 2 H), 2.65-2.79 (m, 4 H), 1.72-1.88 (m, 4 H), 1.58-1.71 (m, 2 H), 1.29 (t, J=7.55 Hz, 3 H). |
| 64 | ND | 5.4 | 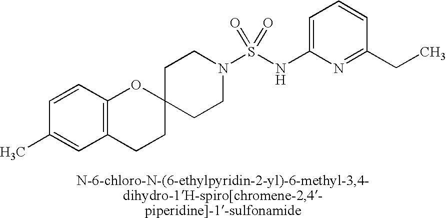 | B | ¹H NMR (400 MHz, CDCl₃, ppm), δ: 9.37 (br. s., 1 H), 7.53 (t, J=7.93 Hz, 1 H), 6.99 (d, J=8.56 Hz, 1 H), 6.83-6.91 (m, 2 H), 6.67 (t, J=8.06 Hz, 2 H), 3.54-3.64 (m, 2 H), 3.15-3.27 (m, 2 H), 2.66-2.77 (m, 4 H), 2.24 (s, 3 H), 1.79-1.89 (m, 2 H), 1.75 (t, J=6.67 Hz, 2 H), 1.58-1.69 (m, 2 H), 1.29 (t, J=7.68 Hz, 3 H). |
| 65 | ND | 4.7 | 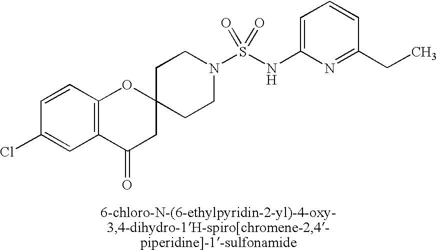 | B | ¹H NMR (400 MHz, CDCl₃, ppm), δ: 10.10 (br. s., 1 H), 7.80 (d, J=2.52 Hz, 1 H), 7.56 (t, J=7.93 Hz, 1 H), 7.42 (m, 1 H), 6.98 (d, J=8.56 Hz, 1 H), 6.93 (d, J=8.81 Hz, 1 H), 6.63 (d, J=7.05 Hz, 1 H), 3.54-3.65 (m, 2 H), 3.14-3.26 (m, 2 H), 2.66-2.79 (m, 4 H), 2.01-2.13 (m, 2 H), 1.69-1.82 (m, 2 H), 1.29 (t, J=7.30 Hz, 3 H). |
| 66 | ND | 42.8 | 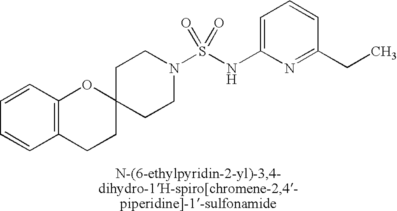 | B | ¹H NMR (400 MHz, CDCl₃, ppm), δ: 9.53 (br. s., 1 H), 7.54 (t, J=7.93 Hz, 1 H), 6.96-7.11 (m, 3 H), 6.83 (t, J=7.43 Hz, 1 H), 6.77 (d, J=8.06 Hz, 1 H), 6.66 (d, J=7.30 Hz, 1 H), 3.54-3.65 (m, 2 H), 3.17-3.28 (m, 2 H), 2.67-2.79 (m, 4 H), 1.80-1.90 (m, 2 H), 1.77 (t, J=6.80 Hz, 2 H), 1.59-1.71 (m, 2 H), 1.29 (t, J=7.68 Hz, 3 H). |
| 67 | ND | 62.6 | 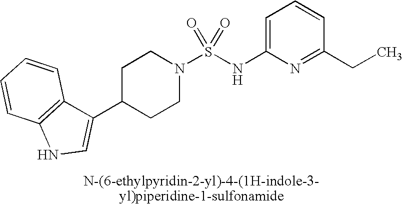 | B | ¹H NMR (400 MHz, CDCl₃, ppm), δ: 9.81 (br. s., 1 H), 8.25 (s, H), 7.53 (t, J=7.93 Hz, 2 H), 7.35 (d, J=8.06 Hz, 1 H), 7.16 (t, J=7.55 Hz, 1 H), 7.06 (t, J=7.43 Hz, 1 H), 7.02 (d, J=8.56 Hz, 1 H), 6.90 (d, J=2.27 Hz, 1 H), 6.63 (d, J=7.30 Hz, 1 H), 3.86-3.95 (m, 2 H), 2.84-2.99 (m, 3 H), 2.71 (q, J=7.64 |
| # Hz, 2 H), 2.02-2.11 (m, 2 H), 1.71-1.85 (m, 2 H), 1.27 (t, J=7.55 Hz, 3 H). | | | | | |
| 68 | 5.4 | 96.6 | 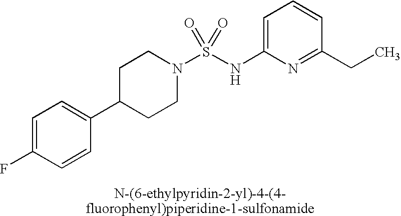 | B | ¹H NMR (400 MHz, CDCl₃, ppm), δ: 9.68 (br. s., 1 H), 7.55 (t, J=7.93 Hz, 1 H), 7.07-7.15 (m, 2 H), 6.93-7.03 (m, 3 H), 6.65 (d, J=7.30 Hz, 1 H), 3.87-3.96 (m, 2 H), 2.80-2.91 (m, 2 H), 2.73 (q, J7.55 Hz, 2 H), 2.49-2.61 (m, 1 H), 1.81-1.91 (m, 2 H), 1.65-1.79 (m, 2 H), 1.29 (t, J=7.68 Hz, 3 H). |
| 69 | ND | 5.6 | 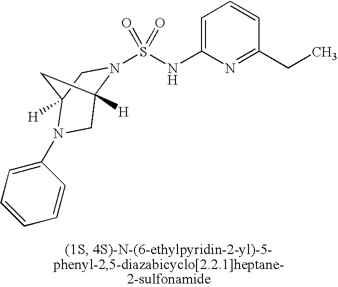 | B | ¹H NMR (400 MHz, CDCl₃, ppm), δ: 9.55 (br. s., 1 H), 7.38-7.47 (m, 1 H), 7.18 (t, J=7.93 Hz, 2 H), 6.77 (d, J=8.81 Hz, 1 H), 6.68 (t, J=7.30 Hz, 1 H), 6.45-6.59 (m, 3 H), 4.54 (s, 1 H), 4.38 (s, 1 H), 3.37-3.55 (m, 4 H), 2.63 (q, J=7.72 Hz, 2 H), 1.93-2.10 (m, 2 H), 1.24 (t, J=7.68 Hz, 3 H). |

### Examples 70-202

Examples 70 to 202 are shown in Table 2 below. To a corresponding oxazolidine-3-sulfonamide intermediate (any of intermediate 2(i), 3(i) or 12(i) as prepared above) (320 μL, 1.0 eq., 0.25 M in anhydrous DMF) was added a compound of formula NHR²R³ (320 μL, 1.0 eq., 0.25 M in anhydrous DMF) and DIEA (28 μL, 0.16 mmol, 2.0 eq., neat DIEA) in a 10×95 mm test tube containing a stir bar. The reaction mixture was heated at 100° C. for 16 h under reflux and then cooled to ambient temperature. Solvents and volatiles were evaporated in vacuo, and the resulting residues were dissolved in DMSO to give a 0.0572 M solution. The crude reaction mixtures were analyzed by HPLC, filtered through Whatman GF/F Unifilter (#7700-7210) followed by purification by reverse phase HPLC (LC/MS/UV). HPLC fractions were collected in 23 ml pre-tared tubes and evaporated to dryness. Dried product was weighed and dissolved in DMSO at concentrations of 30 mM or 10 mM. The HPLC eluent contained TFA. As a consequence, some compounds were isolated as TFA salts. Products were then analyzed using HPLC (LC/MS/UV/ELSD). Products determined to be at least 85% pure by two or more detector channels were registered and made available for screening. The NHR²R³ groups were either commercially available or prepared by routine methods available to one skilled in the art.

TABLE 2

| | | | | % | |
|---|---|---|---|---|---|
| | | | | Inh | |
| | | | | @ | |
| | | | Klapp | 0.1 | Mass |
| Ex. # | Structure | Name | (nM) | mM | (m/z) |
| 70 | 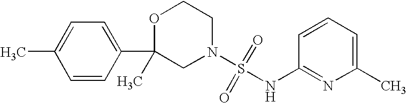 | 2-methyl-2-(4-methylphenyl)- N-(6-methylpyridin-2- yl)morpholine-4-sulfonamide | | 85.3 | 362.0 |
| 71 | 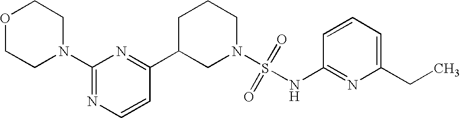 | N-(6-ethylpyridin-2-yl)-3-(2- morpholin-4-ylpyrimidin- 4-yl)pipendine-1-sulfonamide (TFA Salt) | 23 | 92.7 | 433.1 |
| 72 | 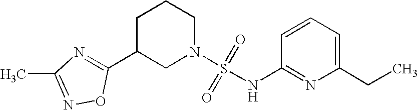 | N-(6-ethylpyridin-2-yl)-3-(2- methyl-124-oxadiazol-5- yl)piperidine-1-sulfonamide (TFA Salt) | | 56.7 | 352.1 |
| 73 | 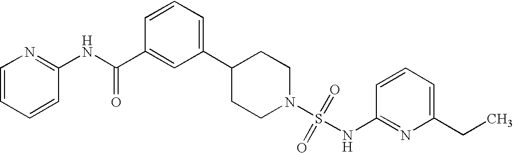 | 3-(1-{[(6-ethylpyridin-2- yl)amino]sulfonyl}pipendin- 4-yl)-N-pyridin-2- ylbenzamide (TFA Salt) | | 49.6 | 466.1 |
| 74 | 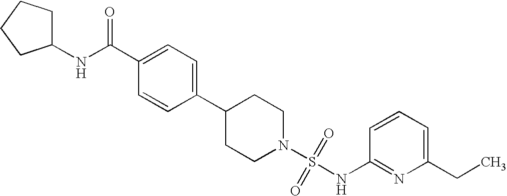 | N-cyclopentyl-4-(1-{[(6- ethylpyrin-2- yl)amino]sulfonyl}pipendin- 4-yl)benzamide (TFA Salt) | 9.6 | 88.4 | 457.1 |
| 75 | 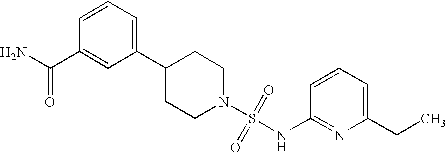 | 3-(1-{[(6-ethylpyridin-2- yl)amino]sulfonyl}piperidin- 4-yl)benzamide (TFA Salt) | | 45.6 | 389.0 |
| 76 | 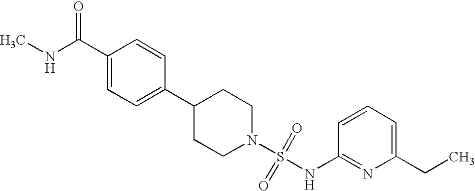 | 4-(1-{[(6-ethylpyridin-2- yl)amino]sulfonyl}piperidin- 4-yl)-N-methylbenzamide (TFA Salt) | | 34.4 | 403.1 |
| 77 | 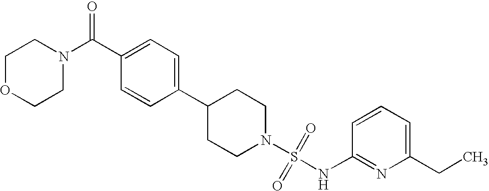 | N-(6-ethylpyridin-2-yl)-4-[4- (morpholin-4- ylcarbonyl)phenyl]piperi- din-1-sulfonamide (TFA Salt) | | 48.4 | 459.1 |
| 78 | 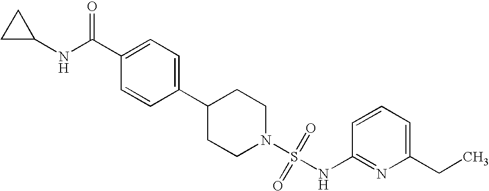 | N-cyclopropyl-4-(1- {[(6-ethylpyridin-2- yl)amino]sulfonyl}piperi- din-4-yl)benzamide (TFA Salt) | | 64.2 | 429.1 |
| 79 | 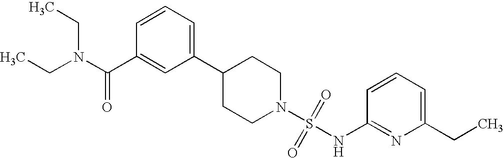 | NN-diethy-3-(1-{[(6- ethylpyridin-2- yl)amino]sulfonyl}piperidin- 4-yl)benzamide (TFA Salt) | | 51.1 | 445.1 |
| 80 | 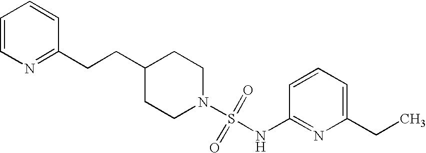 | N-(6-ethylpyridin-2-yl)-4- (2-pyridin-2- ylethyl)piperidine-1- sulfonamide (TFA Salt) | 5.6 | 93.8 | 375.1 |
| 81 | 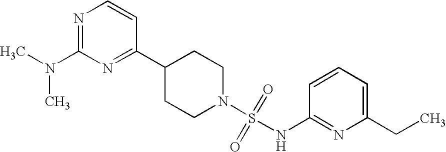 | 4-[2-(dimethylamino)pyrimi- din-4-yl]-N-(6-ethylpyridin-2- yl)piperidine-1-sulfonamide (TFA Salt) | 230 | 86.8 | 391.1 |
| 82 | 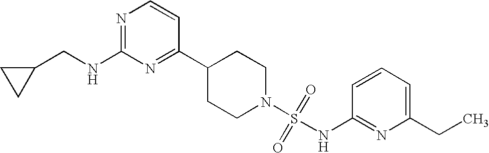 | 4-(2-{2- [(cyclopropylmethyl)- amino]pyrimidin-4-yl}ethyl)- N-(6-ethylpyridin-2- yl)piperidine-1-sulfonamide (TFA Salt) | 11 | 100 | 445.1 |
| 83 | 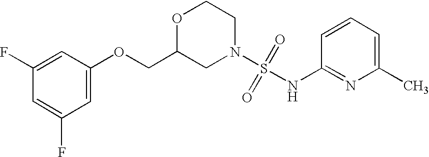 | 2-[(35-difluorophenoxy)meth- yl]-N-(6-methylpyridin- 2-yl)morpholine-4- sulfonamide | 110 | 71.2 | 400.0 |
| 84 | 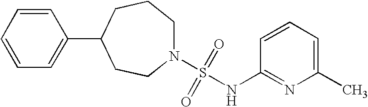 | N-(6-methylpyridin-2-yl)-4- phenylazepane-1-sulfonamide (TFA Salt) | 8.2 | 100 | 346.1 |
| 85 | 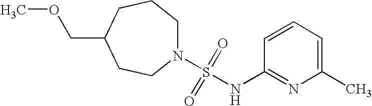 | 4-(methoxymethyl)-N-(6- methylpyridin-2-yl)azepane- yl)azepane-1-sulfonamide (TFA Salt) | | 553 | 314.0 |
| 86 | 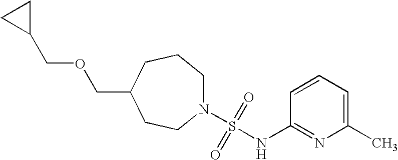 | 4-[(cyclopropylmethoxy)- methyl]-N-(6-methylpyridin- 2-yl)azepane-1-sulfonamide (TFA Salt) | 98 | 87.6 | 354.1 |
| 87 | 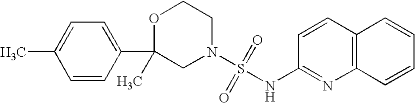 | 2-methyl-2-(4-methylphenyl)- N-quinolin-2-ylmorpholine-4- sulfonamide | 25 | 85.2 | 398.2 |
| 88 | 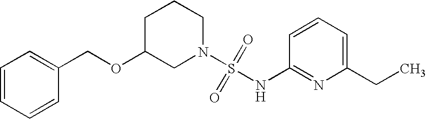 | 3-(benzyloxy)-N-(6- ethylpyridin-2-yl)piperidine- 1-sulfonamide (TFA Salt) | 52 | 77.1 | 376.1 |
| 89 | 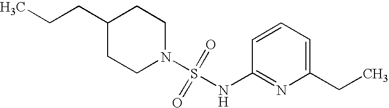 | N-(6-ethylpyridin-2-yl)-4- propylpiperidine-1- sulfonamide (TFA Salt) | 1 | 100 | 312.2 |
| 90 | 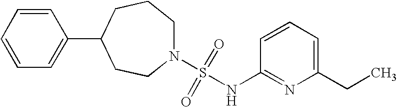 | N-(6-ethylpyridin-2-yl)-4- phenylazepane-1-sulfonamide (TFA Salt) | 18 | 90.9 | 360.1 |
| 91 | 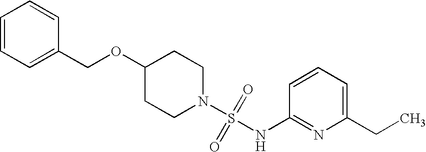 | 4-(benzyloxy)-N-(6- ethylpyridin-2-yl)piperidine- 1-sulfonamide (TFA Salt) | 45 | 75.9 | 376.1 |
| 92 | 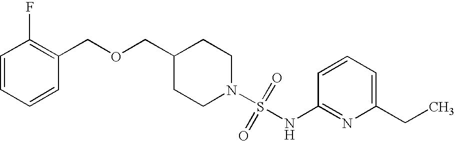 | N-(6-ethylpyridin-2-yl)-4- {[(2-fluorobenzyl)oxy]meth- yl}piperidine-1-sulfonamide (TFA Salt) | | 67.5 | 408.1 |
| 93 | 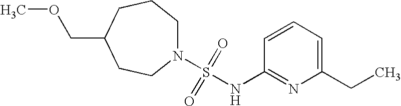 | N-(6-ethylpyridin-2-yl)-4- (methoxymethyl)azepane-1- sulfonamide (TFA Salt) | 110 | 69.7 | 328.1 |
| 94 | 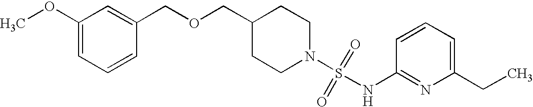 | N-(6-ethylpyridin-2-yl)-4- {[(3-methoxybenzyl)oxy]- methyl}pipendine-1- sulfonamide (TFA Salt) | | 40.7 | 420.1 |
| 95 | 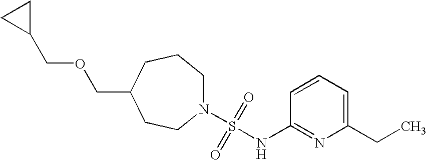 | 4-[(cyclopropylmethoxy)- methyl]-N-(6-ethylpyridin-2- yl)azepane-1-sulfonamide (TFA Salt) | 82 | 86.2 | 368.2 |
| 96 | 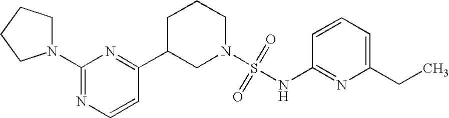 | N-(6-ethylpyridin-2-yl)-3-(2- pyrrolidin-1-ylpyrimidin-4- yl)piperidine-1-sulfanamide (TFA Salt) | 61 | 71.6 | 417.2 |
| 97 | 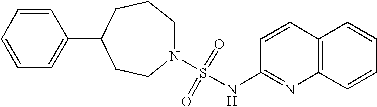 | 4-phenyl-N-quinolin-2- ylazepane-1-sulfonamide | 6.3 | 100 | 382.1 |
| 98 | 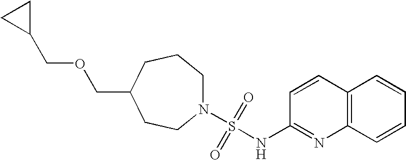 | 4-[(cyclapropylmethoxy)meth- yl]-N-quinolin-2-ylazepane-1- sulfonamide | 28 | 92.4 | 390.2 |
| 99 | 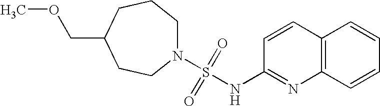 | 4-(methoxymethyl)-N- quinolin-2-ylazepane-1- sulfonamide | 63 | 77.6 | 350.2 |
| 100 | 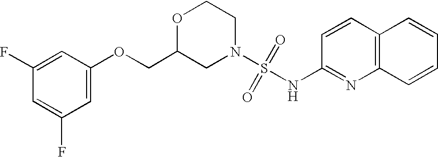 | 2-[(35-difluorophenoxy)meth- yl]-N-quinolin-2- ylmorpholine-4-sulfonamide | 59 | 90.6 | 436.1 |
| 101 | 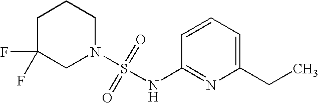 | N-(6-ethylpyridin-2-yl)-33- difluoropiperidine-1- sulfonamide (TFA Salt) | 34 | 78.3 | 306.1 |
| 102 | 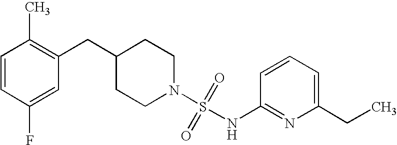 | N-(6-ethylpyridin-2-yl)-4-(5- fluoro-2- methylbenzyl)piperidine-1- sulfonamide (TFA Salt) | 5.4 | 100 | 392.2 |
| 103 | 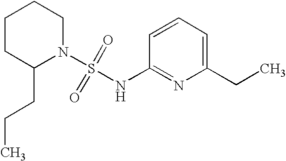 | N-(6-ethylpyridin-2-yl)-2- propylpiperidine-1- sulfonamide (TFA Salt) | 24 | 82.2 | 312.2 |
| 104 | 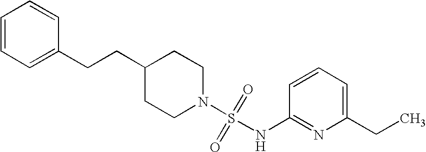 | N-(6-ethylpyridin-2-yl)-4-(2- phenylethyl)piperidine-1- sulfonamide (TFA Salt) | 6.9 | 100 | 374.2 |
| 105 | 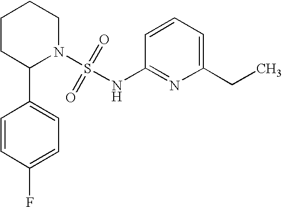 | N-(6-ethylpyridin-2-yl)-2-(4- fluorophenyl)piperidine-1- sulfonamide (TFA Salt) | 8.2 | 90.1 | 364.1 |
| 106 | 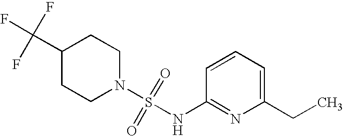 | N-(6-ethylpyridin-2-yl)-4- (trifluoromethyl)piperidine-1- sulfonamide (TFA Salt) | 7 | 100 | 338.0 |
| 107 | 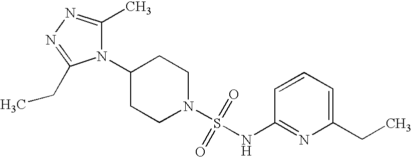 | 4-(3-ethyl-5-methyl-4H-124- triazol-4-yl)-N-(6-ethyl- pyridin-2-yl)piperidine-1- sulfonamide (TFA Salt) | | 0 | 379.1 |
| 108 | 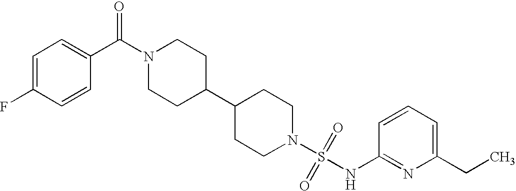 | N-(6-ethylpyridin-2-yl)-1′-(4- fluorobenzoyl)-44′- bipiperidine-1- sulfonamide (TFA Salt) | 60 | 89 | 475.1 |
| 109 | 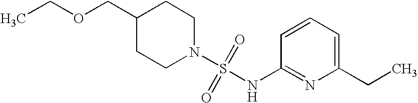 | 4-(ethoxymethyl)-N-(6- ethylpyridin-2-yl)pipendine-1- sulfonamide (TFA Salt) | | 58.7 | 328.1 |
| 110 | 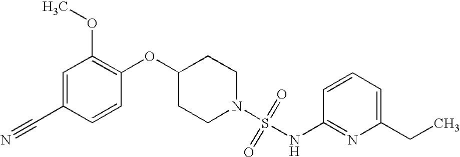 | 4-(4-cyano-2-methoxy- phenoxy)-N-(6-ethylpyridin-2- yl)piperidine-1-sulfonamide (TFA Salt) | | 8 | 417.1 |
| 111 | 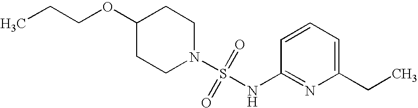 | N-(6-ethylpyridin-2-yl)-4- propoxypiperidine-1- sulfonamide (TFA Salt) | | 50.8 | 328.1 |
| 112 | 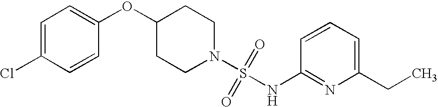 | 4-(4-chlorophenoxy)-N-(6- ethylpyridin-2-yl)pipendine-1- sulfonamide (TFA Salt) | 18 | 100 | 396.1 |
| 113 | 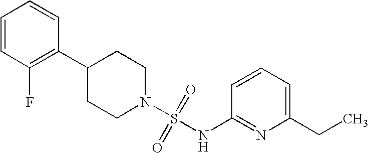 | N-(6-ethylpyridin-2-yl)-4-(2- fluorophenyl)piperidine-1- sulfonamide (TFA Salt) | 16 | 89.2 | 364.1 |
| 114 | 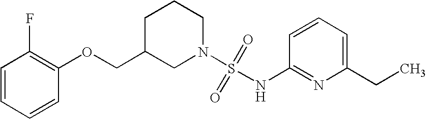 | N-(6-ethylpyridin-2-yl)-3-[(2- fluorophenoxy)methyl]piperi- dine-1-sulfonamide (TFA Salt) | 15 | 97.4 | 394.1 |
| 115 | 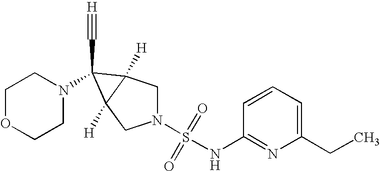 | (1R*5S*6S*)-6-cyano-N-(6- ethylpyridin-2-yl)-6- morpholin-4-yl-3- azabicyclo[3.1.0]hexane-3- sulfonamide (TFA Salt) | | 0 | 378.1 |
| 116 | 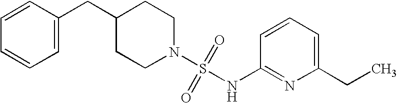 | 4-benzyl-N-(6-ethylpyridin-2- yl)piperidine-1-sulfonamide (TFA Salt) | 18 | 100 | 360.1 |
| 117 | 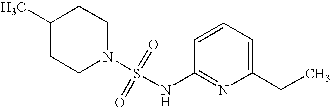 | N-(6-ethylpyridin-2-yl)-4- methylpiperidine-1- sulfonamide (TFA Salt) | 45 | 65.2 | 284.2 |
| 118 | 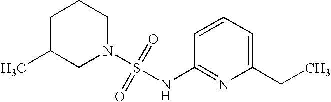 | N-(6-ethylpyridin-2-yl)-3- methylpiperidine-1- sulfonamide (TFA Salt) | 59 | 73.6 | 284.2 |
| 119 | 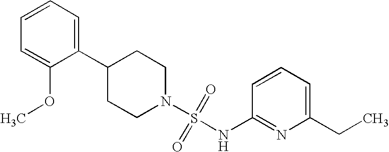 | N-(6-ethylpyridin-2-yl)-4-(2- methoxyphenyl)pipendine-1- sulfonamide (TFA Salt) | 37 | 93.1 | 376.1 |
| 120 | 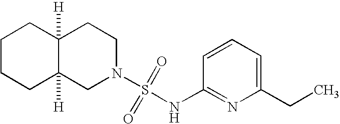 | (4aS*8aS*)-N-(6- ethylpyridin-2-yl)octahydro- isoquinoline-2(1H)- sulfonamide | 13 | 100 | 324.1 |
| 121 | 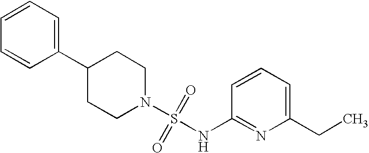 | N-(6-ethylpyridin-2-yl)-4- phenylpiperidine-1- sulfanamide (TFA Salt) | 3.5 | 100 | 346.1 |
| 122 | 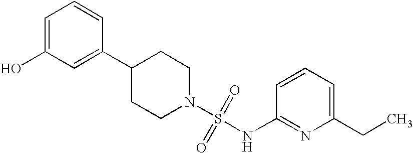 | N-(6-ethylpyridin-2-yl)-4-(3- hydroxyphenyl)piperidine-1- sulfonamide (TFA Salt) | 11 | 91.7 | 362.2 |
| 123 | 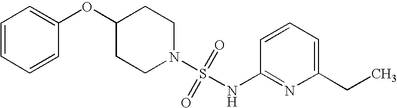 | N-(6-ethylpyridin-2-yl)-4- phenoxypiperidine-1- sulfanamide (TFA Salt) | 22 | 100 | 362.2 |
| 124 | 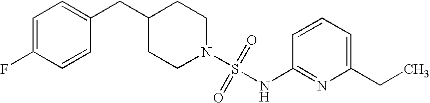 | N-(6-ethylpyridin-2-yl)-4-(4- fluorobenzyl)pipendine-1- sulfonamide (TFA Salt) | 7 | 88.4 | 378.1 |
| 125 | 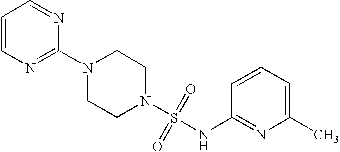 | N-(6-methylpyridin-2-yl)-4- pyrimidin-2-ylpiperazine-1- sulfonamide (TFA Salt) | 28.5 | 75.1 | 335.0 |
| 126 | 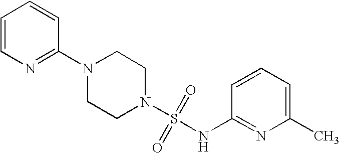 | N-(6-methylpyridin-2-yl)-4- pyridin-2-ylpiperazine-1- sulfonamide (TFA Salt) | | 57.8 | 334.0 |
| 127 | 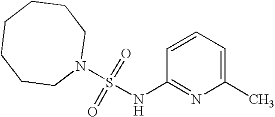 | N-(6-methylpyridin-2- yl)azocane-1-sulfonamide | 25 | 91.8 | 283.4 |
| 128 | 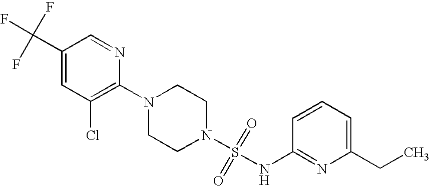 | 4-[3-chloro-5- (trifluoromethyl)pyridin-2- yl]-N-(6-methylpyridin-2- yl)piperazine-1-sulfonamide (TFA Salt) | 13 | 100 | 436.0 |
| 129 | 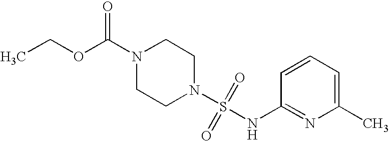 | ethyl 4-{[(6-methylpyridin-2- yl)amino]sulfonyl}piperazine- 1-carboxylate (TFA Salt) | 28 | 86.6 | 329.0 |
| 130 | 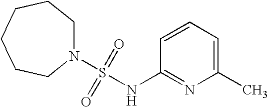 | N-(6-methylpyridin-2- yl)azepane-1-sulfonamide (TFA Salt) | | 56.7 | 270.1 |
| 131 | 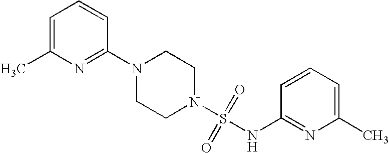 | N4-bis(6-methylpyridin-2- yl)piperazine-1-sulfonamide (TFA Salt) | 29 | 93.2 | 348.1 |
| 132 | 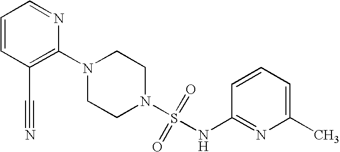 | 4-(3-cyanopyridin-2-yl)-N-(6- methylpyridin-2- yl)piperazine-1-sulfonamide (TFA Salt) | 46 | 74.8 | 359.0 |
| 133 | 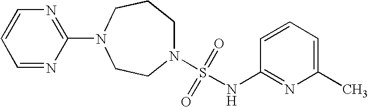 | N-(6-methylpyridin-2-yl)-4- pyrimidin-2-yl-14-diazepane- 1-sulfonamide (TFA Salt) | 330 | 71 | 349.1 |
| 134 | 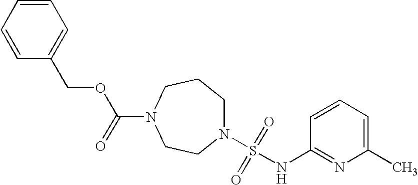 | benzyl 4-{[(6-methylpyridin- 2-yl)amino]sulfonyl}-14- diazepane-1-carboxylate | 110 | 76.7 | 405.1 |
| 135 | 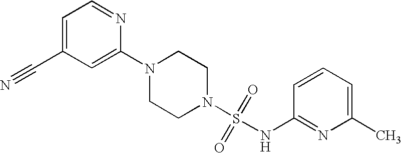 | 4-(4-cyanopyridin-2-yl)-N-(6- methylpyridin-2- yl)piperazine-1-sulfonamide (TFA Salt) | | 29.9 | 359.0 |
| 136 | 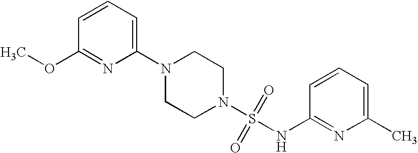 | 4-(6-methoxypyridin-2-yl)-N- (6-methylpyridin-2- yl)piperazine-1-sulfonamide (TFA Salt) | 6.5 | 93.7 | 364.0 |
| 137 | 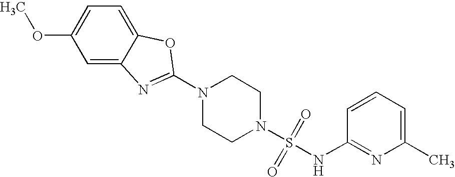 | 4-(5-methoxy-13-benzoxazol- 2-yl)-N-(6-methylpyridin-2- yl)piperazine-1-sulfonamide (TFA Salt) | 9.9 | 93.8 | 404.0 |
| 138 | 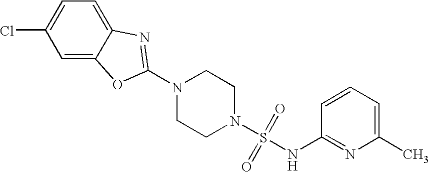 | 4-(6-chloro-13-benzoxazol-2- yl)-N-(6-methylpyridin-2- yl)piperazine-1-sulfonamide (TFA Salt) | 8.4 | 100 | 407.9 |
| 139 | 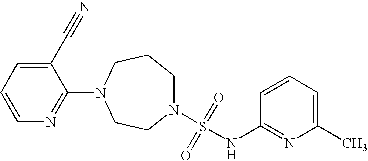 | 4-(3-cyanopyridin-2-yl)-N-(6- methylpyridin-2- yl)-14-diazepane-1- sulfonamide (TFA Salt) | 88 | 86.5 | 373.0 |
| 140 | 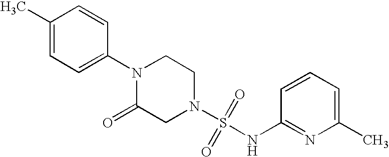 | 4-(4-methylphenyl)-N-(6- methylpyridin-2-yl)-3- oxopiperazine-1-sulfoflamide (TFA Salt) | 250 | 85.7 | 361.0 |
| 141 | 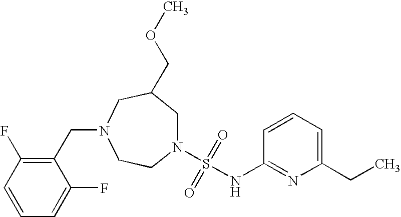 | 4-(26-difluorobenzyl)-N-(6- ethylpyridin-2-yl)-6- (methoxymethyl)-14- diazepane-1-sulfonamide (TFA Salt) | | 13.8 | 455.0 |
| 142 | 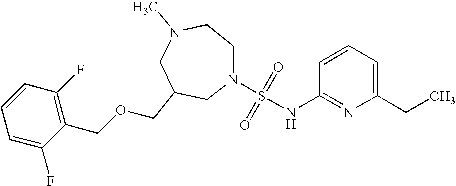 | 6-{[(26-difluoro- benzyl)oxy]methyl)-N-(6- ethylpyridin-2-yl)-4-methyl- 14-diazepane-1-sulfonamide (TFA Salt) | | 5.9 | 455.0 |
| 143 | 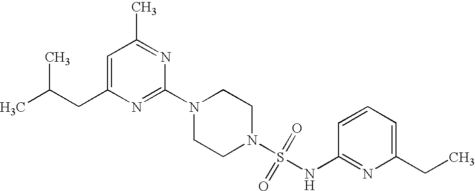 | N-(6-ethylpyridin-2-yl)-4-(4- isobutyl-6-methylpyrimidin-2- yl)piperazine-1-sulfonamide (TFA Salt) | | 36.7 | 419.0 |
| 144 | 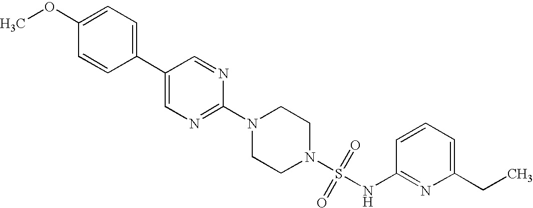 | N-(6-ethylpyridin-2-yl)-4-[5- (4-methoxyphenyl)pyrimidin- 2-yl]piperazine-1-sulfonamide (TFA Salt) | 1 | 100 | 455.0 |
| 145 | 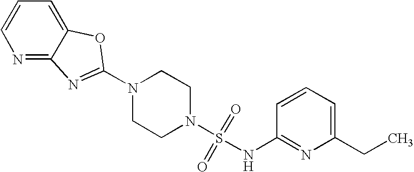 | N-(6-ethylpyridin-2-yl)-4- [13]oxazolo[45-b]pyridin-2- ylpiperazine-1-sulfanamide (TFA Salt) | 62 | 79.2 | 389.0 |
| 146 | 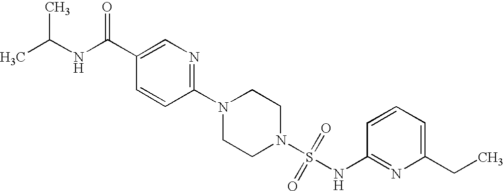 | 6-(4-{[(6-ethylpyridin-2- yl)amino]sulfonyl)piperazin- 1-yl)-N-isopropylnicotinamide (TFA Salt) | | 30.4 | 433.1 |
| 147 | 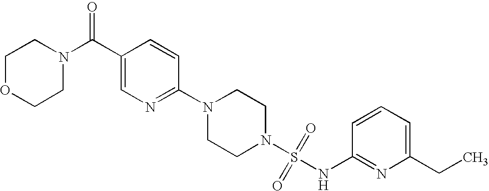 | N-(6-ethylpyridin-2-yl)-4-[5- (morpholin-4- ylcarbonyl)pyridin-2- yl]piperazine-1-sulfonamide (TFA Salt) | | 27.2 | 461.0 |
| 148 | 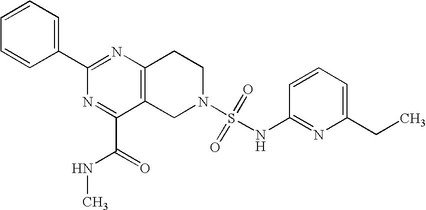 | 6-{[(6-ethylpyridin-2- yl)amino]sulfonyl}-N-methyl- 2-phenyl-5678-tetrahydro- pyrido[43-d]pyrimidine-4- carboxamide (TFA Salt) | 26 | 83.9 | 453.1 |
| 149 | 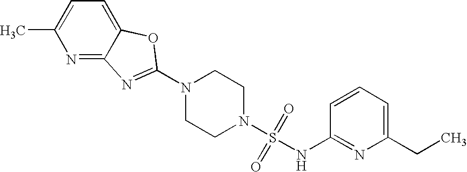 | N-(6-ethylpyridin-2-yl)-4- (5-methyl[13]oxazolo[45- b]pyridin-2-yl)piperazine-1- sulfonamide (TFA Salt) | 280 | 78.2 | 403.0 |
| 150 | 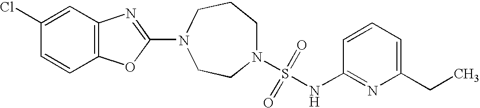 | 4-(5-chloro-13-benzoxazol-2- yl)-N-(6-ethylpyridin-2-yl)- 14-diazepane-1-sulfonamide (TFA Salt) | 23 | 90.3 | 436.0 |
| 151 | 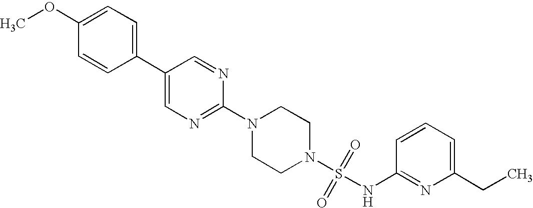 | 4-[5-(4-methoxyphenyl)-pyrimidin-2-yl]-N-(6- methylpyridin-2- yl)piperazine-1-sulfonamide (TFA Salt) | 1 | 100 | 441.1 |
| 152 | 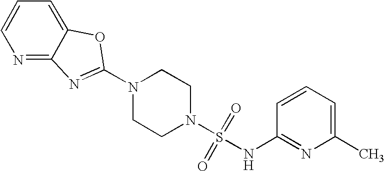 | N-(6-methylpyridin-2-yl)-4- [13]oxazolo[45-b]pyridin-2- ylpiperazine-1-sulfonamide (TFA Salt) | | 47.6 | 375.1 |
| 153 | 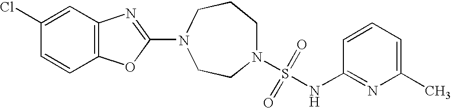 | 4-(5-chloro-13-benzoxazol-2- yl)-N-(6-methylpyridin-2-yl)- 14-diazepane-1-sulfonamide (TFA Salt) | 8.2 | 100 | 422.0 |
| 154 | 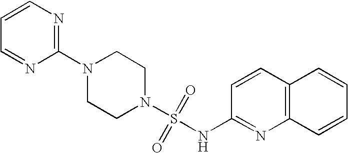 | 4-pyrimidin-2-yl-N-quinolin- 2-ylpiperazine-1-sulfonamide (TFA Salt) | 5.1 | 100 | 371.0 |
| 155 | 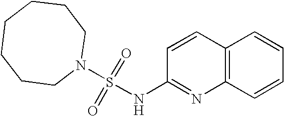 | N-quinolin-2-ylazocane-1- sulfonamide | 7.1 | 90.6 | 320.0 |
| 156 | 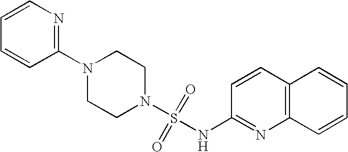 | 4-pyridin-2-yl-N-quinolin-2- ylpiperazine-1-sulfonamide (TFA Salt) | 17 | 92.9 | 370.0 |
| 157 | 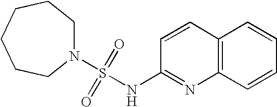 | N-quinolin-2-ylazepane-1- sulfonamide | 32 | 100 | 306.1 |
| 158 | 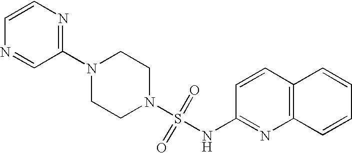 | 4-pyrazin-2-yl-N-quinolin-2- ylpiperazine-1-sulfonamide (TFA Salt) | | 44.8 | 371.0 |
| 159 | 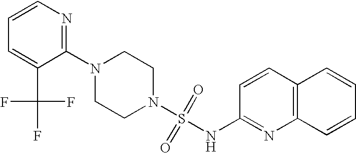 | N-quinolin-2-yl-4-[3- (trifluoromethyl)pyridin-2- yl]piperazine-1-sulfonamide (TFA Salt) | | 53.4 | 437.9 |
| 160 | 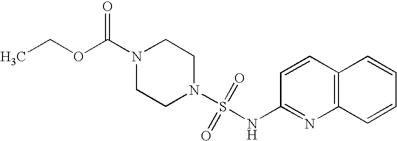 | ethyl 4-[(quinolin-2- ylamino)sulfonyl]piperazine- 1-carboxylate (TFA Salt) | 11 | 85.7 | 365.0 |
| 161 | 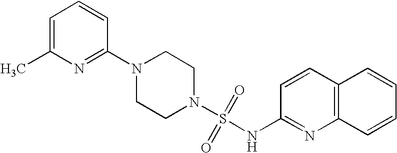 | 4-(6-methylpyridin-2-yl)-N- quinolin-2-ylpiperazine-1- sulfonamide (TFA Salt) | 16 | 91.9 | 384.1 |
| 162 | 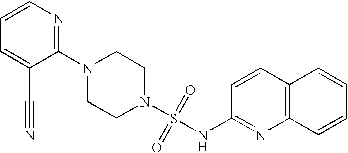 | 4-(3-cyanopyridin-2-yl)-N- quinolin-2-ylpiperazine-1- sulfonamide (TFA Salt) | 26 | 92.2 | 395.0 |
| 163 | 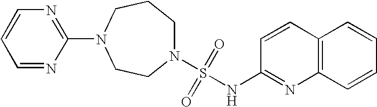 | 4-pyrimidin-2-yl-N-quinolin- 2-yl-14-diazepane-1- sulfonamide (TFA Salt) | | 10.5 | 385.1 |
| 164 | 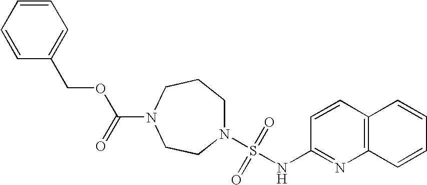 | benzyl 4-[(quinolin-2- ylamino)sulfonyl]-14- diazepane-1-carboxylate | 38 | 81.4 | 441.1 |
| 165 | 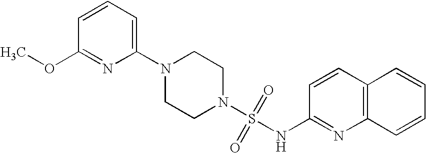 | 4-(6-methoxypyridin-2-yl)-N- quinolin-2-ylpiperazine-1- sulfonamide (TFA Salt) | 1.4 | 100 | 400.1 |
| 166 | 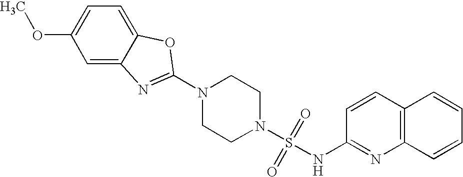 | 4-(5-methoxy-13-benzoxazol- 2-yl)-N-quinolin-2- ylpiperazine-1-sulfonamide (TFA Salt) | 16 | 100 | 440.0 |
| 167 | 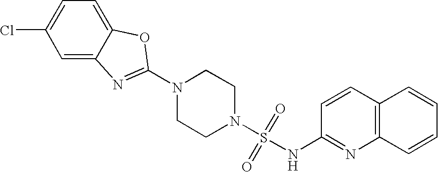 | 4-(6-chloro-13-benzoxazol-2- yl)-N-quinolin-2-ylpiperazine- 1-sulfonamide (TFA Salt) | 3.4 | 100 | 444.1 |
| 168 | 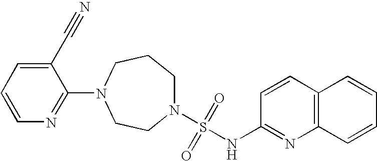 | 4-(3-cyanopyridin-2-yl)-N- quinolin-2-yl-14-diazepane-1- sulfonamide (TFA Salt) | | 56.7 | 409.1 |
| 169 | 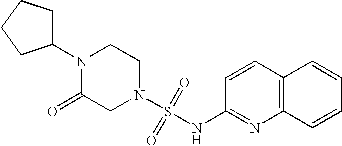 | 4-cyclopentyl-3-oxo-N- quinolin-2-ylpiperazine-1- sulfonamide | 68 | 84.6 | 375.1 |
| 170 | 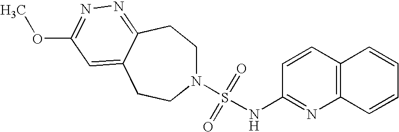 | 3-methoxy-N-quinolin-2-yl- 5689-tetrahydro-7H- pyridazino[34-d]azepine-7- sulfonamide (TFA Salt) | | 21.2 | 386.0 |
| 171 | 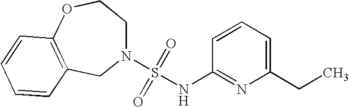 | N-(6-ethylpyridin-2-yl)-23- dihydro-14-benzoxazepine- 4(5H)-sulfonamide (TFA Salt) | | 394 | 334.1 |
| 172 | 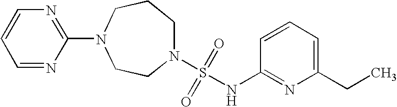 | N-(6-ethylpyridin-2-yl)-4- pyrimidin-2-yl-14-diazepane- 1-sulfonamide (TFA Salt) | | 60.2 | 363.1 |
| 173 | 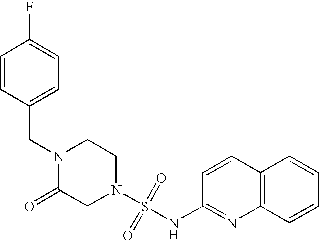 | 4-(4-fluorobenzyl)-3-oxo-N- quinolin-2-ylpiperazine-1- sulfonamide | 130 | 75.5 | 415.1 |
| 174 | 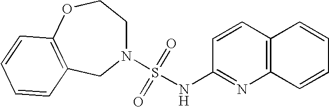 | N-quinolin-2-yl-23-dihydro- 14-benzoxazepine-4(5H)- sulfonamide | | 57.9 | 356.0 |
| 175 | 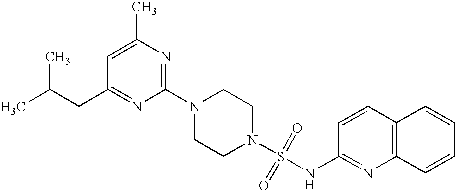 | 4-(4-isobutyl-6-methyl- pyrimidin-2-yl)-N-quinolin-2- ylpiperazine-1-sulfonamide (TFA Salt) | | 50 | 441.2 |
| 176 | 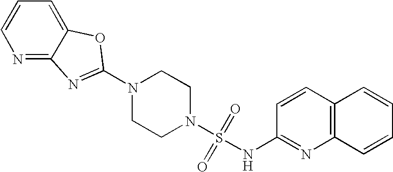 | 4-[13]oxazolo[45-b]pyridin-2- yl-N-quinolin-2-ylpiperazine- 1-sulfonamide (TFA Salt) | 110 | 70.4 | 411.1 |
| 177 | 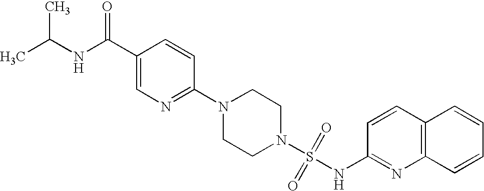 | N-isopropyl-6-{4-[(quinolin- 2-ylamino)sulfonyl]piperazin- 1-yl}nicotinamide (TFA Salt) | | 59.4 | 455.2 |
| 178 | 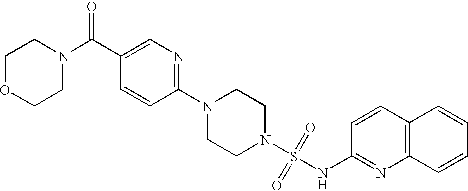 | 4-[5-(morpholin-4- ylcarbonyl)pyridin-2-yl]-N- quinolin-2-ylpiperazine-1- sulfonamide (TFA Salt) | 190 | 73.3 | 483.1 |
| 179 | 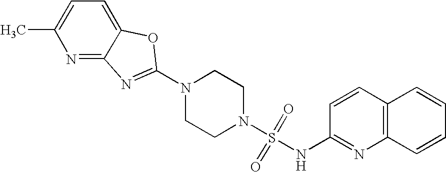 | 4-(5-methyl[13]oxazolo[45- b]pyridin-2-yl)-N-quinolin-2- ylpiperazine-1-sulfonamide (TFA Salt) | | 0 | 425.0 |
| 180 | 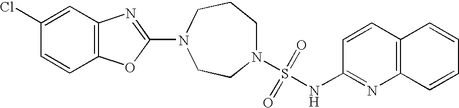 | 4-(5-chloro-13-benzoxazol-2- yl)-N-quinolin-2-yl-14- diazepane-1-sulfonamide (TFA Salt) | 20 | 100 | 458.0 |
| 181 | 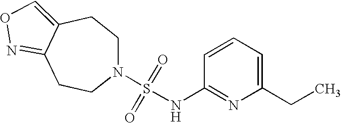 | N-(6-ethylpyridin-2-yl)-4578- tetrahydro-6H-isoxazolo[34- d]azepine-6-sulfonamide | 96 | 83.4 | 323.0 |
| 182 | 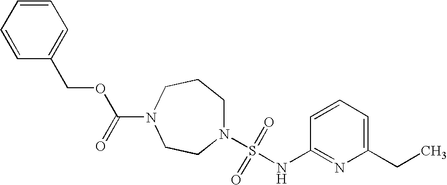 | benzyl 4-{[(6-ethylpyridin-2- yl)amino]sulfonyl}-14- diazepane-1-carboxylate | | 61 | 419.2 |
| 183 | 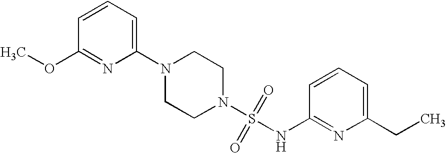 | N-(6-ethylpyridin-2-yl)-4-(6- methoxypyridin-2- yl)piperazine-1-sulfonamide (TFA Salt) | 11.5 | 100 | 378.3 |
| 184 | 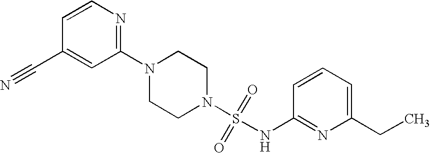 | 4-(4-cyanopyridin-2-yl)-N-(6- ethylpyridin-2-yl)piperazine- 1-sulfonamide (TFA Salt) | | 16.8 | 373.1 |
| 185 | 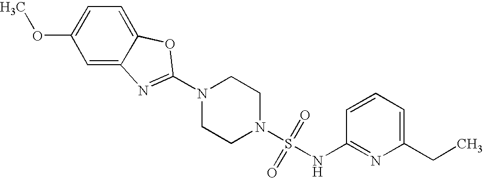 | N-(6-ethylpyridin-2-yl)-4-(5- methoxy-13-benzoxazol-2- yl)piperazine-1-sulfonamide (TFA Salt) | | 48.9 | 418.1 |
| 186 | 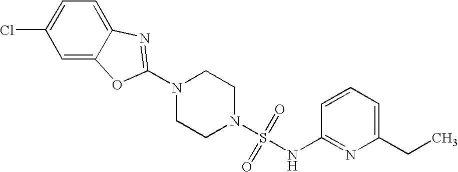 | 4-(6-chloro-13-benzoxazol-2- yl)-N-(6-ethylpyridin-2- yl)piperazine-1-sulfonamide (TFA Salt) | 8.9 | 100 | 422.1 |
| 187 | 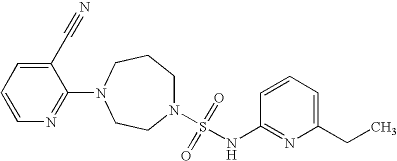 | 4-(3-cyanopyridin-2-yl)-N-(6- ethylpyridin-2-yl)-14- diazepane-1-sulfonamide (TFA Salt) | 160 | 68.8 | 387.1 |
| 188 | 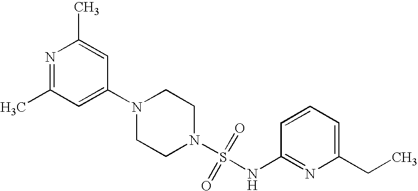 | 4-(26-dimethylpyridin-4-yl)- N-(6-ethylpyridin-2- yl)piperazine-1-sulfonamide (TFA Salt) | | 5.5 | 376.1 |
| 189 | 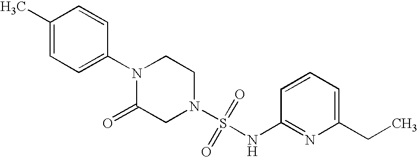 | N-(6-ethylpyridin-2-yl)-4-(4- methylphenyl)-3- oxopiperazine-1-sulfonamide (TFA Salt) | | 24.9 | 375.1 |
| 190 | 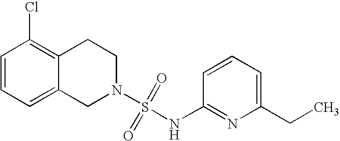 | 5-chloro-N-(6-ethylpyridin-2- yl)-34-dihydroisoquinoline- 2(1H)-sulfonamide (TFA Salt) | 1 | 100 | 352.1 |
| 191 | 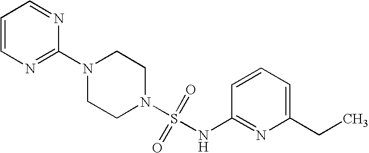 | N-(6-ethylpyridin-2-yl)-4- pyrimidin-2-ylpiperazine-1- sulfonamide (TFA Salt) | 120 | 90.5 | 349.1 |
| 192 | 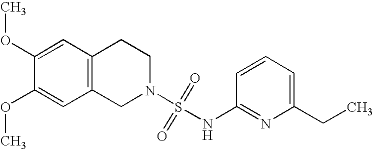 | N-(6-ethylpyridin-2-yl)-67- dimethoxy-34- dihydroisoquinoline-2(1H)- sulfonamide (TFA Salt) | | 29.4 | 378.1 |
| 193 | 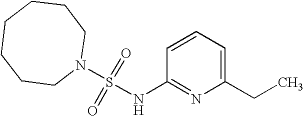 | N-(6-ethylpyridin-2- yl)azocane-1-sulfonamide (TFA Salt) | 7.7 | 92.5 | 298.1 |
| 194 | 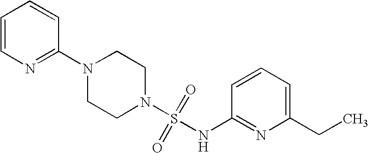 | N-(6-ethylpyridin-2-yl)-4- pyridin-2-ylpiperazine-1- sulfonamide (TFA Salt) | 37 | 81.4 | 348.1 |
| 195 | 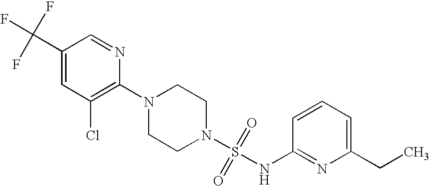 | 4-[3-chloro-5- (trifluoromethyl)pyridin-2- yl]-N-(6-ethylpyridin-2- yl)piperazine-1-sulfonamide (TFA Salt) | 29 | 92.3 | 450.1 |
| 196 | 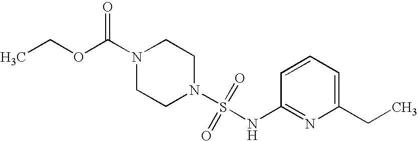 | ethyl 4-{[(6-ethylpyridin-2- yl)amino]sulfonyl}piperazine- 1-carboxylate (TFA Salt) | 20 | 85.8 | 343.1 |
| 197 | 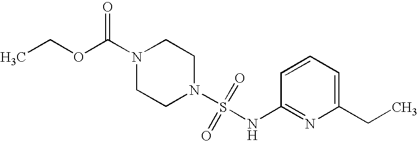 | N-(6-ethylpyridin-2- yl)azepane-1-sulfonamide (TFA Salt) | 35 | 79.5 | 284.3 |
| 198 | 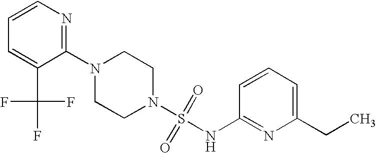 | N-(6-ethylpyridin-2-yl)-4-[3- (trifluoromethyl)pyridin-2- yl]piperazine-1-sulfonamide (TFA Salt) | 71 | 69.1 | 416.0 |
| 199 | 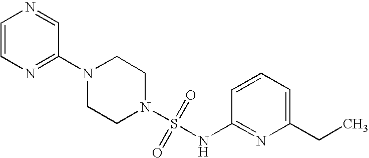 | N-(6-ethylpyridin-2-yl)-4- pyrazin-2-ylpiperazine-1- sulfonamide (TFA Salt) | | 31.3 | 349.1 |
| 200 | 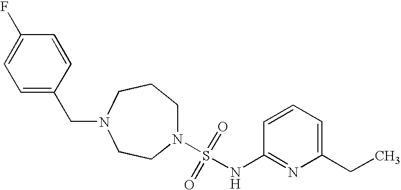 | N-(6-ethylpyridin-2-yl)-4-(4- fluorobenzyl)-14-diazepane-1- sulfonamide (TFA Salt) | 20 | 96.8 | 393.2 |
| 201 | 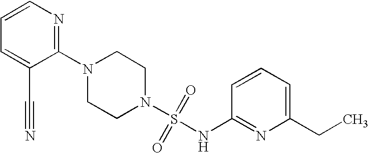 | 4-(3-cyanopyridin-2-yl)-N-(6- ethylpyridin-2-yl)piperazine- 1-sulfonamide (TFA Salt) | 33 | 83.9 | 373.1 |
| 202 | 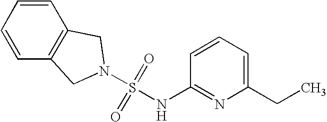 | N-(6-ethylpyridin-2-yl)-13- dihydro-2H-isoindole-2- sulfonamide (TFA Salt) | | 2.4 | 304.1 |

While the invention has been illustrated by reference to specific embodiments, those skilled in the art will recognize that variations and modifications may be made through routine experimentation and practice of the invention. Thus, the invention is intended not to be limited by the foregoing description, but to be defined by the appended claims and their equivalents. The foregoing detailed description and examples have been given for clarity of understanding only.

We claim:

1. A compound of formula (I): 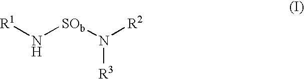
wherein:
R¹ is 2-pyridinyl which is fused or substituted with 1-3 R^{6′} groups, with at least one R⁶ group being at the 6′ position of the pyridinyl;
b is 2;
R² and R³ are taken together with the nitrogen atom to which they are attached to form a (4 to 11)-membered heterocyclyl, and the (4 to 11)-membered heterocyclyl may optionally be substituted by 1 to 3 R⁶ groups;
the carbon atoms of R¹, R², and R³ may each be optionally substituted by 1 to 3 R⁶ groups;
each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C=O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)ᵥ(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl,
-k is selected from 1 and 2;
j is selected from the group consisting of 0, 1, and 2;
t, u, p, q, and v are each independently selected from the group consisting of 0, 1, 2, 3, 4, and 5;
any 1 or 2 carbon atoms of any foregoing (4 to 11)-membered heterocyclyl groups may be optionally substituted with an oxo (═O);
any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₈)alkenyl, (C₂-C₆)alkynyl, —(CR²¹R²²)ᵤ(C₆-C₁₂)aryl, and —(CR²¹R²²)ᵤ(4 to 11)-membered heterocyclyl;
each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)ₚ(4 to 11)-membered heterocyclyl;
any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);
each R²³ and R²⁴ is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;
with the proviso that —NR²R³ is not an unsubstituted group selected from 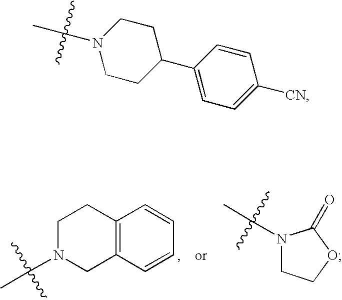
and the further proviso that when —R¹ is 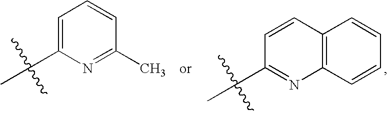
then —NR²R³ is not an unsubstituted or substituted, fused or unfused group selected from 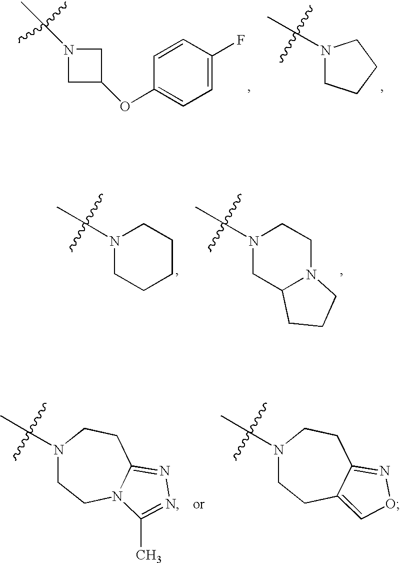
or a pharmaceutically acceptable salt or solvate thereof.

2. The compound according to claim 1, wherein R¹ is pyridinyl substituted with 1 to 3 R⁶ groups.

3. The compound according to claim 2, wherein R¹ is quinolinyl.

4. The compound according to claim 1, wherein R² and R³ are taken together to form a 6-membered heterocyclyl containing at least one nitrogen atom.

5. The compound according to claim 4 wherein the 6-membered heterocyclyl is piperazinyl.

6. The compound according to claim 1, wherein R² and R³ are taken together to form a 10-membered heterocyclyl containing at least one nitrogen atom.

7. The compound according to claim 1, wherein R² and R³ are taken together to form an 11-membered heterocyclyl containing at least one nitrogen atom.

8. The compound according to claim 7, wherein the 11-membered heterocyclyl is benzazepinyl.

9. A compound selected from the group consisting of: 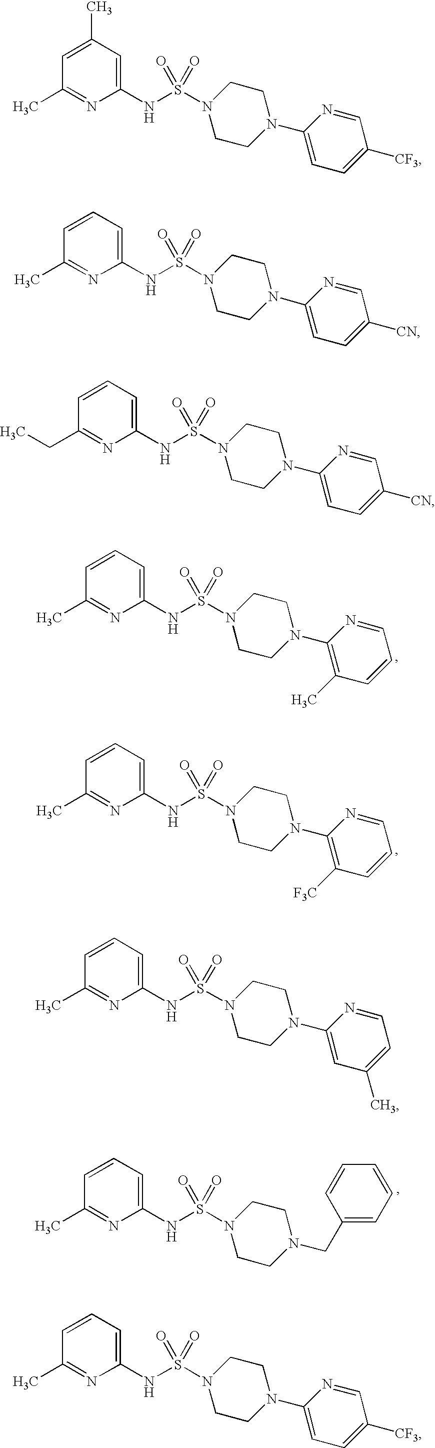 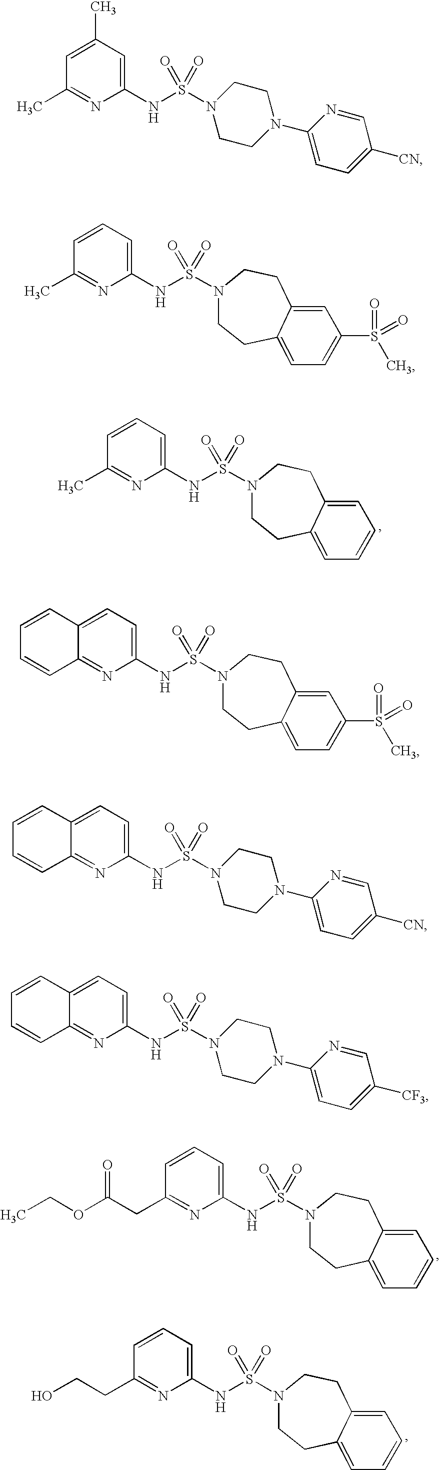 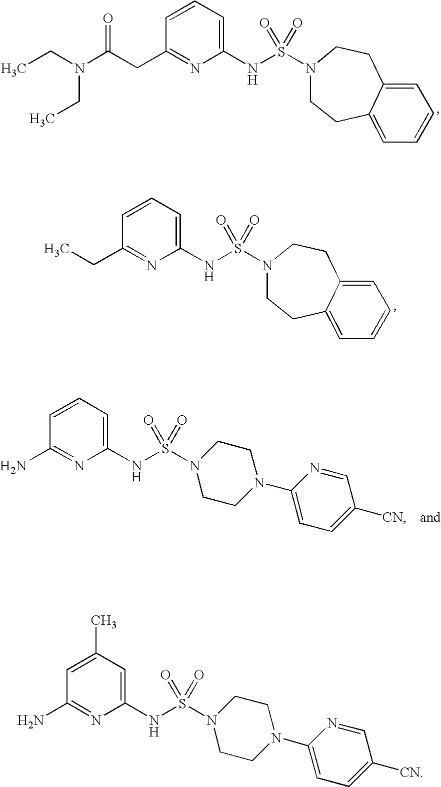
or a pharmaceutically acceptable salt or solvate thereof.

10. A compound selected from the group consisting of 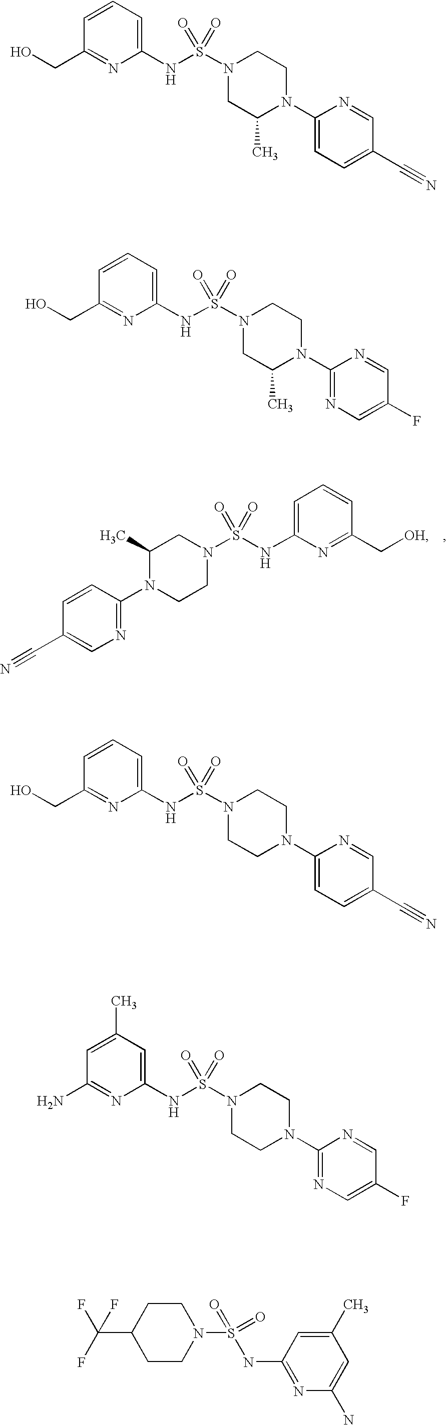 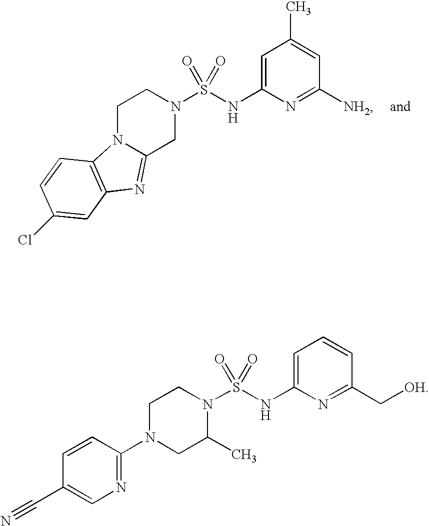
or a pharmaceutically acceptable salt or solvate thereof.

11. A pharmaceutical composition comprising an effective amount of a compound according to claim 1, or a pharmaceutically acceptable salt or solvate thereof, and a pharmaceutically acceptable carrier.

12. A pharmaceutical composition comprising an effective amount of a compound according to claim 10, or a pharmaceutically acceptable salt or solvate thereof, and a pharmaceutically acceptable carrier.

13. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to claim 1, or a pharmaceutically acceptable salt or solvate thereof.

14. A compound of formula (III): 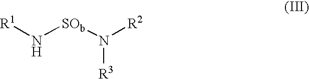
wherein:
R¹ is pyridinyl which is fused or unfused, unsubstituted or substituted with 1-3 R⁶ groups; —(CR⁴R⁵)ₜ(C₆-C₁₂)aryl, and —(CR⁴R⁵)ₜ(4 to 10)-membered heterocyclyl;
b is 2;
R² and R³ are taken together with the nitrogen atom to which they are attached to form a (12-14)-membered heterocyclyl, and the (12-15)-membered heterocyclyl may optionally be substituted by 1 to 3 R⁶ groups;
each R⁴ and R⁵ is independently selected from H and (C₁-C₆)alkyl; the carbon atoms of R¹, R², R³, R⁴, and R⁵ may each be optionally substituted by 1 to 3 R⁶ groups;
each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²¹)ᵥ(C₆-C₁₂)aryl, —(CR¹⁷R¹⁸)_{q}S(O)ⱼ(CR¹⁹R²⁰)ᵥ(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl;
k is selected from 1 and 2;
j is selected from the group consisting of 0, 1, and 2;
t, u, p, q, and v are each independently selected from the group consisting of 0, 1, 2, 3, 4, and 5;
any 1 or 2 carbon atoms of any foregoing (4 to 11)-membered heterocyclyl group may be optionally substituted with an oxo (═O);
any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₁-C₆)alkyl, (C₂-C₆)alkenyl, (C₂-C₆)alkynyl, —(CR²¹R²²)ᵤ(C₆-C₁₂)aryl, and —(CR²¹R²²)ᵥ(4 to 11)-membered heterocyclyl;
each R⁷, R⁸, R⁹, R¹⁰, R¹¹, R¹², R¹³, R¹⁴, R¹⁵, R¹⁶ , R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴)ₚ(C₆-C₁₂)aryl, and —(CR²³R²⁴)ₚ(4 to 11)-membered heterocyclyl;
any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷ , R¹⁸, R¹⁹, R²⁰, R²¹, and R²² group may be optionally substituted with an oxo (═O);
each R²³ and R²⁴ is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;
or a pharmaceutically acceptable salt or solvate thereof.

15. The compound according to claim 14 wherein —NR²R³ is a 13-membered heterocyclic optionally substituted with 1 to 3 R⁶ groups.

16. A method of preparing a compound of formula (I) 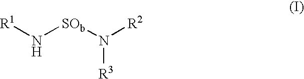
wherein:
R¹ is a —(CR⁴R⁵)ₜ(4 to 10)-membered heterocyclyl;
b and k are each independently selected from 1 and 2;
j is selected from the group consisting of 0, 1, and 2;
t, u, p, q, and v are each independently selected from the group consisting of 0, 1, 2, 3, 4, and 5;
each R² and R³ is independently selected from the group consisting of H, (C₁-C₆) alkyl, (C₂-C₆) alkenyl, (C₂-C₆) alkynyl, —(CR⁴R⁵)ₜ(C₃-C₁₀)cycloalkyl, —(CR⁴R⁵)₂(C₆-C₁₀)aryl, and —(CR⁴R⁵)ₜ(4 to 11)-membered heterocyclyl;
or R² and R³ may optionally be taken together with the nitrogen atom to which they are attached to form a (4 to 11)-membered heterocyclyl, and the (4 to 11)-membered heterocyclyl may be optionally substituted by 1 to 3 R⁶ groups;
each R⁴ and R⁵ is independently selected from H and (C₁-C₆)alkyl; the carbon atoms of R¹, R², R³, R⁴, and R⁵ may each be optionally substituted by 1 to 3 R⁶ groups
each R⁶ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(CR⁷R⁸)ᵥ(C₆-C₁₂ aryl), —(CR⁷R⁸)ᵥ(4 to 11)-membered heterocyclyl, —(C═O)—R⁹, —(C═O)—O—R⁹, —O—(C═O)—R⁹, —R⁹—(C═O)—O—R¹⁰, —(CR⁹R¹⁰)_{q}(C═O)(CR¹¹R¹²)ᵥ(C₆-C₁₂)aryl, —(CR⁹R¹⁰)_{q}(C═O)(CR¹¹R¹²)ᵥ(4 to 11)-membered heterocyclyl, —O—(C═O)—NR¹³R¹⁴, —NR¹³(C═O)—R¹⁴, —(C═O)—NR¹³R¹⁴, —R¹³—(C═O)—NR¹⁴R¹⁵, —NR¹³R¹⁴, —NR¹³OR¹⁴, —S(O)ₖNR¹³R¹⁴, —S(O)ⱼ(C₁-C₆)alkyl, —O—SO₂—R¹⁵, —NR¹⁵—S(O)ₖ—R¹⁶, —(CR¹⁷R¹⁸)_{q}S(O)ⱼ(CR¹⁹R²⁰)ᵥ(C₆-C₁₂)aryl, —(CR¹⁷R¹⁸)_{q}S(O)ⱼ(CR¹⁹R²⁰)(4 to 11)-membered heterocyclyl, —(CR¹⁷R¹⁸)ᵥO(CR¹⁹R²⁰)_{q}(C₆-C₁₂)aryl, and —(CR¹⁷R¹⁸)ᵥO(CR¹⁹R²⁰)_{q}(4 to 11)-membered heterocyclyl, any 1 or 2 carbon atoms of any foregoing (4 to 11)-membered heterocyclyl group may be optionally substituted with an oxo (═O);
any (C₁-C₆)alkyl, any (C₆-C₁₂)aryl, and any (4 to 11)-membered heterocyclyl of the foregoing R⁶ groups may be optionally substituted with 1 to 3 substituents independently selected from the group consisting of halo, cyano, nitro, —CF₃, —CFH₂, —CF₂H, trifluoromethoxy, azido, —OR²¹, —(C═O)—R²¹, —(C═O)—O—R²¹, —O—(C═O)—R²¹, —NR²¹(C═O)—R²², —(C═O)—NR²¹R²², —NR²¹R²², —NR²¹OR²², (C₂-C₆)alkyl, (C₂-C₆)alkenyl, (C₂-C₆)alkynyl, —(CR²¹R²²)ᵤ(C₆-C₁₂)aryl, and —(CR²¹R²²)ᵤ(4 to 11)-membered heterocyclyl;
each R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group is independently selected from the group consisting of H, (C₁-C₆)alkyl, —(C═O)N(C₁-C₆)alkyl, —(CR²³R²⁴ )ₚ(C₆-C₁₂)aryl, and —(CR²³R²⁴)ₚ(4 to 11)-membered heterocyclyl;
any 1 or 2 carbon atoms of the (4 to 11)-membered heterocyclyl of each said R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, and R²² group may be optionally substituted with an oxo (═O);
each R²³ and R²⁴ is independently selected from H and (C₁-C₆)alkyl; comprising steps of:
(a) treating a compound of formula (II) 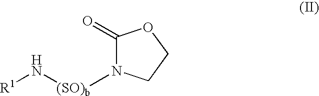
wherein:
R¹ and b are defined as above;
with R²R³NH in the presence of a base in a solvent, wherein each R² and R³ is defined as above.
